SEQUENCE LISTING (152 sequences)

SEQ ID NO: 1 - protein, 296 aa, Artificial Sequence
  Synthetic polypeptide, QYD01, D1
MAHAALSPLSQRFERIAVQPLTGVLGAEITGVDLREPLDDSTWNEILDAFHTYQVIYFPGQAITNEQHIAFSRRFGPVDPVPLLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSVKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 2 - DNA, 888 bp, Artificial Sequence
  Synthetic polynucleotide
atggcccatgccgcgttgagcccgttgtcacagcggttcgagaggatagcagtgcagccacttacaggagtgctgggcgccgaaattaccggtgtggacctgcgcgaaccattggatgatagcacatggaacgaaattctggatgcattccatacgtaccaagtgatttactttccggggcaagcaattacaaatgagcagcacatagctttctcgcggcggttcggtccagtcgaccccgttccgcttcttaagagcatagagggatacccggaagttcagatgattcgccgcgaggcaaacgaaagcgggcgggttataggtgatgactggcatacggattctactttcttggatgctccaccggcggctgtcgttatgagggcaatagacgtgccggagcacggaggagacacaggcttcttgtctatgtacacggcctgggaaaccctgtctcccacaatgcaagctactatcgaaggacttaatgtggtgcattctgccacccgggttttcggctcactgtaccaagctcaaaatcggagattctctaatacaagcgtgaaagtcatggacgtggacgcgggagaccgcgaaaccgttcacccgctcgtcgttacgcaccctggcagcggaagaaagggcttgtacgtcaatcaggtctactgccaaaggattgagggaatgaccgacgcggagtctaaacccttgttgcagtttctctacgaacatgccacgcgctttgatttcacttgtagagtgcggtggaaaaaggaccaggttcttgtttgggataatctttgcaccatgcaccgcgcggttcctgactatgctggtaaatttaggtatttgactcggactacagttgggggagttagacctgctaga

SEQ ID NO: 3 - protein, 289 aa, Artificial Sequence
  Synthetic polypeptide, QYD02, D2
MTIAIRQLQTHFVGQVSGLDLRKPLTPGEAREVESAMDKYAVLVFHDQDITDEQQMAFALNFGQREDARGGTVTKEKDYRLQSGLNDVSNLGKDGKPLAKDSRTHLFNLGNCLWHSDSSFRPIPAKFSLLSARVVNPTGGNTEFADMRAAYDALDDETKAEIEDLVCEHSLMYSRGSLGFTEYTDEEKQMFKPVLQRLVRTHPVHRRKSLYLSSHAGKIASMSVPEGRLLLRDLNEHATQPEFVYVHKWKLHDLVMWDNRQTMHRVRRYDQSQPRDMRRATVAGTEPTV

SEQ ID NO: 4 - DNA, 867 bp, Artificial Sequence
  Synthetic polynucleotide
atgaccatagctattcgccagcttcaaactcattttgtgggacaggtctcgggcctcgacttgcgcaaaccacttacaccaggcgaggctcgggaagtggaatccgctatggataagtatgcggtcttggtcttccacgaccaagatattacagacgagcagcaaatggcctttgccttgaatttcggtcagagagaggatgctagagggggaaccgtcacgaaagagaaagactacaggcttcaaagcgggcttaacgatgtttcaaatctcggaaaagatggaaagcctcttgctaaggattctagaacgcatttgtttaatcttggtaattgtctctggcacagcgattccagcttccgccccataccggccaaattctcgctcctcagcgcaagagtggtcaaccctacaggtggaaatacagagtttgcagatatgagagctgcttacgatgcgctggatgacgaaactaaggcagagatagaggacctcgtttgtgagcattccctcatgtactctcggggtagcttggggttcacggagtacactgacgaggaaaaacaaatgttcaagcccgttctccagcggctggtcaggacacatccagtccaccgccgcaaatctttgtatttgtcctcgcacgcagggaaaatcgcctccatgagcgtgcccgagggccgcttgcttctgagggacctcaatgaacacgcaacgcaaccggaattcgtttatgtccacaaatggaaacttcatgaccttgttatgtgggacaatcgccagacaatgcatagagtccgcagatacgatcaatctcagccaagagatatgcgccgcgccacagtcgcagggacagagcctacagtc

SEQ ID NO: 5 - protein, 287 aa, Artificial Sequence
  Synthetic polypeptide, QYD03, D3
MSPAFDIAPLDATFGAVVTGVKLADLDDAGWLDLQAAWLEYALLVFPDQHLTREQQIAFARRFGPLEFEMAAISNVRPDGSLRVESDNDDMMKILKGNMGWHADSTYMPVQAKGAVFSAEVVPSVGGQTGFADMRAAYDALDEDLKARVETLQARHSLHYSQSKLGHQTKAADGEYSGYGLHDGPVPLRPLVKIHPETGRKSLLIGRHAHAIPGLEPAESERLLQQLIDFACQPPRIYHHDWAPGDAVLWDNRCLLHQATPWDMTQKRIMWHSRIAGDPASETALAH

SEQ ID NO: 6 - DNA, 861 bp, Artificial Sequence
  Synthetic polynucleotide
atgtcaccggccttcgacatagcccccttggatgctacattcggggctgtggttaccggagtcaagcttgctgatttggatgatgcggggtggctcgacttgcaggcggcttggttggagtacgcattgctcgtttttcccgaccagcacctgaccagagagcaacagatagcgttcgcgcggaggtttggcccactcgagtttgagatggccgccatatccaacgtcaggccagatggctctcttcgggtggagtccgacaacgacgatatgatgaagatactcaaggggaatatgggttggcatgccgactccacctatatgcccgtgcaggctaaaggggcagtgtttagcgcagaggttgtgccctctgtcggaggtcagactggcttcgcggacatgagagcagcttatgacgcccttgatgaggacctcaaagctagagttgagactctgcaggctcggcattcccttcattactctcaaagcaagttgggacaccagactaaggctgccgatggtgaatactcgggttacggtctgcatgacggccccgtgccacttcggcctcttgttaaaatccatccggagaccggccggaaatctctcttgataggtaggcacgcccacgccattcccggactggaaccagccgaatcggaacggcttttgcagcaactgatagatttcgcatgtcagccccctcggatctaccaccatgactgggctccaggagacgcggtgttgtgggataaccgctgcttgctgcatcaagcgacgccctgggatatgacgcagaagaggataatgtggcactccagaatagcaggagacccggcgagcgagaccgcacttgcacac

SEQ ID NO: 7 - protein, 287 aa, Artificial Sequence
  Synthetic polypeptide, QYD04, D4
MSPAFDIAPLDATFGAVVTGVKLADLDDAGWLDLQAAWLEYALLVFPDQHLTREQQIAFARRFGPLEFAMAAISNVRPDGSLRVESDNDDMMKILKGNMGWHADSTYMPVQAKGAVFSAEVVPSVGGQTGFADMRAAYDALDEDLKARVETLQARHSLHYSQSKLGHQTKAADGEYSGYGLHDGPVPLRPLVKIHPETGRKSLLIGRHAHAIPGLEPAESERLLQQLIDFACQPPRIYHHDWAPGDAVLWDNRCLLHQATPWDMTQKRIMWHSRIAGDPASETALAH

SEQ ID NO: 8 - DNA, 861 bp, Artificial Sequence
  Synthetic polynucleotide
atgtcaccggccttcgacatagcccccttggatgctacattcggggctgtggttaccggagtcaagcttgctgatttggatgatgcggggtggctcgacttgcaggcggcttggttggagtacgcattgctcgtttttcccgaccagcacctgaccagagagcaacagatagcgttcgcgcggaggtttggcccactcgagtttgcgatggccgccatatccaacgtcaggccagatggctctcttcgggtggagtccgacaacgacgatatgatgaagatactcaaggggaatatgggttggcatgccgactccacctatatgcccgtgcaggctaaaggggcagtgtttagcgcagaggttgtgccctctgtcggaggtcagactggcttcgcggacatgagagcagcttatgacgcccttgatgaggacctcaaagctagagttgagactctgcaggctcggcattcccttcattactctcaaagcaagttgggacaccagactaaggctgccgatggtgaatactcgggttacggtctgcatgacggccccgtgccacttcggcctcttgttaaaatccatccggagaccggccggaaatctctcttgataggtaggcacgcccacgccattcccggactggaaccagccgaatcggaacggcttttgcagcaactgatagatttcgcatgtcagccccctcggatctaccaccatgactgggctccaggagacgcggtgttgtgggataaccgctgcttgctgcatcaagcgacgccctgggatatgacgcagaagaggataatgtggcactccagaatagcaggagacccggcgagcgagaccgcacttgcacac

SEQ ID NO: 9 - protein, 602 aa, Artificial Sequence
  Synthetic polypeptide, QYD05, D5
MPFAGELPSAIETQVSKILALIAAYRHRSPNVPDRFAQFVPELEKQLADTVSKKEPVRFVLPAFPFKAPVEGNKRKTLGSLPDKAEEIALKTLDGFADSIAEIYEAGATVVLVSDASVYGDLLNVADAEAFTYHQELQKLASSLGLEHLEFVRPGALAGIAPKDAHTLEEYSDHVNKTRNLLDGPLAQDVNPSEDENVQATSKHYDTALPTTDDPEAFKTAMLKRGKAYAKLIASSSETTIRLSIHESNNVGKITLDLFPPSTNPDFITPWHGAVAVLADASVRIVDASTVDMTKFEVITNAEGRPWLLREKSELFNWFGMELDFEPLFPCGMVVRPRNGYGPYKFEDVNMKQVRRLALSTAPLLLRGFTMEIEKEVFRQKARELGEIQMWPFGDILEVRENADFNMNNVLTREAMPFHYDGVFKTIQDEKTGEWISTPPLFQMFRNRAASQQKGGLTLFSSSRNLIPLLGPDSIPLDELRKLKWKTFTVVNEAFGGHELLLPFIASHPETGADTFRFHEPWPESKCISGSSEPTIVQVVGWPAAESDALCAKLTDLLYDRRVAYRHQWTAGDFIFNDNATTHHTRTAFENGHREHWRVHVS

SEQ ID NO: 10 - DNA, 1806 bp, Artificial Sequence
  Synthetic polynucleotide
atgccatttgcgggcgaacttccatcagccatagagacgcaggtctccaagatacttgccctgattgctgcttatagacaccgctcaccaaacgtgccagaccggtttgcgcagttcgtcccggaactcgagaagcaactggctgataccgtctcaaaaaaagaacctgttagattcgtgcttccggctttcccctttaaagcccctgtcgaaggaaacaagagaaaaacccttggttcgttgcccgataaagcagaagaaatagcgcttaagactttggacggtttcgcagattctattgcagaaatatatgaagctggcgcgactgtggttctggtctctgacgccagcgtttatggagacttgttgaacgtcgccgatgcggaagcatttacgtaccaccaggaattgcaaaagctggcttccagcctgggattggagcatttggaatttgttaggcccggcgcactggctgggattgctcctaaagatgcgcacaccctggaggagtactctgatcacgtcaataaaactaggaatttgctcgatggaccgttggcccaggacgtgaatccatctgaagacgaaaacgtccaagctacttcgaagcactatgacaccgctctgccgaccactgacgatcccgaggcctttaaaacggcgatgctgaagcggggtaaagcatacgctaaactcatagcttcctccagcgagacgactatacgcctctcaatacacgaatcgaacaacgtcgggaaaattaccctggatttgtttcctccatccactaatcccgattttataactccatggcacggtgccgttgcagtgctcgcggacgcatccgttagaatcgtggatgcgtctacagtggatatgactaagtttgaagttatcacaaatgccgagggaaggccctggcttcttagggaaaagagcgaattgttcaactggttcgggatggaactcgatttcgagcctttgtttccatgcgggatggtcgtccggcccagaaatggttacggaccctacaagtttgaggatgtcaacatgaaacaggttcggagattggcactttcaacagcaccgctcttgctcaggggattcacgatggagatcgaaaaagaggttttccgccagaaggcccgcgaactgggtgagatccaaatgtggcctttcggagatattttggaggtcagagaaaacgcggacttcaatatgaacaacgtcctgacacgcgaggcgatgccatttcactacgacggagtgttcaagacaatccaagacgagaagacgggtgaatggatctcaacaccgcccctgtttcagatgttcaggaatagagcggcctcccagcagaagggtggactgaccttgttttcttcgtccagaaatttgatccccctgctcggacccgactctatcccattggatgaactccgcaagctgaagtggaagacatttaccgtcgtgaatgaggcttttgggggtcacgaacttctgttgccctttatcgcttcccatccggagaccggggccgatacgttccggtttcatgaaccttggcccgaaagcaagtgcatatcaggatcatctgagccaacgattgtccaggttgtcggctggcccgcagcggaatctgacgcgctttgtgcaaagcttactgatctcttgtatgataggagggtggcctaccgccaccaatggacagcaggggactttatatttaatgataacgctacaacccaccacacgagaacggcttttgaaaacggtcatagagagcattggcgcgtccatgtctca

SEQ ID NO: 11 - protein, 278 aa, Artificial Sequence
  Synthetic polypeptide, QYD06, D6
MNFQVTHLKPFGAIVEPKAQGASVKDLDLKALHQLFLQEQIVVLRGFTTFKNSEEFASYCETWGEISIWPFGKVLELVEQENPQDHIFDHSYVPMHWDGMYRPQVPEYQIFHCVKAPLSGHGGRTTFSNTVLALKNASPELKAFWGKVTGIYQREMEFYKSKTVSPIITKHPKRDFSVIRYNEPPSADKGHFVNPPDLEFAGVPVGELEAFHQGLKSALYAPENFYAHEWQDGDVVITDNFTLLHGREAFTSKSPRHLQRVQVQSSPPFDNPGLESYK

SEQ ID NO: 12 - DNA, 834 bp, Artificial Sequence
  Synthetic polynucleotide
atgaattttcaagttacgcatttgaaacctttcggtgccatcgttgaacccaaggcgcagggcgcctctgtcaaggatcttgacctcaaagcgttgcatcaactgtttctccaagagcaaattgtcgtcctgagggggttcactacatttaaaaactctgaagagttcgcctcctattgtgaaacctggggcgagatttcaatttggccgttcggaaaggtcttggagcttgtggagcaagaaaatccccaggatcacattttcgaccatagctacgtgccgatgcactgggacggtatgtatcggccccaagtcccggaataccaaatatttcattgtgttaaggcacccctgtctggccacgggggcaggacaacctttagcaacactgtgctcgctcttaaaaacgcctcgcctgaacttaaggcgttttgggggaaggtgactggcatataccaacgggaaatggaattttataagtctaaaactgtctctccaatcattacaaagcatcccaagagagatttttccgtcatccgctataatgaaccaccttccgctgacaaaggtcactttgtgaatccgcctgatttggagtttgccggcgtgccagtgggcgagttggaagcattccaccaagggcttaaatccgccctttacgctccggaaaacttttacgctcacgaatggcaagacggcgacgtggtcattaccgacaattttacactgttgcacgggagagaagccttcacatcaaagtcgccgagacaccttcaaagggtccaagttcagtccagccctccattcgataaccctggcttggaatcatacaaa

SEQ ID NO: 13 - protein, 393 aa, Artificial Sequence
  Synthetic polypeptide, QYD07, D7
MTKLYEKQSFKAPAAGFTFESFDLPSTNQRILGPPHPPDTVSPLALRPSTGIETDITLDDVVETVNSLQAQNGTLTAALARHGTLLFRGLPIHNATDFSRFAHAFGYRPHEIIGIVVDRPVLAPNVAPASEAPKDVLIYNHNESPQVPHAPEYIFFYGHRVPARGGESPISSSLELFHRAQLEIPELIQQLADKGILSRVVYKKEPAYVGGSSLRQAFGKEVQEGDDEATQRRKMEAQIARYGRGKFTTWEWTEDGLVVTHRLPAIRTQPRTNLPSLFTGLAAYYKNMTVNNHGGRRNLTQQLYGDGTPIPEEHLAQLVQITDDIRVLHKWEQGDILVFDNVIAQHGREPWEGDQKDRVVMASLWDGESVPGAYGYGEWAQVVQALDPVESSV

SEQ ID NO: 14 - DNA, 1179 bp, Artificial Sequence
  Synthetic polynucleotide
atgactaagctctacgaaaaacaatcattcaaggcccccgctgccggatttacttttgaaagctttgacttgccatcaacaaaccagcgcattcttggaccacctcatcccccagacacagtctcacctttggcattgagaccatcaacaggtatcgagactgatatcaccctcgacgacgtggttgaaaccgtcaatagcctccaagcgcagaatgggactctgactgctgcgttggcgagacacgggactttgctctttaggggtttgcccatccacaatgcaactgactttagccgctttgctcatgcgttcggctatcgcccacacgaaattattggtattgttgtcgaccgcccagtgctggctcctaatgtggcccctgcatctgaagcaccgaaggatgtgcttatatacaatcataacgaatctccacaagtcccccacgccccggagtacatcttcttttatggccatagggttcctgctagaggtggagaaagccctattagctcatcgcttgaattgttccaccgggcacaactcgaaattccagaactcatacaacagctggcggacaaaggaatactctccagagtcgtctacaaaaaagaaccagcctatgtcgggggttcatctttgcgccaagccttcgggaaggaagtccaggaaggggatgatgaggctacccaaagacgcaagatggaggctcagatagcccgctacggtcgcggtaagtttacgacgtgggaatggacagaagatggtctggtggtgacccatagactcccagcaattcgcactcagcctagaacaaacctgccctccctgtttacgggattggccgcatactataagaacatgaccgttaacaaccatggtgggcggaggaaccttacacagcagttgtacggggatggaactccgatacccgaggaacacctcgctcagttggttcaaattactgacgatattcgcgtgttgcataagtgggaacagggggatattctcgtcttcgataacgtcattgcccagcacggtcgggagccctgggagggggatcagaaagatcgcgttgttatggcctcactgtgggatggagagtctgttccaggtgcttatggttacggcgagtgggcgcaagtcgttcaggctttggaccccgttgagtcttcggtc

SEQ ID NO: 15 - protein, 347 aa, Artificial Sequence
  Synthetic polypeptide, QYD08, D8
MAYHQLSYTFLNHQKLPLIIQPQAKAEGNIDVLNQFLKEENALFKEQLLNYGAIILRGFHVNTAEQFLEVIQSSDLGPNYSYDFCPIPRTQIRENIYTSINIHPSFNLALHNEKSYDQDFPSHIFFNCIHAPQTGGDTALADGNKIWFSLPTFLQKKLQSKGILYRRHYYGTGIQYKIIRSIGKNSGCMTWMERFQTDDQNKVEAMLQQMGVQFRWIQGNGLIIEQLLPACRNHPISGKLVWFNQSNHANHYYNGTSDYIKSKINNTFSRFILLHKYFHPYIAFYGDGEPLSKQEADCINSAIQKNTVSTAWQPGDVMIVDNFSCLHGKTPHTGNRLILVGLTKYFD

SEQ ID NO: 16 - DNA, 1041 bp, Artificial Sequence
  Synthetic polynucleotide
atggcatatcaccagctctcctatactttcctgaaccaccaaaagttgcctctcatcatacaaccgcaggcaaaagcggagggtaacatagacgttcttaaccagtttcttaaagaggaaaatgcattgttcaaagagcagttgttgaactacggtgcgatcattctcagaggcttccatgtgaatactgcagagcagttcttggaagtgattcagtcgtctgatttgggccctaactatagctacgatttctgccccataccaagaacacagattcgcgagaacatctacacctccataaatatccacccttcattcaatcttgcccttcacaacgaaaaatcttacgatcaggacttcccatcgcacatattctttaattgtatccatgctccgcaaacgggaggcgatacagcgctggccgacgggaacaaaatatggttctcgctgcccacatttcttcagaaaaagctccaatcgaagggaattctctatagacgccactactacggcacaggaatccagtacaaaataataaggtccattggtaagaactctggttgcatgacctggatggagcggtttcagactgatgaccaaaataaggtggaggccatgctgcagcagatgggcgtgcagtttcggtggatccaggggaacgggctgattatcgagcaattgctccccgcctgtcggaatcacccaatcagcggcaagcttgtctggttcaaccaatccaaccacgcaaatcactactataatggtacttcagactacattaaatcaaaaatcaacaacactttcagccggtttatcctgttgcataaatacttccacccgtatattgctttttatggggacggtgagcccctctccaagcaagaagcagattgtattaactctgctatccagaagaacaccgtttcgactgcttggcagcctggagacgtgatgatcgtggacaatttttcatgcctccatggtaaaaccccacacacaggtaatagactcattctggttggcctcacaaaatacttcgat

SEQ ID NO: 17 - protein, 348 aa, Artificial Sequence
  Synthetic polypeptide, QYD09, D9
MSVHRGPVSGQRRAIALATTDWVRPGGTPPGRELPLVLRPSHDDVDLAAWAGTHGGRVRSWLHRYGALLFRDFHVAQEDFGEVVRALAGEPLAYVERSSPRTEIGDHIYTATDHPADQPIALHNENSYQREFPATLAFCCAVAAESGGATPVADTRRILARLDPGVVRDFARVGVRYVRNYGEGVGLPWPEVFQTDNRDEVAAYCQERGIDLEWKPDGGLRTSHVRPALAEHPVTGERVWFNHAIFFHVTSLLPEVRDAVLRQFAEEDLPSNTYYGDGRPIEPDVLDHLRDAYRSELVALPWEPGDVMVVDNLLSAHGREPFTGARRVLVGMAGTVRWDDVRVTGETP

SEQ ID NO: 18 - DNA, 1044 bp, Artificial Sequence
  Synthetic polynucleotide
atgtccgttcacagaggccccgtctccgggcagcgccgcgccatagcacttgctacgactgattgggttaggccggggggcacgccaccgggtagggaattgcccttggttctgcggccctcgcacgatgacgtggatcttgctgcgtgggctggtactcacgggggtagggttaggtcttggctccacaggtatggggcgcttctttttagagatttccatgtcgctcaagaagacttcggagaggtggttcgcgcccttgctggtgaacctctggcatacgtcgaaagatcatctcctcgcacagagattggtgaccatatatatacagcaacggatcatcccgctgaccagcccatagctttgcacaatgaaaattcataccagagagaattcccagcgaccttggcgttttgctgcgctgttgcagctgagtctgggggcgcgacaccagtcgcagatacccgcagaatccttgcacgccttgatcccggagttgtgcgcgattttgcccgggtgggggtgagatatgtccggaactacggtgaaggcgtcgggctgccctggcccgaggtgtttcagacagacaatcgcgatgaggtcgcagcctattgtcaagagcgggggatcgatctcgaatggaagcccgatggtggccttagaactagccacgtcagacctgctcttgccgagcatcccgtcaccggtgagagagtgtggtttaatcacgccatatttttccatgttacgtccctcctgccggaggttcgggatgcggttttgcggcagttcgccgaggaagacctgcctagcaacacgtactatggggacggccgccccattgagcccgacgtgcttgaccatctgcgcgatgcttatcgctcggagcttgttgcattgccttgggaaccgggcgacgttatggtcgtggacaacctccttagcgcacacggaagggagcctttcaccggtgcacggcgcgttctcgtcggtatggcgggtacagtgcgctgggacgatgttcgcgtgaccggagaaacccct

SEQ ID NO: 19 - protein, 334 aa, Artificial Sequence
  Synthetic polypeptide, QYD10, D10
MTVEPRREPGKPPIFAAPRGLDLADSCAWLRATLPELRAVLDSDGALLVRGLAFESAADFGHLRDAVISSRTPYRERATPRSSFGDDVFSSTDFPVDQRIMLHNENSYTLSFPGTIMFGCLTAPESGGATPVADCRKVLAALPDELVRRFREVGWGLVRNYTEYFGLGWQTAFGTDDPDEVLRYCAANDIGACWEANGRLRTVQRRSALIRHPRLDHEVWFNHVAFWSVWSLAEELRAYLLDDLAADELPYNTMFGDGKPLTEQDVEALARAYDAATLRETWQAGDVILVDNLLAAHGRDPYRGDRMILVAMGEPVDLTDCAPTVPPAPTLDSR

SEQ ID NO: 20 - DNA, 1002 bp, Artificial Sequence
  Synthetic polynucleotide
atgacggttgagccccgcagggagccagggaaaccgcctatctttgccgctccccgcggtctcgacctggcagactcttgtgcctggctccgcgccacactccccgaattgcgggcggtcttggattctgatggagcccttctggtccgcggacttgcgttcgagtcggcggccgacttcggacatcttagggatgcagtgatttcttcgaggacaccgtatcgggagagagcgacacctagatcttctttcggtgatgatgtcttctcaagcacagatttccccgttgaccagcggatcatgctgcacaatgagaattcttatactttgtccttcccaggcaccattatgttcggttgcctgactgcgcctgaaagcgggggagccacaccggtggctgattgtagaaaggttctggctgcgttgcctgacgaactggtccgcaggttccgcgaagttggttgggggcttgtcaggaattatacagaatattttggtcttggttggcagaccgctttcggaacagacgatccggacgaggttttgcggtattgcgcggcgaatgatattggcgcttgttgggaggctaacggacgcttgcggacagtgcaaaggagatctgccttgattcgccatccaagactggatcacgaagtctggttcaatcatgttgccttttggagcgtttggagcttggctgaggaactgagagcatatcttttggacgacctggccgctgacgaactgccatacaacacgatgtttggagatggcaaaccacttaccgagcaagacgtcgaagccctggcaagagcgtacgatgctgcaactttgcgggagacgtggcaggctggagacgtcatactcgttgacaatctgctggcagctcacggtagagatccttaccggggcgacaggatgattctcgttgcgatgggtgagccggtggatcttacagattgcgcccccacggttccaccggccccgacacttgactcgaga

SEQ ID NO: 21 - protein, 328 aa, Artificial Sequence
  Synthetic polypeptide, QYD11, D11
MTGSVTLTPLGGIIPRPRGEGLTTGAEYDLGPLGDAGPDWVRAHGPRLRERLATDGLILLHGLPTDGDGVDGFHDVVGSVGGDPLPYTERSTPRSVVKGNIYTSTEYPADQPIPMHNENSYAAHWPSTLYFFCHTAPDTGGATPIADGRAVLDLIPAEVRRRFSQGVVYTRTFRADMGLSWQEAFQTEDRGDVERHCRAHGQEFSWDGDVLRTRHHRPATAVDPGTGAEVWFNQAHLFHPSSLDPDLRQVLLETYGENGLPRDALFADGTPIPDADLATVRAAYTRAALALPWREGDIMLVDNLRMAHGREPFTGERRVLVAMTSADS

SEQ ID NO: 22 - DNA, 984 bp, Artificial Sequence
  Synthetic polynucleotide
atgaccggaagcgttactttgactccccttggaggcataattccccggcctcgcggagagggtcttactacgggagcggagtatgatctcggaccattgggagacgccggtcccgattgggtgagggcacacggccctaggctcagagaaaggcttgccacagatgggctcattcttctccatggcttgccgactgatggcgatggcgtcgacggctttcacgatgttgttggttccgttgggggtgatccactcccatacaccgagagatcgaccccgaggagcgtggtgaaagggaatatatacacgagcactgaatatccggcagatcaacccattcccatgcataacgaaaatagctacgccgctcactggccgtccactctttattttttctgccataccgcgccagatactggtggtgccaccccaatcgccgatgggagagctgttcttgatctgatccccgcggaggtccggcggaggttctcccagggggtggtttatacacggacatttcgcgctgacatggggttgtcttggcaagaagccttccaaacggaggaccggggagacgtggagagacattgtagggcccatggacaagaattctcgtgggatggggacgttcttaggacgagacaccatcgcccagcaactgcagtggaccctggaacgggagctgaggtgtggtttaaccaagctcatttgtttcacccatcgtccttggatcctgatttgaggcaggttctgctcgagacgtacggagaaaatggcctcccgcgcgacgcgctctttgcagacgggactccgatacctgatgctgatctggcaaccgtgcgggctgcttacacaagggcggcattggctctgccttggagagagggtgatattatgttggttgataatctcagaatggcgcatggaagggaaccctttacaggagagcggcgggtcctggtcgccatgacttctgccgactcc

SEQ ID NO: 23 - protein, 376 aa, Artificial Sequence
  Synthetic polypeptide, QYD12, D12
MACWRPGCAPSRSTGTPSCATPPSANSPNPAPPAQSPAMPSEPLTYTYSGEPILPFIERHAAQIQEGLLQHGAVRFKGFGITDASTFERAAKAIDPELKDDYLGTSPRNRMSGYVFSASELPPHFPIMQHCEMSFLPHAPRKLFFWCRVAPPEGGETPICDFAAVYDGLNPDIRREFEDKGITTLRNYAGPGQKGKSLKQLKPWPDMFLTTDKNRVNAICREHELEPHWLPGDALRLINTRPAIKVHPIHGKKVWYNHTQVFHSASAQLEYSHIASHQKTLRGYGLKLFLKLNDLWESASQRPEERAMHVTFGNGEEIPRAYVSHLMDVIWKNLHIAPWQAGDMIAIDNYRTAHGRLPYKGPREVNVCWAADTAAR

SEQ ID NO: 24 - DNA, 1128 bp, Artificial Sequence
  Synthetic polynucleotide
atggcgtgttggagacctggatgcgccccttcaagatctaccggaaccccttcatgtgccactcctcctagcgctaactctccaaatcctgcccctccagcgcagtctccagcaatgccatctgagccgctgacttacacctattccggcgagcccatcctgccgttcattgagcggcatgcggcgcagattcaagaaggtctcttgcagcatggcgcagtgcggttcaaaggatttgggataacggacgcgtctacgtttgagagggccgccaaggccatcgacccagaattgaaggatgattatctgggaacgtcccccagaaatagaatgtcaggttacgttttttcggcatcggaattgccaccgcatttccccataatgcagcactgcgaaatgtcatttctgccacatgcaccacggaaacttttcttttggtgcagggtggccccaccggagggtggcgagacaccgatttgtgactttgctgcggtctacgacggactcaatcccgacatacgcagagaatttgaagataaagggataactaccttgcgcaattacgcagggccgggccagaagggcaaaagcctcaaacagctcaagccctggcccgacatgtttcttaccactgacaagaatcgcgtgaacgcgatttgccgggaacacgaacttgagccgcattggctgcccggggatgcgcttagactgattaatactcgcccagctattaaagtgcatccaatacatggtaagaaagtttggtacaaccatactcaggttttccactctgctagcgcgcaattggaatactcacatatagcaagccaccagaagactctcagaggctacgggcttaaattgtttctgaaactcaatgacctttgggagtcggcctctcaaaggccagaggaaagagccatgcacgtcacctttggaaacggagaggagataccgagggcttacgtgtcacaccttatggacgtcatatggaagaaccttcatatcgcgccgtggcaggcgggagatatgatcgcaattgataactacagaaccgctcatggacgcctgccctacaagggacctcgcgaggtgaatgtctgctgggctgctgacaccgcagcacgg

SEQ ID NO: 25 - protein, 385 aa, Artificial Sequence
  Synthetic polypeptide, QYD13, D13
MQSNKQDGTADPTRFLTVWTDPKEGESISVSYVWLRDHCQCEECYNVATFQRSTSILDLPLDIQPALCNVTDSKIEIVWQDGHLSSYQLRQVVDNHVDNYRKRLEERRAQLVLWDRALIAAAEHSPSYARVTLNDLLCEDSVMRQVVGSLITYGFAFITKVPPNQQSTEMAVKRIFPVQRTLFGDMWTFSESKMDHSDTAYTNGFLGPHTDNTYFSDAAGLQVLHCIQFNGRGGETLLVDGYAAAERLRESDPAAYDRLCEYPLEAEYIEEDKHHRYMAPVIKRHRVLTGAVEQIRFNPYDRAPMRTLAAAEVPQFYADYRKLAEELYREEVQWKFQLTPGTVLIFDNWRVLHGRMAYSGKRVMTGCYVARTEYQSVARTMGLIE

SEQ ID NO: 26 - DNA, 1155 bp, Artificial Sequence
  Synthetic polynucleotide
atgcagtctaataagcaagacgggacagcagatcctacgcggttcctcacagtctggactgatcctaaagaaggggaatccataagcgtgtcatatgtctggcttagagaccactgccaatgcgaagagtgctacaacgtggcgacttttcagagatctactagcatcctcgatctcccattggatatacaaccggcactgtgcaatgtgaccgattccaagattgagattgtctggcaagacggacacctctcgtcataccagctgcgccaagtcgtcgataaccacgttgataactacaggaagcggctggaagaaagaagggcacagcttgttctttgggatagggcgcttatagctgctgccgagcactcaccctcctacgccagggtgaccctgaacgatctcctgtgtgaagactcggtgatgcggcaagttgttgggtcactgataacgtatggttttgccttcatcactaaggtcccaccgaatcagcaatcaacggaaatggcagtcaagcggattttcccggtccaaagaacattgtttggcgacatgtggacgttttcagagtccaagatggatcattccgacacggcctacactaatggcttcctcggcccgcacacggataatacgtatttctcagatgccgctgggctccaggttttgcactgtatccaatttaatgggagaggcggggaaacgctgcttgttgatggttacgcggcggctgaacggttgagggagtccgaccccgcagcatacgatcggctttgcgagtatccgctcgaagccgaatatatagaagaggataagcatcataggtatatggcccctgtgatcaagagacatcgcgtgctgactggtgcggtcgagcagatcaggttcaacccatatgatagagcaccgatgcgcacattggccgcagccgaagtcccccagttctacgcggattatagaaagctcgcggaagaactttatcgcgaagaggttcagtggaaattccagctcaccccaggtaccgttttgatttttgataattggagggttcttcatggtcggatggcgtattctggaaaaagagtcatgacagggtgctacgtcgcccggacagagtaccagtcagtcgctagaactatgggcctgatcgaa

SEQ ID NO: 27 - protein, 358 aa, Artificial Sequence
  Synthetic polypeptide, QYD14, D14
MTTTKTKSFITIKNKRFHYIWLRENCPSCRYAAPYQQLYDPNISDRPEYPQPLSVELNEETLTIDWDETPAHRSVFSVEWLLKNSYDPQPELESDNYIFWERAKLELKPPQTYNAETINNDGWMEQLFSLGFVILENIPPEKLESFLSSIGPIFNADYGTIMPLETRDKTTESLPSRDGCPLPPHHDLSYWGGHRLVEFLYCVENQNSGGESTLVDGFQVAQDFSQDYPQYYQTLLETPVQFWLVDKTHQYRFCNIATILECDRYGNLTTVRFSKRNCRPHLPFEQLEDFYQAYHTFFHYLKKNDYKHQFQLRSHNCLLFQNFRILHGRTAFDPALGKRKLNSGYVDWNFFVGRRNFQ

SEQ ID NO: 28 - DNA, 1074 bp, Artificial Sequence
  Synthetic polynucleotide
atgacaacgaccaaaaccaaatcttttattaccattaaaaataagcggttccactacatctggcttcgcgaaaactgtcccagctgtaggtatgcggctccttaccagcagctgtatgatccaaatatatcggatcggccagaatatcctcaacctttgtcggtcgagctcaatgaggaaacgcttacaattgattgggatgagactccagctcatcgctcggttttctcagtcgaatggcttctgaaaaattcttatgatcctcagccagaactcgagtccgacaattacatcttttgggaaagggccaagctcgaacttaaaccgccgcagacctacaatgcggagaccatcaacaacgacggatggatggaacagcttttctcactgggcttcgtcatcttggagaacatcccacccgaaaaacttgagtcctttttgtcttctatagggcctatcttcaacgcagactatggtaccatcatgccgttggagacacgcgacaaaaccaccgaatcacttccatcccgcgatggctgcccactgcctcctcatcacgacctgtcgtattgggggggtcaccggttggttgaattcctgtactgtgttgagaatcaaaactctggaggcgaatctacgcttgttgatggcttccaggttgctcaggacttttcccaagactaccctcaatactaccaaaccttgctcgagactccggtgcaattttggttggtcgataaaacccatcaatatcggttttgcaacatagcaaccatcttggagtgtgatcgctatggcaatctcactacggtccggttttcgaaaagaaattgccgcccccatctccctttcgaacaacttgaagatttctaccaggcctaccataccttctttcattacctgaagaagaacgactacaaacatcagtttcagcttcggtcacataattgcctccttttccaaaattttcggatcctccacggtagaacagcattcgaccctgcgctcggaaaacgcaaattgaatagcggctacgttgattggaacttcttcgttgggcggagaaactttcaa

SEQ ID NO: 29 - protein, 387 aa, Artificial Sequence
  Synthetic polypeptide, QYD15, D15
MACTIQKAEALDGAHLMQILWYDEEESLYPAVWLRDNCPCSDCYLDSAKARKLLVEALDVNIGIKGLIFDRKKVYITWPDEHYSEFQADWLKKRCFSKQARAKLQRELFFPECQYWGSELQLPTLDFEDVLRYDEHAYKWLSTLKKVGIVRLTGASDKPGEVSKLGKRMGFLYLTFYGHTWQVQDKIDANNVAYTTGKLSFHTDYPALHHPPGVQLLHCIKQTVTGGDSEIVDGFNVCQKLKKNNPQAFQILSSTFVDFTDIGVDYCDFSVQSKHKIIELDDKGQVVRINFNNATRDTIFDVPVERVQPFYAALKEFVDLMNSKESKFTFKMNPGDVITFDNWRLLHGRRSYEAGTEISRHLEGAYADWDVVMSRLRILRQRVENGN

SEQ ID NO: 30 - DNA, 1161 bp, Artificial Sequence
  Synthetic polynucleotide
atggcatgtacgattcagaaggccgaagctctcgacggagcacaccttatgcaaattctgtggtacgatgaagaagagtctctttacccagctgtctggttgagggataattgcccctgcagcgattgttatttggattctgctaaagccagaaagttgctggtggaagccttggatgtgaatataggaattaaaggattgatctttgatagaaagaaagtttatattacatggcctgacgaacattatagcgagttccaagccgattggcttaaaaaacggtgtttctcgaagcaagcccgggcgaaactgcagcgggaattgttctttcctgaatgtcagtactggggctctgaacttcagcttccaacgttggatttcgaggacgtccttcggtacgatgaacatgcatataagtggctttctaccttgaagaaagttgggattgtcaggctgacgggcgcttcggataaaccaggcgaggtgtcaaaactcgggaaaaggatgggtttcctttatctgaccttttatggtcacacctggcaagttcaagacaagatcgacgcgaataatgtggcgtatactacgggaaagctctcgttccatactgattatcccgctctccaccatccgccaggtgtgcagctgctccactgtatcaagcagacagtgaccggaggggatagcgaaatcgtcgacggctttaacgtgtgccaaaaacttaaaaagaataacccgcaagcctttcagatcctttcaagcacttttgtggactttacagacataggagtcgactattgcgacttctccgttcagagcaagcacaaaatcatcgaattggacgacaagggacaagtcgttagaattaacttcaataatgccacccgggatacaatctttgacgtgcctgtcgaaagggtgcaacctttttatgcagcgttgaaagagtttgtggatttgatgaactcgaaggaatcgaagttcacatttaaaatgaacccaggtgatgtcataacgttcgacaactggcggctgctccatggacgcagatcgtatgaagcgggtacagagatttctcggcatctggaaggagcgtatgcagactgggacgttgtgatgtctcggttgaggatcctgcggcagcgggttgagaacggaaac

SEQ ID NO: 31 - protein, 543 aa, Artificial Sequence
  Synthetic polypeptide, QYD16, D16
MSRLLRTAFRLPRASFPPASVLSRRVYSSSVVRRSAANAIPPSNNVAPTSSDAAPTASSVASAASIAAAEPSASHEIADASNDPASSQSNQSSPPEAGDQHDDKTQTTSAGTVEYQDDKYTIRRKEGGLVIEEGRKRPIEILDIILRDSCKCSSCVDPHSKQRNFRTSDISSDIISENPTIKHGKLHVTWKNDFRGAPAESAHESTYDLRQLRHPIISPVEMESAGKNRWRAYWDNSRMQHWQHWITYDQFINNDISFAWSMRTLAELGLIFIKDIPDSREMVEKIATRMGPIRDTFYGRTWDVRSIPQATNVAYTDQFLGFHMDLMYMNDPPGYQLLHCLQNSCEGGESLFVDTFRVAYDMKQDDHKNYSRLLHHHIPYHYNHPDHFYTNSWPVFETETFDNSVTEGTNFSKSRLVHVNYSPPFQAPRKVQSPVPRKFREKNEALAKFASLLEDERYMFELKLNPGECVVFENRRVAHARRGFKTSTGERWLAGAYVDEDAMLSGFKTSSNKYPNLWRLKRKGLNSKHVKMLKGVNKPDELP

SEQ ID NO: 32 - DNA, 1629 bp, Artificial Sequence
  Synthetic polynucleotide
atgtcaagactcttgagaactgccttcagattgccgcgcgcctctttcccacccgcatcagttctgagccggagggtctactcctcgtccgttgttagaagaagcgctgccaacgcaatcccaccatccaacaatgtcgccccgacatcttcagatgcagcgccaacggcgagcagcgtcgcatctgcggcgtcgatagccgcggcagaaccgtccgcttcgcacgagatagccgatgctagcaatgacccggcgagctcgcagtcaaatcagtcgtcgccaccagaagccggagaccaacacgatgacaaaactcaaaccacctccgcagggaccgtggaataccaggacgacaaatatacaataagaaggaaagagggcggccttgtcatcgaggaaggcaggaaacgcccaattgaaatcttggacataatcctgcgggactcctgcaaatgctcgtcgtgtgtcgatccgcactctaaacaacggaattttcgcactagcgatatatcttcggacatcatttccgagaatcctactattaagcacgggaaactccacgttacctggaaaaacgacttcagaggggcccctgcggaatctgcgcacgagtcaacttatgatctccggcagttgcgccatccgattataagccctgtggagatggagtccgcagggaaaaaccgctggagagcgtattgggacaattcacggatgcagcattggcagcattggataacatacgatcagtttatcaacaacgatatttctttcgcgtggtcaatgcgcacccttgcagagcttggtctcatattcattaaagacattccagactcgagggaaatggtcgaaaaaatagcgactagaatgggtccaatccgcgacactttctacggacgcacttgggatgtccggagcattccccaagcgaccaacgtcgcgtatacggatcagttccttggttttcatatggatcttatgtatatgaatgacccgccaggttatcaacttttgcactgcctccaaaactcttgcgaaggaggagaatccttgtttgttgacactttccgcgttgcatacgacatgaagcaagacgaccataaaaattactcgagactgctgcaccatcacatcccctaccattacaaccaccctgaccacttctatacgaactcttggccggtgtttgagacggagacgtttgacaactcggtgaccgaaggtacaaatttttcgaagtctcgcttggtccacgttaactatagccctccatttcaggctcctagaaaggtgcagagcccagtgccccgcaagtttagagagaaaaacgaagcactcgccaaatttgcgagcctgcttgaagacgaaaggtatatgtttgagttgaaactcaatccaggagagtgcgttgtcttcgaaaatagacgcgtggcacatgctaggaggggctttaagacgtcaacgggagaacggtggctggcaggtgcttatgttgacgaggacgcaatgctgtcaggcttcaaaacctcctctaacaagtaccccaacctttggcgcctcaaacgcaagggtctgaactcgaaacacgtgaaaatgcttaagggcgtgaacaagccggatgaactgcca

SEQ ID NO: 33 - protein, 290 aa, Artificial Sequence
  Synthetic polypeptide, QYD17, D17
MNCASASNTPLKPWLAADLAESQQWIVAADPELALLSTEALQAWFAPARDQLLHGYGVVLIRGLSPQPESTFRRLYLALGRCLGTPDGTYGELYDVTDSGQSYLTKAIPVSQTRAATSMHTDSSRLETHPRWVGLACVQQAPVGGGSRLASALAVHNHLKASDPRSLERLQRSFARDVVTPGAVDPLALIAHNRFPVFSTDTDGPTLRYMRYWIEKGHQRLGQPLDANDLRAFDALDAALNHPRFCHSFQLQEGDILLIDNHKLVHDREAYEDDPHRPRRLIRLWLNQTP

SEQ ID NO: 34 - DNA, 870 bp, Artificial Sequence
  Synthetic polynucleotide
atgaactgcgcatcagcatctaacaccccacttaagccatggctcgctgccgacctcgctgagtcgcagcagtggatcgttgcagcagatcctgaattggccttgttgtcaactgaagcccttcaggcctggttcgcgcctgccagagaccagttgcttcacggctatggagttgtgctcatcagagggctctcacctcaaccggagtctacatttcgccgcctgtatttggctttgggtcgctgtttgggtacgcctgacggtacatacggagaactctatgatgttacagattcgggtcaatcatatctcacgaaagcgatcccggtctcgcagacacgggccgcgacatcaatgcatacagattcctccagactggagacacacccaaggtgggttgggttggcttgtgtccagcaagcgcctgtcggtggcgggtccagacttgcttcggccctcgccgttcataatcatcttaaggcgtctgaccctcgctcgcttgagagactccaaaggagctttgcgagagacgtcgttacgccgggggcggttgaccctcttgctcttatcgcccacaacaggttccctgtttttagcacggatactgacggcccgactcttagatatatgcgctactggatcgagaagggccaccaacgcctgggacaacccctcgacgccaacgaccttagggcattcgatgcgctcgacgccgcacttaaccatccgagattttgccattcgttccagcttcaagagggagacatattgctcatagataatcataagctggttcatgatagggaagcttatgaagatgatccgcacagacctagacgccttatcaggctctggctcaatcagacgccc

SEQ ID NO: 35 - protein, 399 aa, Artificial Sequence
  Synthetic polypeptide, QYD18, D18
MATLTQTSAPAPAPAQIIHSNAPRDIFPDGLKTTGQHPPIYEELHPFEDFPKSIEGRTLWKAEDYKDAPEKWTHRFSAEEVEELGATADAFLASGTPLTGISKSNFPLPKLSALLYELRDDLLNGKGFILFKGFPVQEWGNHKSAVAYMGLGTYLGYFVSQNSRGHVLGHVKDLGEDPTQIDSVRIYRTNARQYFHADDSDIVGLLCIARALEGGESDIVSTHHVYNTLAAERPDVLKTLIEPIWYFDRKGETSKGQEEYIRTSVIYLERGDNPRVYTKWDPYYVRSLTRFSDAGLIPPLSDRQIEALEVLEQTCQRLSLHMILEVGDIQFVSNSHVLHARTAYKDYAPPAPRRHLMRLWLSTPESEGGWRLPFWDSNEKKRGGVQVDDTPPVALLDAE

SEQ ID NO: 36 - DNA, 1197 bp, Artificial Sequence
  Synthetic polynucleotide
atggccacactcacgcagacttccgcaccggcgcctgcccccgcgcaaattatccattcaaatgcccccagggacatattccctgatggacttaagacaactggtcagcaccccccaatatatgaggaactccaccccttcgaggattttccaaaaagcattgagggtcggacactctggaaggctgaggattataaagatgctccggagaaatggactcacaggttttctgcggaggaagttgaggagttgggggcaaccgctgacgcatttctcgcctccggtacacctcttacaggcatttctaagtcaaacttccctctgccaaaactgtccgcattgctttacgaactgcgggatgatctcctgaacgggaaaggatttattctctttaagggttttcctgtgcaggagtgggggaaccataagtctgcagtcgcgtacatggggctcggtacctacctggggtactttgtctctcagaactctaggggccatgtcttgggacacgtgaaggacctgggggaggaccccacccaaatcgactccgtgagaatctatcgcactaatgcaaggcagtacttccacgcggacgactcggatattgtcgggctcctgtgcatagcaagggcccttgaagggggggaatccgacatcgtgtccacgcaccacgtgtataacacactggcggctgagaggccagatgttttgaaaactttgattgagccaatttggtactttgatagaaagggagaaacctctaaagggcaggaggagtatatacgcacatctgtcatctacctcgagcggggagacaatccgcgcgtttacactaaatgggacccctattatgtcagatcgcttacgcggttctctgatgccggactcatccctccgctttcggacaggcagattgaagccttggaggtgctcgaacaaacctgccagcgcctgagccttcacatgatcttggaggtgggtgatatccagtttgtgtcaaactcccacgtccttcacgctagaacagcttacaaggattatgctcctcccgcaccgcggcgccacctgatgaggctgtggctgagcacacctgagagcgaagggggatggcggcttcctttttgggactccaatgaaaagaagagaggaggggtgcaagttgacgataccccgcccgttgcccttctcgacgctgaa

SEQ ID NO: 37 - protein, 406 aa, Artificial Sequence
  Synthetic polypeptide, QYD19, D19
MSTHTLSPKSSNDSLQSRFSAASSPDKLAPALAGPMVWEKLDPSDYVVDLSPREVQDIRAAVIKVKIAGTPRAEIRQETFVLDNPVLARKLSSVSEELHQGQGVVVLRGLDAANFNDEEAVVAFAGVCAYVCPERATDSYANQTLSMLPSLITLAETPSNFFTGHVRDATKEAVPTWAKDIGLAGSKITAAMDFHSDRFSGDVIALHVRNDGGIGSGGEQYVASFWRIYNELLELDPEVLETMAEADWPFELKQKDKEPYLELGPTLFFSKGKPMCQLVKAPLLGTPLIHRDPSMPSVSDKQMHAMHAVGELAKRFCTKLDRKQGDIQFINNLSVMHAREAYRGAQGKPSTRHLLRMFLRDPQNAWEKPARFRNNFDDPFTPGRPQNLPVIDNDPWRIISGRESHG

SEQ ID NO: 38 - DNA, 1218 bp, Artificial Sequence
  Synthetic polynucleotide
atgtccacccacaccctttcgccaaaatcctcgaacgacagccttcagtcaagattctctgcagcctcgtcgcccgacaagctggcgcctgcactcgccgggcccatggtgtgggagaaattggatccatcggactatgttgtcgacttgtctccgcgggaggtgcaagacatccgcgccgcggttataaaagtgaaaatagctggaacgcctcgcgctgaaattaggcaagaaacttttgtgttggataaccccgtccttgccaggaaattgtcctcggtgtctgaggaactccatcaaggccagggtgttgtggtgcttaggggtctggacgcggctaattttaacgatgaggaggctgtggttgcgttcgctggtgtctgcgcctacgtgtgcccagagcgggccaccgacagctacgcaaaccagaccctctctatgctgccatccctcattaccttggcggagactccatccaactttttcaccgggcatgttagggatgcaacaaaggaagccgtgcctacttgggccaaagacatcggcttggcaggatcgaagattactgctgcgatggactttcattctgacaggttcagcggggatgtgattgctttgcacgttaggaatgatgggggtataggctctggtggcgagcaatatgtcgcatctttttggcggatctacaacgagctcttggaacttgaccctgaggtgctggaaacgatggcagaagcagactggcctttcgagttgaaacaaaaagataaagaaccgtatctggagctcggaccaactcttttcttctcaaaaggcaaaccgatgtgccagctggttaaagctcccctcctcgggacgcccctgatacatcgcgacccatctatgccatcggtctctgacaagcagatgcacgctatgcacgctgtgggggaattggccaagaggttttgtaccaaattggaccggaagcagggcgacatacagttcatcaacaatctttctgtcatgcatgcccgggaggcgtatagaggggcgcaaggaaaaccgagcactagacatcttctgcggatgttcttgagggatccccagaacgcatgggagaaacccgctagattccgcaacaacttcgatgatccgtttacgcctgggagaccacaaaatttgccagtgatagacaacgatccctggcggataatttctggccgcgagtctcatggc

SEQ ID NO: 39 - protein, 356 aa, Artificial Sequence
  Synthetic polypeptide, QYD20, D20
MSTPSPNSILKDEYPKTITVKELHPTFGAEVLGIQWGDNGVISDEQLQELRDTYGFIILRATPLTDSTHVSFSRLFASGPLDDISRFLPPGRVPRYYPHLELFDASNLSDDGRAILDPSSSPRAMLLRANSSWHSDLAYNPRRSSYSLLRAVELPSREPGEMGEIEGNTEFADSRTAWEELAAEKKRELLTKDWTGVYNAAHSRKLGAPEYFKDVNPEEGPIARHKVVQEHVESGRMNLCVGAYLWRLEDGEGKTVPGSEGIIKFLNEHVANKSFVASVRWERPGDLVIWDNRAVLHRAGEFKGVGKHRRDMRRTTAFDQGPTAWGLNGEGAPLPTLESITKEKGLPLTQSAAVER

SEQ ID NO: 40 - DNA, 1068 bp, Artificial Sequence
  Synthetic polynucleotide
atgtcaacgccctcacccaacagcatactgaaagatgagtatccaaaaactatcacagttaaggagttgcatcctactttcggcgctgaagtcctgggcatccagtggggcgataacggagttatctcggacgagcaactccaagagttgcgggatacatacggttttataatacttagggccacgcctctcactgattcgacccacgtttctttctcaagactgttcgcgtctgggccgctggatgatatttcgagattcttgccccccggacgcgtgccaagatactacccccatttggaactcttcgacgcgtcaaatttgtccgacgacgggcgcgctatacttgacccgtcttcaagccctcgggcaatgctgttgcgcgccaattcctcatggcatagcgatctggcgtacaacccgaggagatctagctactcccttctcagggccgtggagttgccatcgcgggaaccaggcgaaatgggagagatcgaagggaatacggagtttgcagacagccggaccgcctgggaggaactggcggccgagaaaaaacgggaactgctcaccaaagattggactggagtctacaacgcagcccacagcaggaagctgggcgcaccagagtattttaaggatgtgaacccggaagaaggccctattgctagacacaaggttgtgcaagaacatgttgaatcaggtagaatgaatctttgtgttggagcttatctttggcggctggaagacggtgaagggaagaccgtcccagggagcgagggcataatcaaatttttgaatgagcacgtggcaaacaagagcttcgttgcatcggtgcggtgggaacgcccaggcgacctcgtcatttgggacaatcgggccgtcttgcatagggcgggagaattcaaaggtgtcgggaagcacaggagggatatgagacggacaaccgcttttgaccaggggccaacagcttggggccttaacggagagggcgcaccccttcctacgctcgaatcgataacaaaggagaaaggcttgcctctgacccagagcgccgctgtggagagg

SEQ ID NO: 41 - protein, 294 aa, Artificial Sequence
  Synthetic polypeptide, QYD21, D21
MTTIETKPLTPCFGVEVSGMTLSEVSKGHDFARLRDLFEEHSALLFRAQEISNEEHMALAQLFGPIEDRKADERKPGEAFEVPEVSNVQKDGTTSGEMDLHTLNLKSNFLWHSDSTFLPTPALTNILIGRVVTTEGGATELASTRAAWAAMPEELKSRIRGRGIWHRYSHSRRKISPELAKLPMFNKWPDQHWSAVWPNPVNGREGLYIASHAFKVDGYDEAESQELLDELMAFCTQPEFTYAHQWNVGDVLIWDQRAVLHRGTPWPYEQPRTLSSICVSVTEADGIGSIRMPA

SEQ ID NO: 42 - DNA, 882 bp, Artificial Sequence
  Synthetic polynucleotide
atgacgactatcgagacgaagccgctcactccatgctttggcgtcgaggtttctggcatgacgctgtctgaggtttccaagggccacgattttgctagattgcgggacttgtttgaagagcattctgcccttctgttcagggctcaggagatttcgaatgaggagcatatggccctcgcgcaactgtttggtccgatagaggatcgcaaggcggatgaaaggaaacctggagaagcctttgaagttcctgaggtgtcaaacgtccaaaaggacggaactactagcggtgaaatggatctccatacgctcaacttgaaaagcaactttctttggcattcggattctaccttcctcccgacccccgctctcacgaatattctcatcggaagagtggtgacgacagaagggggtgcaacggaacttgcttcaacaagggcggcatgggccgccatgccagaggagttgaagtccagaatcagggggaggggtatatggcatcggtactcccattctcgcaggaaaatatcgcctgagctggcgaaacttcctatgttcaataaatggcctgaccagcattggagcgccgtgtggccgaaccccgttaatggtagggaaggcctctatattgcaagccatgcatttaaggttgacggatacgatgaagcagagtcacaagaacttcttgacgagttgatggcattctgtacccagcctgaattcacctatgcgcaccaatggaatgttggagacgtgctcatatgggatcaacgggctgtcttgcaccggggtaccccgtggccttacgagcagccccgcacattgtcgtccatttgcgttagcgtgacagaagcagacggtatcgggtctatacgcatgccagcc

SEQ ID NO: 43 - protein, 368 aa, Artificial Sequence
  Synthetic polypeptide, QYD22, D22
MAPAATTTPGVSWEAKGFKAVDKTKTIRVQEDGLKYPEYYPYNDPNDKFPDPEPFDYHERALDADPSLPVFYNDHVTVEEITPFIGVKVTGLDLASLDKAGQDQLALLAARKGIVFFASNDKVKQTYRDTSMERKLEMARYYVQPRPPTSTEISVVYQDKVNTVRKHWWPNRLTNAIWHIDQTQERQPPGITFFCCMQHDAPSGGDTLVGSLVEAYNRLSPKMKEFVCGLKAVHSSAVMSAKAARVGGASRRNEIESLHPLVTVHPATGSKSLYINPERMTYIEGLRNEESDNMLKFLSDHVKLGADFHARYKWSEGDVCVWDQRVIIHSAVLDNMTHRRHFIRITALSSVPIQATYDPKDEENDEEE

SEQ ID NO: 44 - DNA, 1104 bp, Artificial Sequence
  Synthetic polynucleotide
atggccccggccgcgacaacgacacccggcgtttcttgggaagcgaagggattcaaagcggtcgataagactaaaacaattagagttcaagaagacggtcttaaataccctgaatactacccctataatgatcccaatgataagtttcccgacccagaaccattcgactaccatgagcgggccctcgacgcagatccttcgttgccggtcttctacaatgaccatgttacagtcgaggagatcactccgttcattggcgtcaaggtgactggactcgaccttgcctccttggataaagcaggacaagatcagctcgcccttcttgctgctcgcaaagggatcgtgtttttcgcctctaatgacaaagtcaaacagacatatagggacacttcgatggaaaggaagcttgaaatggctaggtactatgttcaaccgcgccctccgacttctacagaaatttctgtcgtgtatcaggataaggtcaatactgttcggaagcactggtggcccaatcgcctcacgaacgctatatggcacatcgatcagacgcaggaaaggcaaccgcctggcataactttcttctgttgtatgcaacatgatgcgcccagcggaggagatacgctggtcggaagcttggttgaagcttacaacaggctgtctcctaagatgaaagaattcgtgtgcggactcaaggccgtgcattcaagcgccgtcatgagcgcaaaggctgcccgggttggaggcgcttctcgcaggaatgaaatcgagtcccttcaccccctcgtgaccgttcacccggcaacgggctccaaatcactttatattaacccagagaggatgacgtacattgagggactccggaatgaggaaagcgataacatgttgaaattcttgtcagatcatgtcaaacttggtgcagattttcatgctagatataagtggagcgagggtgatgtttgtgtctgggatcaaagagtcattattcattccgccgtccttgataacatgacccacagaaggcattttattcggattactgccctgtcatcggttccaatacaagcaacttatgaccccaaggacgaagaaaatgatgaggaagaa

SEQ ID NO: 45 - protein, 312 aa, Artificial Sequence
  Synthetic polypeptide, QYD23, D23
MSFYRDFNDKLAIQAWNGRKREPYETIEVRQLGPTVGAEIGGVDLAKDVSEAQFAEIHRALDENLAIVFRDQDISKEDHKRFARRFGKSLHRHELAATRFRHDGPFDPEFLSWKTDANSRFTAGDGWHPDVSCDPSPIAVSLLRVTKTPPLGGDTAFANMYLAYEFLSDPIKQLLDGLTAIHDGSLAWTAGYGAKPDPGKTYPQSEHPVVVTHPRTGRKFLYVNASFTSHIVQLTRRESDALLQLLFRHVESQLALQTRVQWRPNSLLVWDNWASQHHAVWDYYPEERWGERVSVVTGEVPQARLDFSEAAE

SEQ ID NO: 46 - DNA, 936 bp, Artificial Sequence
  Synthetic polynucleotide
atgtcgttttacagagactttaatgataaacttgccatacaagcttggaatggccgcaaaagggagccatacgaaactatagaagtgaggcagcttggtccgacagtcggggcagagatagggggtgtcgatctggcgaaggacgtgagcgaagcgcaattcgcagagattcacagggcgctcgacgaaaacctcgccattgtttttagggatcaagatattagcaaggaggatcacaaacgctttgctcggcgctttggaaagagccttcatagacatgaactcgcggccacccgcttcagacacgacggaccctttgatccggagttcttgtcgtggaaaacagacgcgaattcccgcttcaccgcgggcgacggatggcatccggatgtgagctgcgacccctccccaatagccgtctctcttctcagagttaccaaaaccccgccgctggggggcgatactgcatttgccaatatgtatcttgcctacgagtttttgtccgatcctatcaaacaacttctggatggtcttacggccatccacgatggctcattggcctggacggcaggctacggagcgaaacctgacccggggaagacgtatccacagtcggagcatccagttgtggtgacgcatccccgcactggtaggaaattcctttacgttaacgcatcgtttacgtcccacatcgtccagctcacacggcgggaatccgacgcccttcttcagctccttttccgccatgttgagagccaactggctttgcaaacccgggtgcaatggcgccccaattcgctgctcgtttgggataactgggccagccaacatcacgctgtgtgggactattaccctgaggaaaggtggggcgaacgggtgtcggttgtgacgggagaagtcccccaggctaggttggacttcagcgaagctgccgaa

SEQ ID NO: 47 - protein, 317 aa, Artificial Sequence
  Synthetic polypeptide, QYD24, D24
MYHYKHAKSVVEPVPIEQRYRHIRPRPQMPDFAAEVDIDLSRPLDEPVREELRQALRDFEVLFLTPQELTPEQHLELASVFGPVAQGAYFPRKDSHPQIEVLANDARRPASVDHWHSDLTWLEEPPAGVAIQLVEIPANGGNTAWASMSKAFAALSPGLQEYLRGLRATHTWEISQWRNYLANLGEEVLLNSIRRFKPVSHPVVQKHPQSGKEILYVNETFTRNIDDVPPQESREILRFLCEWLKQPEFVHSHRWQRNGIAIWDNLATQHYALADYWPHSRVNQRVTFNAQGSRAPVANTLAEVGGLGVESRVAYGY

SEQ ID NO: 48 - DNA, 951 bp, Artificial Sequence
  Synthetic polynucleotide
atgtatcactataagcatgctaagagcgttgtcgagcctgtccctatagagcaaagataccgccacatcaggccaagaccgcaaatgcctgactttgccgccgaggtggacatcgatctctcaagacccctggacgaacccgtgagggaggaattgaggcaagcgctgcgcgacttcgaagtcttgttcctcacacctcaggaactcacacccgagcaacaccttgaactcgcctcagtcttcggcccagtcgcacagggggcttatttccccaggaaagactcgcatccccagatcgaggttttggcaaacgatgcaaggagacctgcatcggtggaccattggcactctgatctcacttggcttgaggagccgccagccggggtcgcaatacagttggtggaaatcccagccaatggaggcaatacagcttgggcttcaatgtcgaaagcgtttgcagctctgtcccctggtctgcaggaatatctcagaggcttgcgcgccacccacacctgggaaatttctcaatggcgcaactatcttgcaaacttgggtgaagaggtgctgctcaactcgataagaaggttcaagcctgtctctcaccccgtggtccaaaaacacccacagagcgggaaggagatactctacgtgaacgagacctttaccaggaatatcgacgatgttccaccgcaggaatcgcgggagatccttaggttcttgtgtgaatggcttaagcagccagagttcgttcactcccatcggtggcagcggaatgggattgcgatctgggacaaccttgcgacacagcactacgccttggctgactattggccacattcgagagtcaaccaaagagtgaccttcaatgcgcaaggctctagggctccagttgccaatacgcttgctgaagtgggtggactcggagtggagtccagggtggcttatggatat

SEQ ID NO: 49 - protein, 288 aa, Artificial Sequence
  Synthetic polypeptide, QYD25, D25
MTTVFEAPVRSSLAVQPLQPTIGAEISGVDLREPLTPEVRDEIKANLLKYKVVFFRDQELTQEQHEAFARQFGPLYTHPGAAKADSGVTPAIHRIASEDFKKYEKAHTPQAGDDTWDPYHTDTSWRLVPTWGAVLRAVHLPEVGGDTVWVDAALAYQGLSDDVKARLEGHHVAHDYRAALHQSGHDYPVVAHPVVRVHRETGEKIAWVNFTQRPTILGLDRAESKELLTAVIDQYRKPANQVRFSWRPGSVAFWDNRATVHYAVRNYGTFPRLLERILIADETLYADL

SEQ ID NO: 50 - DNA, 864 bp, Artificial Sequence
  Synthetic polynucleotide
atgacaacggtgtttgaggcccctgtgaggtctagcttggcagtccagcccctccagcctacaattggtgctgagataagcggcgttgatttgcgcgagccactcacacccgaggtcagagatgaaattaaggcgaatttgctgaaatataaggtcgtgttcttccgcgaccaagaattgacccaagagcaacacgaggcctttgctcgccaattcggaccgctctatacacacccgggtgcagccaaggcagactccggcgttacccctgctatacatcggatagcttcagaagacttcaaaaagtatgagaaggcccatacccctcaagcgggcgatgacacatgggatccataccacacagacacgtcttggagactcgtgccaacatggggcgccgttttgcgggctgtgcatctgccggaggttggtggtgataccgtctgggttgatgcggctttggcgtaccaaggcctcagcgatgatgttaaggcgaggctggaagggcaccacgtcgctcatgattatcgggcagcactccatcaatctggtcatgattatccggtggttgctcatcccgtggtccgggtgcatagggaaactggcgagaagattgcttgggtcaactttactcaaaggccgacaatactcgggctggatcgcgcagaatctaaagaacttctgacggcggtcatagatcagtatcgcaagccggccaaccaagtgaggtttagctggcggcccggttcagtggcattttgggacaatcgggcaactgttcattatgccgttaggaattacggtacgttcccacgcctcttggagcgcatactgatcgcagacgagactttgtacgctgacctc

SEQ ID NO: 51 - protein, 298 aa, Artificial Sequence
  Synthetic polypeptide, QYD26, D26
MSEFIYTLPSHNRAASYSHIRVTPTTGAIGADVENLDLTRLSDEGFAELRQALLAHKVIFIRDQALSVADLERVTQHLGEFGREPYVTCMADHPHVVHVVKEASEKAPFVFGGAWHTDWTFQERPPAFTLLYGHDIPPYGGDTCYANLALAYEWLSPGMQALLENLDAVHSPEMAYGVNAKHNALLENMAINYGSDTSADERSHPLVTRHPETGKKVLFINPAYTTGIKGLRTEESRPLLDYLFKLATSEAFTCRMRWRQGTLAIWDNRSTWHMPVADYHGMRREMFRTTVIGEVPSR

SEQ ID NO: 52 - DNA, 894 bp, Artificial Sequence
  Synthetic polynucleotide
atgtcagaatttatctatacgctgccttcgcacaatcgcgctgcaagctactcgcatatacgggtgaccccaacaacaggggctataggcgccgacgtggagaacctcgatcttactcgccttagcgacgaagggttcgcggaactgaggcaggcgctcttggcacataaagtcatcttcattagagaccaagctttgtctgttgccgatctcgagcgcgtgacccagcatctcggcgagttcgggcgcgaaccttatgttacctgtatggctgaccatccgcatgttgtgcacgtggtcaaagaagcatctgaaaaagcaccgtttgtctttgggggggcttggcacacggactggacttttcaggagcgccctccggcattcaccctgctgtatggacatgatatacccccttacgggggagatacgtgctatgcgaatttggcgttggcttatgaatggctttcaccagggatgcaagctcttctggaaaacctcgacgcagtgcattctccagagatggcatacggagttaatgcgaagcataatgcgctccttgaaaacatggcaattaactacggttcagacacatctgccgacgagagatcgcacccactcgtcaccagacaccccgagactggcaagaaagttttgttcattaaccctgcatacacgaccggaataaaaggtctccggacagaggagagcaggcccttgctggactatctttttaagctggctacgtctgaagctttcacatgcaggatgaggtggagacaggggactcttgcgatctgggataatagatcgacttggcatatgccagtcgcggactatcacggtatgaggcgggagatgtttcgcaccacagtgatcggtgaagtccccagccgc

SEQ ID NO: 53 - protein, 308 aa, Artificial Sequence
  Synthetic polypeptide, QYD27, D27
MGYSVETLREGLGFGKQVVGLARQDIDREAVRAELRDHWIRDGLVVFRGSEVTPEFQIALSRVFGELEVHPIPELRSADHPELITLVSDKDKEGLFEVDGVESVAFLPWHSDLIFVDSINHGGLLTAKTIASWGGQTGFIDQVQAYELLPETLKAEIEGLEIVYQLCVNPGGSRYGTRSRVRTILTGDFEKSVGPRLDADYPPVVHPVVFVQPDTGRKVLNVSPFGALHILGHDDEAGHALLGRLVDHLTACPAYFHQWQPSEMLLWDNWRMAHCVTGAAPNEVRIMERTTIMGDYGLGRRLHGRKAA

SEQ ID NO: 54 - DNA, 924 bp, Artificial Sequence
  Synthetic polynucleotide
atgggatactcagtcgagacacttagagagggcttggggttcggcaaacaggtcgttgggctcgcacggcaggacattgacagagaggcagtgagggcagagttgcgcgatcactggattcgggacggtctcgtcgtctttcggggatcagaggtgacccctgagtttcagatagctctgtccagagtcttcggtgagttggaggtgcatccgatcccagagcttcgcagcgccgatcatccagagctgataacgctggtttccgacaaggacaaagaagggctctttgaagtcgatggggttgaaagcgtggctttcttgccgtggcattctgacttgatctttgttgattcaattaatcacggaggactcttgaccgccaaaactatagcttcgtgggggggtcaaaccggattcatcgaccaagttcaggcgtatgagttgttgcccgaaaccttgaaggcggaaatagaagggctcgagatagtctaccagttgtgcgtcaaccctggtggaagccggtacgggacccggtcgagagtgcgcaccatactgactggagacttcgaaaaatccgtcggacctcgcctggacgcagattacccaccagttgtgcatcccgtggtttttgttcagccggatacgggacggaaagtcttgaatgtgtctccctttggggcgcttcatatactcggtcatgacgacgaagcaggccatgccttgcttggtcgcttggtcgaccatcttacagcttgtcctgcctatttccatcaatggcaaccctccgaaatgcttctgtgggataactggaggatggcacactgtgtgactggggcagcgccgaacgaggttaggattatggaacgcacaacaataatgggagattatggccttggtcggaggctgcatggaagaaaggcagct

SEQ ID NO: 55 - protein, 283 aa, Artificial Sequence
  Synthetic polypeptide, QYD28, D28
MAYKTLSNALGTEVYGFDSPFPHDAEAVRALIEAWHAGGICRLRRQRLDMAEFVEFSRIFGRPERALNQERKLTSREDLPELMIVSNILENGVSIGHLGSKEAYWHTDMCYTDVPPIASILYAIEVPAHGGNTEFMNMYRVHDALPASLRRQIAGLSIKHDRSYTAVGELRYGFDSVVDVTTCPGSIHPIVRVHPVTQRPYLYLGRRLNAYVVGLPVGESEALLDELWRYTRLDGVTWTQRWEVGDIMIWDNRCTMHRRDAFDANARRLMWRTQIQADPARPL

SEQ ID NO: 56 - DNA, 849 bp, Artificial Sequence
  Synthetic polynucleotide
atggcctataagacactttcaaatgctctcggtacggaagtgtatggttttgactcgccttttccccatgacgcggaggcggttagagcacttattgaagcttggcacgcgggtggaatttgtcggcttcgcaggcagcgcctcgatatggctgagtttgttgaattctcacgcatatttgggcggcctgaacgggcccttaaccaagaaaggaaacttactagccgcgaggacttgccagaattgatgattgtgtctaatatattggaaaacggagtttccatcggacatttgggttccaaggaagcatactggcatactgacatgtgctatacggatgtcccgccaattgcgagcatcctgtatgcgatagaggtgccagctcacgggggaaacactgaatttatgaacatgtaccgggttcatgacgcattgccggcgtccttgcggcgccagattgctggccttagcatcaagcacgacaggtcatacacggctgttggagaactgcgctatgggtttgacagcgttgttgacgttacaacctgtcctggctcaatacatcccatcgtcagggttcatccagttacacaacgcccgtacctctaccttgggagaagacttaatgcttatgtcgttggcctcccggtcggagagtcagaagccctcctcgatgagctttggcgctacacgagactcgatggtgtgacttggacgcagagatgggaggttggtgacatcatgatttgggacaaccggtgtacgatgcacaggagggatgccttcgacgctaacgctcgcagactcatgtggcgcactcaaatacaagcagaccctgctcgcccgctt

SEQ ID NO: 57 - protein, 288 aa, Artificial Sequence
  Synthetic polypeptide, QYD29, D29
MSINSEYLHPLFVAEYEEADLQGALSPTEVRDVEHQMDKKAVLVFRGQPLDQDQQIAFARNFGQLEGGFIKVNQRPSRFKYAELADISNVSVDGKVADREARESVGNFANNQLWHSDSSFQQAAARYSMLSASVLPPLGGDTEFWDIHATNLGGRDDLPRELQGLRAERYLQNSRFILGDTDYSESQRNAMPPVSWPLVRTHAGSGRKFLFIGAHAGHIEGRPVAEGRMLLAELLEHATQRKFVYRHRWKVGDLVMWDNRCVLHRGRGYDITARRELRRATTVDDGVV

SEQ ID NO: 58 - DNA, 864 bp, Artificial Sequence
  Synthetic polynucleotide
atgtctattaattccgaataccttcatccgctgtttgttgctgagtatgaagaagctgatcttcaaggagcgttgtcacctacggaggtgagggatgtggaacaccaaatggataaaaaagctgtgcttgtgttccgggggcaaccgttggatcaggatcaacagatagctttcgctcgcaattttgggcaattggagggtggattcataaaagttaatcaaagaccaagcagattcaagtacgcggagcttgcggacatctctaatgtctccgttgacgggaaagttgcagatagggaagcccgcgagagcgttggaaacttcgctaataaccaactttggcattctgattcgagctttcagcaggcggcagctaggtatagcatgctgagcgcgtcagtcctgcctccgctgggtggtgataccgaattttgggacatccatgctacgaacctgggtggccgggatgacctgcccagagagctgcagggtcttagagcagagcggtacctccaaaactctcgcttcatcttgggtgacaccgattatagcgagagccaacggaatgccatgcctccagtctcttggcctctcgtgcgcactcatgccgggtccggtcggaagtttctttttatcggtgctcatgcggggcatattgagggacgcccagtcgccgaggggagaatgttgctcgccgaactgcttgagcatgcgacgcaacggaaatttgtgtatcggcaccggtggaaggtgggcgatttggtcatgtgggataacagatgcgttttgcatcgcggtcgggggtacgatattacagctaggcgggagctcaggagagctacaactgtggacgatggcgtggtt

SEQ ID NO: 59 - protein, 294 aa, Artificial Sequence
  Synthetic polypeptide, QYD30, D30
MALSLTPLTPGFVAEASGLDLTRPLDAAAVRTIEQAMDDYAVLVFRNQPLDQDQQIRFAQGFGPLDLGLRKVKGGAHRFDHAELADISNVTVDGSVAGRDHAKIVGNIANQLWHSDSSFQRPRAKYSMLSAVVVPQDGGQTEFADLRAAYDHLPDGMKAAIGDLQAVHYALHSRFLLGDTEYTQAQRDALPAVRWPLVQTDPRSGRKILFVGIHACEVVGMTVAEGRMLLLDLLEHATQREFVYRHHWRVGDLVMWDNTATLHRGRRFDLAQRRELRRATTEEVRLSAEARAAA

SEQ ID NO: 60 - DNA, 882 bp, Artificial Sequence
  Synthetic polynucleotide
atggcattgagcctcacaccgctcactcccggtttcgttgcagaagcttcggggctggaccttaccagaccgctggacgcggcagccgtgcggaccattgagcaggctatggacgactatgctgtcttggtttttcgcaatcaacccttggaccaagaccaacagattaggtttgcccaggggttcggcccattggatcttggtttgagaaaggttaaaggtggtgcacacaggttcgatcacgctgagctcgccgacatttcaaacgtcactgttgatggcagcgtcgcggggagagaccatgcgaagatagttgggaatatagccaaccagctgtggcactccgattcgtcattccaaaggcctcgcgcgaagtactcgatgctctccgcggttgtggtcccgcaagatggtggtcaaactgaatttgcagaccttcgggctgcctacgatcatctgccggatggtatgaaagcggcgattggtgacctgcaagccgttcattacgccctgcactcccggttcctcctcggggatacagaatacacacaagcgcaaagagatgcgctccctgcagtgcgctggcctctcgtccagaccgatcctcgctcaggccgcaaaattctgtttgtcggtatacacgcttgcgaagtcgtcggcatgacagtggcagaggggagaatgctgctgctggatctcttggagcatgctacccaaagggagttcgtttatagacaccattggagagtgggcgatttggttatgtgggataatacggctacgttgcacagaggtcgcaggttcgacttggctcaaagacgggagttgaggcgggcaaccacggaggaagtgaggctctccgctgaagctagggccgcagcc

SEQ ID NO: 61 - protein, 305 aa, Artificial Sequence
  Synthetic polypeptide, QYD31, D31
MKVEHLTEATFGSVVTDIDLETIDDAEWRELYDLWIERALLVFPGAFLSAEGQDAFARRFGDLEFSRAALSNIGKDGKVHHENDDEVVKSLRGNEGWHIDSTYMPVQAKGAVFSAEIVPSSGAATGWADMRSAYEELDAETRERLADLTAYHSLYYSQGRAGNLPDEPNEDGTYNLYGYHDMEVSLRPLIKIHPITERPSLLIGRHAHDIVGMDPAESESLLDDLNDWACQPPRTYHHQWDVGDAVLWDNRRLQHRGTPFDMTEPRRMWHTRIAGDPSSELALNHHPDTVPSDDVERHPTGTWAC

SEQ ID NO: 62 - DNA, 915 bp, Artificial Sequence
  Synthetic polynucleotide
atgaaagttgagcaccttacagaggctactttcggtagcgtcgtgaccgacatcgacctggagacgattgacgacgcggagtggagagagctgtatgatctttggatcgaaagagccctgctcgtttttcccggagcatttctgagcgccgagggacaggacgcgttcgcacggcgctttggcgacctcgaattctcaagagctgccctgagcaatatcggcaaagatggaaaggtccaccatgagaacgatgatgaggtcgtcaagtcgctgcgcggtaacgagggctggcacatcgactcgacctacatgcccgttcaagcgaaaggagctgttttctcggccgaaatagtcccctccagcggggcggccacaggctgggccgacatgagatcagcatacgaggaactggacgcggaaacaagagaacggctggcggaccttaccgcctatcacagcctgtactactcgcaaggccgggcaggtaacctccctgacgaaccgaatgaagatgggacctataacctctatggttaccatgatatggaagtttcattgcgccctttgataaaaatccaccccataacggaaaggccctccctcctgatagggaggcacgcacatgatatagtcggcatggaccccgctgaaagcgaatcgcttctggacgatttgaatgactgggcatgccaacctccgagaacgtatcaccatcaatgggacgttggcgacgcggttctgtgggataacagacgcctccaacacaggggaacgcccttcgatatgactgaaccgagaaggatgtggcataccaggattgccggagatccttcgtctgagcttgcacttaatcaccacccagacactgtgccgtccgatgacgttgaaaggcatccgacgggtacgtgggcctgc

SEQ ID NO: 63 - protein, 294 aa, Artificial Sequence
  Synthetic polypeptide, QYD32, D32
MSQMTFEPLSGPTFGARVHGVKLTAMDDAAFRELHDNWLEYGLLIVPEQFLTREEQIAFARRFGDLEFEIAEIGNMRDDGSLRPDDGSDEMMKINRGNMGWHHDSTYMPVQAKGAVFSAEIVPQEGGATGFADMRAAYDELDDAMKARIENLRAHHSLHHSQGKVGHKTKPVKDGKVNAGGTFGGYGLHDGPVSVRPLVKVHPETGRKNLLVGRHAYDIIGMSSEESEKLLQDLVDFACQPPRIYHHQWAPGDAVIWDNRRLMHQATPWPFDQARRMWHSRIAGDPATEAAMAA

SEQ ID NO: 64 - DNA, 882 bp, Artificial Sequence
  Synthetic polynucleotide
atgagccagatgacttttgagccactctccggtcccacgttcggagcaagagtccatggggtcaagctcaccgctatggatgatgcggcgtttcgcgagcttcacgataactggctggaatatggactcctcatagtgcctgaacagttcctgacgagagaggaacaaatcgcctttgcaagacgctttggcgacctggagtttgagatagcggaaattggcaatatgagagatgacgggtcactgcggccagacgatggttctgacgagatgatgaagatcaataggggaaacatgggttggcaccacgactcaacctatatgccggttcaagcgaaaggcgcggttttttcagctgaaattgtcccccaagaaggaggtgcgactggttttgctgatatgagggctgcatacgatgagcttgatgacgccatgaaagcacgcatagaaaacctccgggctcaccattctcttcatcattcacagggcaaggtcgggcataaaacaaaacctgttaaggatggcaaagtcaacgcggggggcacctttggtggttacggactgcatgatggaccagtctcagtcaggcctttggttaaggttcatccagaaaccgggaggaagaacctcctggtgggcaggcatgcgtatgacataataggtatgtcctccgaagaaagcgagaaattgcttcaagatctcgtcgactttgcatgccagccgccccggatttaccatcaccagtgggccccaggtgatgcagtcatttgggacaaccgcaggttgatgcaccaagctacgccctggccctttgatcaagctagaaggatgtggcattcgcgcatagcgggagatccggctacagaagcagccatggctgcc

SEQ ID NO: 65 - protein, 286 aa, Artificial Sequence
  Synthetic polypeptide, QYD33, D33
MSEHFDVEPLDASFGARITGLKLTEMSDAAFDALYETWLEHGLLLFPDQHMEPEAQVDVARRFGPLEVKLFAISNLKDDGSVRTSDEDDLVKILKGNMGWHHDSTYMPLQAKGAVFSAHVVPSEGGETGFADMRQAWDALDDDMKRRLEGLTALHSLHYSQMKAGFRRKEKDSEYIGYGLDERPPVRRQMVKVHPETGRKSLLVGRHAFGVSGMSEKESEEFVDGLNEFAGSGDWTYHHKWTPGEVVLWDNRCLMHRACPWDMTKPRVMYHSRIAGDPVSEAEVAA

SEQ ID NO: 66 - DNA, 858 bp, Artificial Sequence
  Synthetic polynucleotide
atgagcgagcacttcgacgttgagcccctcgatgcttcatttggtgctagaatcaccgggcttaagctcacagaaatgtccgatgcagcgttcgacgcactgtacgaaacttggttggaacacggactcctccttttcccggatcagcacatggagcccgaagcccaagtggacgtcgcaaggcgcttcggcccactggaagttaaattgttcgcaattagcaacttgaaggatgacggtagcgtgcggacatccgatgaagatgatttggtcaagattctgaaaggaaatatgggctggcatcatgactccacatatatgccacttcaggcgaagggcgcagtcttttcggcccatgtcgtcccctcggaagggggcgagacgggttttgcggatatgcggcaagcatgggacgcccttgatgatgacatgaagaggagacttgaaggtttgacggcccttcactcccttcactattcacagatgaaagcaggatttcggcgcaaggaaaaggactccgagtatatagggtacgggctcgacgaacggcctcccgttcggagacaaatggtgaaggttcacccagagacagggcggaaatctctcctcgttggtcgccacgcattcggcgtttctggaatgtctgagaaagaaagcgaggaatttgttgacggtcttaatgaatttgccggctcgggtgattggacataccatcacaagtggacacctggtgaggtcgtcctttgggacaataggtgccttatgcatcgggcctgcccgtgggatatgaccaagcctagagtgatgtatcattcgcgcatcgcgggcgatccggtgtcggaagccgaagttgctgca

SEQ ID NO: 67 - protein, 284 aa, Artificial Sequence
  Synthetic polypeptide, QYD34, D34
MSMQVIPLDATFGATVTGLKLSELDEAVFAEIYDHWLRYSLLVFPGQNLDNEQQVTFAKRFGALEIDLVELSNVKDDGSLRTAPDDDRIKILKGNMGWHHDSTYMPVQAKGAVFRADTVPSHGGETGWADMCAAYDALGDAMRNRIEGLSAHHSLTYSQGKAGFKHKDKDSEYNGYGLDQDGAPLRPLVKTHAETGRKTLTIGRHAHAVPGLSQADSEALLTELVDFACQPPRIYHHGWQPGDVVVWDNRCLMHQACPWDMAERRVMFHSRLAGDPEHEFAAPN

SEQ ID NO: 68 - DNA, 852 bp, Artificial Sequence
  Synthetic polynucleotide
atgtctatgcaggtcataccgctggacgcaacctttggggcaacagttaccgggcttaaattgagcgaactcgatgaagccgtctttgccgagatctacgatcattggctgcgctattccctcttggtttttccaggacagaacctcgacaacgaacagcaggtcacatttgcaaaacggttcggtgcgttggaaatcgacttggtcgaactttcgaacgtcaaggacgatggctccttgcggacagcacctgatgacgataggataaaaattctgaaagggaatatgggttggcatcacgactctacctatatgccagtgcaagctaagggggctgtttttagagctgatactgtgccatcccacggaggagaaactggctgggcagatatgtgtgccgcatatgacgcactgggggatgccatgcgcaaccgcatcgagggcctctcggcacaccattctctgacctattcccaaggaaaggccggttttaagcataaggacaaggattcggaatacaatgggtatggactcgatcaagacggcgctccgttgcgccctttggttaagactcatgcggaaacgggacggaaaacgcttactattggcagacatgcgcatgcggtgccgggcctctcacaggctgactctgaagcccttcttactgagcttgttgacttcgcttgccaaccgccaagaatataccatcatggatggcaacccggtgatgtcgtggtctgggacaataggtgcctgatgcatcaagcctgcccctgggatatggccgagcggagagttatgtttcattcgcgccttgcgggggatccggaacatgagttcgcggcccctaac

SEQ ID NO: 69 - protein, 296 aa, Artificial Sequence
  Synthetic polypeptide, QYD35, D35
MSARPFTVEPLPATFGARITGLDLAALDDATFAALYDAWLEYALLIFPGAGLSTAEQVTFARRFGDMEFDIAPLSNVRKDGSLRADDGTDDVVKVLRGNMGWHADSTYMPVQAKGAVFSAHVVPEEGGETGWADMRAAYDALDDDLRRRIEGLSAHHSLVHSQARLGHAHGTDSEYSGYGFHDEAPLRPLVKVHPETGRRSLLIGRHAYGIPGLEETASERLLDELVDFACRPPRVYHHRWQVGDVVLWDNRCLLHRACPWDMSRPRVMYHSRIAGDPVTEAGLAGQSEQPSASGS

SEQ ID NO: 70 - DNA, 888 bp, Artificial Sequence
  Synthetic polynucleotide
atgtccgcgagaccgtttacagtcgagccgcttcccgccacattcggtgcaagaataaccggtttggatttggcggccctggacgatgcgactttcgcagctctttacgatgcgtggttggaatacgccttgctcatcttcccaggggccggcctctcaacggcggagcaggtcacttttgcgcgcagatttggtgacatggagttcgacatcgctccactgagcaacgttagaaaggacggctcactgcgggccgacgacggtaccgacgacgtggtcaaagtgctccgggggaacatggggtggcacgcggattctacctatatgccggtccaggcaaaaggagctgtgttctctgcgcatgtggtgccggaagaaggcggtgaaacaggatgggcagatatgcgcgctgcatatgatgccttggatgacgatcttaggagaagaattgaggggctttcagcacaccactccctcgtgcactctcaagcaaggctgggtcacgcccacggcactgactcagagtacagcggctacggctttcacgatgaggcaccacttaggcccctggtgaaagtccatcccgaaacgggaagacggtccttgttgattggtcgccatgcctacggaataccgggtctggaggagactgcgtcggaacgccttcttgatgagctcgtggatttcgcatgtcggccgcctagggtgtaccaccatcgctggcaagttggagacgtggttctctgggacaatagatgtctgctccatagggcctgcccgtgggacatgtctaggccaagagttatgtaccacagcagaattgccggtgatcctgtcaccgaggctgggttggcaggacaatccgagcagccttcggcgtctggctct

SEQ ID NO: 71 - protein, 284 aa, Artificial Sequence
  Synthetic polypeptide, QYD36, D36
MTHTSPFLVEPLDASFGAVVTDLKLTELGAREFKKLYDTWLEYALLIFPSQFLSNDEQIAFAKRFGDLEFDLVSITNVDKSGQAHFDPNEDWVKSIKGNMGWHCDSTYMPVQAKGAVFTAHTVPSSGGETGWADMRSAYDNLDNKIKKKISGLSAYHSLWYSQSKLGHKPKVGAGYGFHELNPPLRSLVKVHPETGRQSLVIGRHAYGIPGMDPKESQKLLEHLVDFACQPPRIYHHTWTTGDAVVWDNRRLLHQARPWDMKEPRVMYHARIAGDPETEFAAHA

SEQ ID NO: 72 - DNA, 852 bp, Artificial Sequence
  Synthetic polynucleotide
atgacacataccagcccgtttcttgttgaaccccttgacgcgtctttcggggccgtggttaccgatctgaagctcacggagttgggagctagagaattcaaaaaattgtatgacacatggttggaatatgcacttttgatcttcccgtcgcagtttttgtcaaacgacgaacagatagcctttgcaaaaaggtttggcgacctggaatttgatctggtgtcaataactaacgtggacaagtccggccaagcacactttgatcccaatgaagactgggtcaaatctatcaaaggtaacatgggttggcattgtgactccacttacatgccagtgcaagcaaagggagctgtgtttactgcccacactgtgccttcttctggaggggagacaggttgggccgatatgagatcagcctatgacaaccttgataacaagattaagaagaaaatctcggggctgtccgcgtaccactcattgtggtacagccaatccaaacttggtcacaagccgaaagtgggagccggatatggttttcacgagctcaatccaccgttgcgctctctggtgaaagttcatccggagacgggaagacaatcattggttatcggtagacacgcatatggaatccctggtatggaccctaaggagtcgcaaaagctcctggaacatttggtggatttcgcttgtcagccccctagaatttaccaccacacttggaccacaggtgatgcggtggtctgggacaatagaagactgcttcaccaagccaggccctgggacatgaaagagccgagggtcatgtaccatgcaaggatcgctggcgatcccgaaactgaatttgccgcacatgca

SEQ ID NO: 73 - protein, 284 aa, Artificial Sequence
  Synthetic polypeptide, QYD37, D37
MTQASPFLVEPLDASFGAVVTDLKLTELGAREFEKLYDTWLEYALLIFPSQFLNNDEQIAFAKRFGDLEFDLASITNVDKSGQAHFDPNEDWVKSIKGNMGWHCDSTYMPVQAKGAVFTAHTVPSSGGETGWADMRSAYDNLDKKIKKKISGLSAYHSLWYSQSKLGHKPKVGAGYGFHELDPPLRPLVKVHPETGRRSLVIGRHAYGIPDMDPKESQKLLENLVDFACQPPRIYHHTWTAGDAVVWDNRRLLHQGRPWDMKEPRIMYHARIAGDPETEFAAHA

SEQ ID NO: 74 - DNA, 852 bp, Artificial Sequence
  Synthetic polynucleotide
atgacccaggcgtcacccttcctcgtggagccgctcgacgcttcgttcggggcagttgtgactgaccttaagctgacggaactgggtgctcgggaatttgaaaagctctatgatacctggcttgagtatgcgctcctcatcttcccaagccaatttttgaataacgatgaacagatcgcgtttgcaaaacgctttggggatctggaatttgaccttgcttcgattaccaatgtggataaatctggtcaagcccatttcgaccctaacgaggattgggtcaaatccattaaaggcaatatgggttggcattgtgattccacatatatgccagttcaagctaagggagccgtgttcactgctcacacagtcccgtcgtcgggcggagaaacaggttgggcggatatgcgctccgcctacgataacctggataagaagatcaaaaagaaaatctctggccttagcgcctatcacagcttgtggtattcccagagcaaactgggccataagccaaaggttggcgcagggtatggcttccatgaactggatcctcccctccgcccgctggtcaaggtgcatccagagacaggaaggcgctctctcgttataggacgccatgcttacggaataccggatatggatccgaaagaaagccagaaactcttggagaacttggtcgactttgcatgtcagcctcctagaatttatcaccacacgtggacagcgggggacgcggtcgtttgggacaacagacggcttctccaccagggtcgcccgtgggacatgaaggaaccacgcataatgtaccacgctagaatagctggcgaccccgaaaccgagtttgcggcgcacgca

SEQ ID NO: 75 - protein, 293 aa, Artificial Sequence
  Synthetic polypeptide, QYD38, D38
MTDTKPFRVEPLNATFGAVVTGLKLAELDEATFSTLYQSWLEYALLVFPDQHLTNDCQIAFARRFGNLEFDISAITNVDKDGKTHHDEKEDWVKSIKGNMGWHTDSTYMPIQAKGAVFTAHEVPGAGGETGWADTQAAYYALPEDIKAKITGLSAYHSLWYSQSKLGHKPAPGTSYGFHNMDPPLRPIVKIHPETGAKCLTIGRHAHNIPGLEPRESDEILQMLVEFTVQESRVYHHQWKPGDAVVWDNRRLMHQARPWDMRERRVMYHSRIAGDEASEFAASDDNAPRYVSA

SEQ ID NO: 76 - DNA, 879 bp, Artificial Sequence
  Synthetic polynucleotide
atgaccgacacgaagccatttagggtggaaccactgaacgcaacattcggtgcagtcgtcacagggcttaaactcgcagaacttgacgaagcaaccttttccacactctaccagtcgtggctcgagtacgctcttttggtctttcccgaccagcatttgaccaacgattgccaaatagcgtttgcacggaggttcgggaacctggagttcgacatatccgcaatcacaaacgtggacaaggacgggaagacgcatcacgacgaaaaagaggactgggtgaaatccattaagggcaatatgggatggcataccgattcaacgtatatgccaattcaggcgaagggagcggttttcactgcacacgaggtgcctggggctggtggtgaaacagggtgggctgatactcaggcagcatactatgcactccctgaagatataaaggctaaaataactggactttcagcctatcactcattgtggtattcgcaatcgaaactcggacataaacctgctccgggaacttcctatgggttccacaatatggaccctccgctccggccaatagtcaaaatacaccctgagacaggagcgaaatgtctcacaattggtagacacgcgcataacatacccggcttggaaccgagagaatcggatgaaatcttgcagatgttggtggaattcacagttcaggagtcacgggtttatcatcaccagtggaaacccggggatgcagttgtttgggataacaggagacttatgcatcaggcacgcccctgggatatgagggagcggcgcgtcatgtatcacagccgcatagcaggtgatgaagcctccgagttcgccgcgtcagatgacaatgcgccgcgctatgtctcggcg

SEQ ID NO: 77 - protein, 289 aa, Artificial Sequence
  Synthetic polypeptide, QYD39, D39
MSFDVQPLDATFGAVVTGLKLAELDDAEFDALYQTWLEYALLVFPGQHLSNEDQVTFAKRFGTMEFDLSAITNVDKDGKVHHDRKEDWVKSIRGNMGWHTDSTYMPVQAKGAVFTAHVVPSIGGETGWADTQAAYEALDPAVQARIADLSAYHSLWYSQSKLDHKPKVGAAYGFHDMDPPLRPLVKVHPETGRKCLTIGRHAHNIPGLTQAESDALLDDLVAFTVQAPRVYHHSWTPGDAVVWDNRRLMHQACPWDMHERRIMYHSRIAGDEASEFAGSDDAAPRYVSA

SEQ ID NO: 78 - DNA, 867 bp, Artificial Sequence
  Synthetic polynucleotide
atgtcatttgatgttcaacctctcgatgcaaccttcggagcggtggttactgggctgaagctcgcagaacttgacgacgcggagtttgacgctttgtaccagacctggctggaatatgcgttgcttgtgtttccgggccagcacttgtcaaacgaggatcaggtgacttttgccaaacggtttggtacgatggagttcgatctctccgctataaccaatgttgataaagatggcaaggtgcatcacgacaggaaagaagattgggtgaagtcgataagggggaatatgggctggcacacagactccacctacatgccggtccaggcaaaaggagcggtttttacagcgcacgtggttcctagcatcggcggtgaaacaggctgggcagacactcaggcagcgtatgaagcactcgaccccgccgtccaagctcggattgcagatctctctgcctatcattctctttggtactctcagtccaagctggaccataagcccaaagtcggtgcggcttatggattccatgatatggacccgccgctgcgccctctcgttaaagtccaccccgaaacggggagaaagtgcttgaccatcggtaggcatgcgcataacataccaggacttacacaagcagaatctgacgccttgctcgacgatcttgttgcgtttactgttcaggcccctcgggtttatcaccattcgtggacacccggagacgctgtcgtttgggataacagaaggcttatgcatcaagcctgcccgtgggatatgcacgagcggcgcatcatgtatcactcgagaattgcaggcgacgaggctagcgagttcgcaggctcagacgacgctgctcctcgctacgtttcagca

SEQ ID NO: 79 - protein, 287 aa, Artificial Sequence
  Synthetic polypeptide, QYD40, D40
MTGTAFDVEPLDASFGAIVTGLELARMDEAVFGKLYNTWLEYALLIFPGQHLSNDEQITFARRFGDLEFDLAPISNVRADGTVRAEHQDDDVINILKGNMGWHCDSTYMPVQAKGAVFTAHVVTSVGGETGWADMRAAYDALDEPTRDRIADLSAYHSLYYSQAKLGHEPKKHSDYSGYGFHDQDPPLRPLVKVHPETGRRSLLIGRHAYGIPGLTEEESTQLLAGLVDFACQAPRIYHHAWSPGDAVVWDNRCLLHQACPWDMSQPRVMYHARIAGDQATEFAAHA

SEQ ID NO: 80 - DNA, 861 bp, Artificial Sequence
  Synthetic polynucleotide
atgacaggcacggcatttgacgttgaaccattggatgcctcttttggcgctattgtcacgggacttgaacttgcacggatggatgaggccgtctttgggaaattgtacaatacgtggttggagtatgcactgctcatctttccggggcaacacttgtcgaatgatgaacagattacgttcgcaagaagatttggagaccttgaattcgacctggcccccataagcaacgttcgggccgatggtacagtgagggcagaacaccaggacgacgacgtcatcaatatcctcaaaggcaacatggggtggcattgcgattccacctacatgccagttcaagcgaagggggccgtctttacagcccatgtggtcacgtcagttggcggagaaaccgggtgggcggatatgcgcgccgcctatgatgctttggatgaaccgacaagggatcgcatcgctgacctctccgcgtaccatagcctttattattcccaagctaaactcggccatgaaccgaaaaagcactcggattacagcggatacggatttcacgatcaagacccgccactcaggccacttgttaaagtccacccagagacgggaagacggagcttgctgatcgggaggcatgcttacggaataccgggtctgacggaagaagagtcaacacaactcctcgctggattggtcgatttcgcatgtcaggccccgcgcatttaccaccatgcctggtcacctggggatgcagttgtctgggataacaggtgcttgttgcatcaggcttgcccatgggatatgtctcagcctagagtcatgtatcacgctaggattgccggcgatcaagccaccgaatttgccgcccacgct

SEQ ID NO: 81 - protein, 293 aa, Artificial Sequence
  Synthetic polypeptide, QYD41, D41
MPAISGFQEEASMNFDVKPIDATFGAYVTGLDLRSLDEKDFADLYNCWLKYSLLVFPGQHLTTEEQVTFAKRFGKMEFDLSPISNVDKDGSVVKSDEKDVVKMLKGNMGWHQDSTYMPVQAKGAVFSAHVVPSKGGETGWADLTAAYEALDDATKEKIKDLKAYHSLHYSQQKMGFRQKDGYGLHGDAPPLRPLVKVHPETGRTCLTIGRHAYGIPGLSDAESETLLEELVNFTVQPPRIYHHHWEAGDSVIWDNRCLLHQATPWDMNEPRVMYHSRIAGDPKSEFAESVSAA

SEQ ID NO: 82 - DNA, 879 bp, Artificial Sequence
  Synthetic polynucleotide
atgcctgcaatttctggatttcaagaagaggcgtcaatgaactttgacgtcaagccgattgacgccacgttcggagcgtatgtcaccggtctggatctcaggtcgcttgacgagaaggactttgcagatctgtataactgttggctgaagtacagcctgttggttttcccaggacagcacctgaccactgaggagcaagtgacattcgcgaaaagattcggaaaaatggagttcgacttgtcgccgatttccaacgttgataaggacgggagcgtcgtgaagtcggatgagaaagatgtggttaaaatgttgaagggcaatatgggctggcatcaggactcaacgtatatgcccgttcaggccaaaggggccgtgttctcggcgcacgttgtgcctagcaagggaggcgagactggttgggccgacctcacagcggcctatgaggctttggacgatgcaacaaaggagaagataaaagatctgaaagcctaccactccttgcactattcccaacagaaaatgggctttcggcagaaagacggttatggtctgcatggtgacgcgccacctcttcgccctcttgtgaaggtgcaccccgagacgggtcggacttgcctgactattggacgccacgcctacggtattcccggcctgagcgatgcagagtcggaaactctcctggaggagcttgttaactttaccgttcaaccgccgagaatatatcatcatcactgggaagcaggcgacagcgtcatctgggataacaggtgtcttctgcatcaagcgacaccgtgggacatgaacgagccacgggtgatgtatcatagccggatagctggagatccgaaatccgagtttgcagaatcagtctccgctgcc

SEQ ID NO: 83 - protein, 289 aa, Artificial Sequence
  Synthetic polypeptide, QYD42, D42
MQITPIADATFGATVTGLRLATLDDAGFARLHAAWLQHALLVFPGQFLTNAEQIGFAKRFGPIERIGGGDIVAISNVKADGTVRQHSPAEWDDMMKVIVGNMAWHADSTYMPVMAQGAVFSAEVVPPTGGRTCFADMRAAYDALDDATRALVHQRSARHSLVYSQSKLGHVQQAGSAYVGYGMDTTATPLRPLVKRHPETGRLSLLIGRHAHAIPGLEEAESERFLEDLVAWACQPPRVVAHHWTAGDVVLWDNRCLLHRAEPWDFTLPRVMWHSRLAGHPETEAAPLV

SEQ ID NO: 84 - DNA, 867 bp, Artificial Sequence
  Synthetic polynucleotide
atgcagattactccgatagcagacgcaacttttggcgctacggttacgggcttgcggcttgcgaccttggacgacgcaggtttcgccaggttgcacgctgcatggctgcagcatgcgttgctcgtcttccctgggcaattcttgacgaatgcagaacagatcgggttcgcgaagcgcttcggccccatcgaaagaattgggggaggagatatagtggcgataagcaatgtgaaggccgatggtactgttcggcagcactccccagcggagtgggacgacatgatgaaagtgattgtgggaaacatggcctggcatgctgactctacatatatgccggttatggctcagggagctgtcttcagcgcagaggtcgttccccccacgggaggtcggacttgtttcgcggacatgagggcagcgtacgatgcgttggatgatgcgactcgggcgttggttcatcaacgcagcgcccggcattccctggtttattctcaatcaaagctcgggcatgtgcaacaagccggctcggcgtatgttgggtacggcatggacactacagcaaccccgctgagacctctcgttaagcggcatccggaaaccggcaggctgtccctcctgatcggtcggcatgcacatgcaattcctggactggaagaagccgaaagcgagcgctttctggaagatttggtcgcctgggcatgtcagccgccgcgggttgtcgctcaccactggaccgcgggagatgttgtcctctgggacaataggtgtttgttgcacagggctgaaccctgggactttacactgccaagagttatgtggcactcacgcctggcaggccacccagaaactgaggccgcaccgctggtc

SEQ ID NO: 85 - protein, 299 aa, Artificial Sequence
  Synthetic polypeptide, QYD43, D43
MRRLHIAPTGATLGATVTGVSLAALDEPTWQAIYRAWLEHALLIFPGQHLSNQQQIGFARRFGAIERIGGGEIVAISNVKADGRVRAHTPAEWDDMMKVIVGNMAWHADSTYMPVMAKGAVFSGELVPTERGETSFADMRAAYDALDQATRALVHQRSAHHSLVYSQSKLGHVPGAQSAYLGYGMDQTTAPLRPLVKVHPETGRQSLLIGRHAHAIPGMDPAESEVFLEGLVEWACQPPRIHTHHWTAGDVVLWDNRCLLHRAMPWDLAQPRVMWHSRLAGDPVAEAALPPVAPAAATH

SEQ ID NO: 86 - DNA, 897 bp, Artificial Sequence
  Synthetic polynucleotide
atgagaagattgcacatagcaccaacgggggccactttgggcgctaccgtgactggggtgtctttggcggcgttggatgaaccgacctggcaggctatatacagggcttggctggaacacgccctccttattttcccaggtcaacacctgtccaatcagcaacagattggtttcgcccggagatttggcgccatcgagaggattggcggcggcgagatcgtggccatcagcaacgttaaggccgatggaagggtgcgggcgcatacacctgccgaatgggacgacatgatgaaagtgatagtcggaaatatggcctggcatgccgatagcacctacatgcccgtcatggcaaagggcgctgttttctcaggcgaactcgtgccgacagaaagaggtgaaacatccttcgccgatatgagggccgcgtacgatgctcttgaccaagctacccgcgccctcgtccatcagaggtcagcccatcattctctggtttacagccagtcaaaactcggacacgttcctggtgctcagtccgcgtacctgggttacggtatggatcagactacagctcctctccggccactcgtcaaggtccacccagagactggaaggcagtccctcctcattgggaggcatgcgcatgcgatcccgggtatggacccggcagaatcagaggtcttccttgaagggctcgtcgaatgggcatgtcaaccaccacggattcatacacatcactggaccgctggagacgtcgtcctttgggacaacagatgccttcttcatcgggcaatgccttgggacctggcacaaccccgggtgatgtggcactcgaggctcgctggtgatcccgtcgcagaagccgcacttccccctgtcgcacctgcagcagccactcat

SEQ ID NO: 87 - protein, 290 aa, Artificial Sequence
  Synthetic polypeptide, QYD44, D44
MARAFDIAPLDATFGAVVTGVKLAEVDDAGWCDLHAAWLEHALLVFPDQHLTRAQQIAFARRFGPLEFEMAAISNVRPDGSLRVESDNDDMMKILKGNMGWHADSTYMPVQAKGAVFSAEVVPGSGGQTGFADMRAAYDALDENLKARVETLQARHSLHYSQSKLGHETKTADGEYSGYGLHDGPVPLRPLVKVHPETGRKSLLIGRHAHAIPGMERAESERLLQQLIDFACQPPRVYHHDWAPGDAVLWDNRCLLHQATPWDMTQKRIMWHSRIAGDPASETALAGAGP

SEQ ID NO: 88 - DNA, 870 bp, Artificial Sequence
  Synthetic polynucleotide
atggcccgggcatttgacatcgcacccctcgatgctacatttggagccgttgttacgggcgttaaattggcagaggttgatgatgcagggtggtgcgatcttcacgcagcttggctcgaacacgctcttctggtgtttcccgaccaacatttgacgcgggctcaacagattgcatttgcccggcggtttggcccccttgaattcgagatggcggccatttcaaatgtgcgccccgatgggagcctcagggtggaaagcgataacgacgatatgatgaaaatcctgaaaggtaacatgggttggcacgctgattccacatacatgcctgtgcaggctaagggggcagtgttttcagctgaggttgttccaggttctggtggccagactggatttgcagacatgcgggcagcttacgacgcactggatgagaatttgaaagcgcgggtcgagacgttgcaagcgagacattccctgcactactctcagagcaaacttggccatgagactaaaacggccgatggtgagtattcggggtatggtctccatgatggtcccgttccgttgaggcctttggtcaaggttcacccggaaaccggccggaagtcgttgcttattgggcgccacgcccatgcaattcccggtatggaacgcgctgagtcggagcggctcttgcaacaattgatagactttgcctgtcagccgccacgggtttaccatcacgactgggctccaggagacgcagtcttgtgggataacagatgccttcttcatcaagctactccatgggatatgactcagaagcgcatcatgtggcactcaagaattgccggagatcctgcttccgagaccgccctcgccggagcaggtccc

SEQ ID NO: 89 - protein, 287 aa, Artificial Sequence
  Synthetic polypeptide, QYD45, D45
MARPFDIAPLDATFGAIVTGVKLAGLDEACWRALHAAWLQYALLVFPNQHLARVEQIAFAKRFGPLEFEMAAISNVRADGSLRVETDNDDMMKILKGNMGWHADSTYMPVQAKGAVFSAEVVPTIGGQTGFADMRAAYDALDEDLKARVETLQAYHSLHYSQSRLGHETKKADGEYSGYGLHDGPVPLRPLVKTHPETGRKSLLIGRHAHAIPGMEPGESERFLQELIDFACQPPRVYRHNWAPGDAVLWDNRCLLHQATPWDMTQKRIMWHSRIAGDAAGETALAH

SEQ ID NO: 90 - DNA, 861 bp, Artificial Sequence
  Synthetic polynucleotide
atggcgaggccattcgatatagctcctttggacgcaacctttggtgccattgtcactggcgttaagttggcgggattggacgaagcatgctggcgggcccttcatgccgcatggcttcagtatgctcttttggttttcccgaatcaacatcttgccagagtggaacaaattgcatttgcgaaaagatttggacctttggaattcgagatggcggccataagcaacgtccgggcagacgggtcactcagagtggagacagataatgatgatatgatgaaaatccttaagggtaatatgggatggcatgctgactctacttacatgcctgtccaggctaagggcgctgtgttttcggcggaggtggttcctacgatcgggggccagactgggtttgctgacatgagggcagcatatgatgcacttgacgaggacctcaaggctagagttgagaccttgcaagcatatcactcgctccactacagccaatcgcgcttgggtcacgaaaccaaaaaggcggacggtgaatactcaggatatggattgcacgatgggccagttccgctgagacccctggtcaagacgcatcctgagactggcagaaaatctcttctgataggacggcatgcccacgctatcccagggatggaaccgggtgagtccgagcgcttcctccaagaattgatagactttgcatgccaaccgccaagggtttatcgccataactgggccccaggagatgcggtgctgtgggataatcggtgcctcctccaccaggctaccccttgggatatgacgcaaaagaggataatgtggcacagccggatagcgggcgatgcggctggtgaaacagcgctggcgcat

SEQ ID NO: 91 - protein, 287 aa, Artificial Sequence
  Synthetic polypeptide, QYD46, D46
MARAFYITPLDATFGAVVTGVKLVDLDDAGWRELHAAWLQYALLVFPGQYLKRDQQIAFARRFGPLEFEMVAISNVRSDGTLRVEKDNDDVMKILKGNMGWHADSTYMPVQAKGAVFSAEVVPTIGGHTGFADMRAAYDALDDVRKARVENLQARHSLHYSQSKLGHETKKSDGEYHGYGLHDGPVPLRPLVKTHPETGRKSLLIGRHAHAIPGMDGPESEAFLQELIDFACQPPRIYHHDWAAGDAVLWDNRCLLHQATPWDMTQKRIMWHSRIAGDPASESALAH

SEQ ID NO: 92 - DNA, 861 bp, Artificial Sequence
  Synthetic polynucleotide
atggcaagagcattttatataacgcctctggatgctacgtttggagctgttgttacaggcgtcaaactcgttgaccttgacgacgccggatggagggaacttcacgcggcgtggctccaatacgcgctgttggtcttcccagggcagtatcttaaacgcgatcaacagattgccttcgcccggagattcggaccccttgaatttgagatggtcgctatttccaacgtgcgctcagacggcacccttagagtggagaaagataatgacgacgttatgaaaatcctgaagggcaacatgggatggcatgcagatagcacctacatgcctgtccaagcaaaaggggccgttttttccgctgaagttgtgcccaccattgggggacacaccggtttcgccgacatgcgggccgcatacgacgcgcttgacgatgttcgcaaagcccgcgtggagaatctgcaagcgagacacagccttcactatagccaatccaagctcggtcacgaaactaaaaagtctgatggcgagtaccacggatatggtctgcacgacggcccggttccactgcggccactggtgaagacccaccccgagacagggagaaaatcacttctcataggcaggcacgcacacgccattcctggtatggatggaccggagagcgaggcgttcctccaagagctcatagactttgcctgccagccgccgcggatatatcaccacgattgggccgccggcgatgccgtcctttgggataacagatgccttctccatcaggctacaccctgggacatgacgcaaaaaagaattatgtggcactccaggattgcgggcgatccggcctcggaaagcgcactggcacac

SEQ ID NO: 93 - protein, 287 aa, Artificial Sequence
  Synthetic polypeptide, QYD47, D47
MATRLEIKPLDATFGAVVTGIKLAEIDEATWRELHATWLKYALLVFPGQFLKRDQQIAFAKHFGPLEFEMAAISNVKSDGTLRIEKDNDDVMKILKGNMGWHADSTYMPVQAKGAVFSAEEVPTVGGQTGWADMRAAYDALDDARRARVETLQAYHSLHYSQSKLGHETRKSDGEYSGYGLHNGPIPLRPLVKTHPETGRKSLLIGRHAHDIPGMDREESQSFLEELVDFACRPPRIYHHDWAAGDAVVWDNRCLLHQATPWDMTQRRIMWHSRIAGDPASESALAA

SEQ ID NO: 94 - DNA, 861 bp, Artificial Sequence
  Synthetic polynucleotide
atggcgacacgcctggagatcaagcctctggatgctacatttggcgcggttgtcaccggaatcaaactcgctgagattgacgaggcaacttggcgcgaactccacgcaacatggttgaaatatgccctcctggtctttccaggtcagttcctgaaacgcgatcaacagatcgccttcgcgaaacatttcgggcctctcgaattcgaaatggctgctatttctaacgtgaagtccgacgggacgcttaggattgagaaagacaatgatgatgttatgaagattttgaaaggtaacatgggctggcatgcagattcaacttacatgccagtccaagcaaagggtgccgtgttttcagcagaggaggtgcctacagtcggcgggcagacaggttgggcggatatgcgggcggcttacgatgcacttgacgatgcaaggagagctcgggtcgaaacattgcaggcctatcattccctgcattattcccagtccaaattgggtcacgagacaagaaagtcagatggcgaatactctggatatggtctccataatggccccataccgcttaggcctctggtcaagacgcatccagagactggacggaaatcacttcttattggcaggcacgcgcatgacatccccggcatggaccgcgaggagtcccagtcatttctggaagaactcgtcgacttcgcatgtaggcctcctcggatctaccatcacgattgggccgctggggatgcagtggtgtgggacaaccgctgtctgttgcatcaagcaactccttgggatatgactcaaaggcggatcatgtggcactctaggattgccggtgaccccgcatcggaaagcgccctggctgcg

SEQ ID NO: 95 - protein, 287 aa, Artificial Sequence
  Synthetic polypeptide, QYD48, D48
MTDRLEIKPLDATFGAVVTGIRLAEIDEPTWRELHATWLKYALLVFPGQHLQRDQQIAFAKRFGPLEFEMAAISNVKSDGTLRIEEDNDDMMKILKGNMGWHADSTYMPVQAKGAVFSAEEVPTIGGQTGWADMRAAYDALDEARKAQIESLQAYHSLHYSQSQLGHQTGKSDGEYSGYGLHDGPVPLRPLVKTHPETGRKSLLIGRHAHNIPGMDRDEARRLLEGLIDFACQPPRIYHHDWAAGDAVVWDNRCLLHQATPWDMTQRRIMWHSRIAGDPASESALAA

SEQ ID NO: 96 - DNA, 861 bp, Artificial Sequence
  Synthetic polynucleotide
atgacggaccgcctggaaatcaaacccctggatgccacattcggcgctgtcgttaccggcatacggttggctgaaatagatgagccgacgtggcgcgagctgcacgcgacctggctgaagtacgcactgctggttttccccggccagcacctgcagcgggatcagcagattgcatttgcgaagagattcggcccgctggaatttgaaatggcggccatatcgaatgttaagtcagatgggacgctccgcatagaggaagataacgacgacatgatgaaaatattgaaggggaacatgggttggcatgcggactcaacatatatgcctgtgcaagcaaagggggccgtgttttctgctgaagaagtccctactataggcgggcagaccggttgggcggacatgagggctgcatacgacgctttggatgaggcacgcaaggcacagattgaatcacttcaggcttaccattcgttgcactattctcaatcacagcttggtcaccagaccggtaagtctgacggcgaatattccggatacggactgcatgacggccctgttcctttgaggccccttgttaaaactcatccggagacggggagaaaatcgctcctgatagggcgccacgcacacaatatccctggtatggatagggatgaggctcggcgcctgcttgaagggttgatagactttgcttgtcagccaccgcgcatttaccaccatgattgggccgcaggagatgccgttgtgtgggataacaggtgtctgctgcatcaagccacgccatgggacatgactcaacggcgcattatgtggcattcccggatcgccggggaccccgcgagcgaatcagctcttgccgca

SEQ ID NO: 97 - protein, 297 aa, Artificial Sequence
  Synthetic polypeptide, QYD49, D49
MARTFDIEPLAGTTFGAVVRGMKVAILDDAGWRDLYAAWLKYALLVFPGQHLKRDEQIAFAKRFGALEFEMAALSNVKSDGTLRQEKDNDDMMKILKGNMGWHADSTYMPVQAKGAVFSAEIVPGAGGQTGFADMRAAYDALDAGRKAKVERLAAHHSLHYSQSKLGHQTKKQDGEYSGYGLHDGPVPLRPLVKTHPETGRKSLLIGRHAHNIPGMDKAESERLLQELVDFACQPPRVYHHDWTAGDAVVWDNRCLLHQATPWDMTQPRVMWHSRIAGDPRSEAALGVTAVSAAASG

SEQ ID NO: 98 - DNA, 891 bp, Artificial Sequence
  Synthetic polynucleotide
atggccagaaccttcgacattgaaccgctcgctggaacgacgtttggtgcagtggttaggggtatgaaagtcgccatcttggatgatgctggctggcgggatctttacgcagcttggcttaaatatgcacttttggtgtttcccggccaacatttgaagcgggatgagcagattgccttcgctaagaggtttggcgcgctggaatttgaaatggccgccctctctaatgttaagtctgatggtaccctcaggcaagagaaggacaacgacgacatgatgaaaatacttaaagggaacatgggctggcatgctgattcaacatatatgcctgtccaggcaaagggtgcggttttctcggccgagatcgtcccaggtgcgggtggccagacaggcttcgcagatatgcgggcggcttacgacgcgctcgatgccggaagaaaggcgaaggtcgaacgcctcgcggcgcaccattctctgcactactcacagagcaaactggggcatcagacaaagaagcaagatggtgaatacagcggttatggactgcatgatggccccgttccacttagaccgttggtcaaaacacatcctgagactggtcggaagtcccttctcataggtcggcatgcgcataacatacccgggatggataaagccgagagcgaacgccttcttcaggagttggtggacttcgcatgtcaacctcccagggtgtaccaccacgactggaccgcaggagacgcagtcgtttgggacaaccgctgcttgctgcaccaagcgacgccctgggatatgacacaacctcgggttatgtggcactcgagaatagccggcgatcctaggtctgaggcggccttgggtgtgactgccgtctctgctgccgcctctgga

SEQ ID NO: 99 - protein, 302 aa, Artificial Sequence
  Synthetic polypeptide, QYD50, D50
MEEPDMARTLGIEPLAGTTFGAVVKGVKVTALDDATWRELHAAWLQYALLIFPDQHLKRAEQIAFARRFGPLEFEMAAISNVKADGSLRIEADNDDVMKILKGNMGWHCDSTYMPVQAKGAVFSAEVVPSIGGRTGFADMRAAWDALDDALKARIETLSAYHSLHHSQSKLGHQTNKRDGEYSGYGLHDGPVPLRQLVKVHPETGRKSLLIGRHAHNIPGMDKADSERLLQELVDFACQPPRIYHHDWAAGDAVVWDNRCLLHQATPWDMTQPRVMWHSRIAGDPRSEAALGGSAVSAAASG

SEQ ID NO: 100 - DNA, 906 bp, Artificial Sequence
  Synthetic polynucleotide
atggaggagccggacatggctcggacgctcggaatagaaccccttgccggaacaacatttggagcagttgtcaaaggcgttaaagtgactgcgttggacgatgcgacatggagagaactgcatgccgcctggctccaatatgcgctcttgatcttcccggaccagcacctcaaaagggcggagcaaattgcatttgccagaagattcggaccattggaatttgagatggcagcaatcagcaacgtcaaggccgatggctcattgcgcatcgaggccgataatgatgatgttatgaagattttgaaaggcaacatggggtggcactgtgatagcacttatatgcctgttcaggccaagggtgcggttttttccgctgaggttgtcccttctatcggtgggagaacgggattcgcagatatgagggctgcctgggacgcacttgatgatgccctcaaagcccggatcgaaaccctgtcggcttaccatagcctccatcattcccaatctaagcttggtcatcaaactaataaaagagatggagagtactcaggttacgggcttcatgatggtcctgtgcctctgaggcagctcgttaaggtccatcctgagactggtagaaagtctctgctcatcggcaggcacgcccacaacattccgggcatggacaaggcagactctgaacgccttctccaggagcttgtcgactttgcatgccaaccaccaaggatataccatcatgattgggccgcaggcgatgcggtggtctgggataaccgctgcctgcttcatcaagcaacaccatgggacatgacgcaacccagagttatgtggcattctcggattgcaggggatccacggtcagaagcagctttggggggatcagcggttagcgcagcggctagcggc

SEQ ID NO: 101 - protein, 288 aa, Artificial Sequence
  Synthetic polypeptide, QYD51, D51
MARTLDIEPIDATFGAVVRGIKVAEVDDETFRALYDAWLEYALLIFPDQHLKRDQQIAFAKRFGPLEFEMTAISNVRSDGTLRLEKDNDDVMKVLKGNMGWHHDSTYMPVQAKGAVFSAEEVPTIGGRTGFADMRAAYDALDPAMKARIEGLNAYHSLHYSQGRVGHQTKKSDGEYSGYGLHDGPVPLRPLVKTHPETGRKSLLAGRHAHNIPGLAQEESDRLLRELIDFACQPPRIYHHDWTPGEAVVWDNRCLMHQATPWDMTQRRIMWHSRIAGDPASESARPQP

SEQ ID NO: 102 - DNA, 864 bp, Artificial Sequence
  Synthetic polynucleotide
atggccaggaccctggacatagagcccatagacgccacttttggcgcagtggttcggggtataaaagtggccgaggtcgatgacgagacttttcgggcactttacgatgcgtggctcgagtacgctctcctcatcttcccagatcaacacctcaaaagagaccagcaaatagcctttgctaagagattcggccctctcgagtttgaaatgactgcaattagcaacgtgaggtcagacggaaccttgaggctcgagaaagataatgacgacgttatgaaggtcctgaagggtaacatgggttggcaccacgattctacgtatatgcctgtccaagccaagggcgctgtgttttcagcagaagaagtccccacgatagggggtagaaccggcttcgcagacatgagagctgcttatgatgcactcgaccctgcaatgaaagccaggatcgaaggtttgaatgcatatcactcattgcattactcgcaaggtcgggtgggacaccaaaccaaaaaatccgacggagagtattcaggatatggcctccacgatgggccagtcccactccgccccttggttaaaactcatcccgagaccggtcggaagagcctgctggccgggagacatgcgcacaatatacctggtttggcgcaggaggagtcagatagactcctcagggaactcattgattttgcctgtcagccacctagaatctatcaccacgattggacaccgggcgaggccgtggtttgggataacagatgcctgatgcaccaagccactccgtgggatatgacccaacggcggataatgtggcactccaggatcgcaggagacccagcatcagagtcagcgagacctcaaccg

SEQ ID NO: 103 - protein, 287 aa, Artificial Sequence
  Synthetic polypeptide, QYD52, D52
MTRTLDIEPIDATFGAVVRGIKVAEVDDETFRALYDAWLQYALLIFPDQHLQRDQQIAFAKRFGPLEFEMTAISNVRSDGTLRLEKDNDDVMKILKGNMGWHHDSTYMPVQAKGAVFSAEEVPTIGGRTGFADMRAAYDALDPAMKARIEGLNAYHSLHYSQGKVGHQTRKADGEYSGYGLHDGPVPLRPLVKTHPETGRKSLLAGRHAHNIPGLSQEASDSLLRELIDFACQPPRIYHHDWTPGDAVVWDNRCLMHQATPWDMTQRRIMWHSRIAGDPASESSLAA

SEQ ID NO: 104 - DNA, 861 bp, Artificial Sequence
  Synthetic polynucleotide
atgactagaactttggacatagagcccatcgatgcaaccttcggcgcggttgtgcggggtatcaaggtggcagaagtcgacgacgaaacttttagggctctctatgatgcttggctgcaatatgccttgttgatcttccctgaccaacacctgcaacgcgaccaacaaattgcatttgcgaaaagattcggaccattggagtttgaaatgacagcaatatcgaatgtgaggagcgatggtactctccgcttggagaaagataatgatgatgtcatgaagatactgaaaggtaatatggggtggcaccacgactccacgtatatgccagtccaagccaagggagcagtgttttcagctgaagaagtcccgactatcggcggtcgcacaggatttgccgacatgcgggcggcatacgatgcactggacccagcgatgaaggcgaggatagaagggttgaacgcataccattccttgcactattcacaaggtaaggttggtcaccaaacgcgcaaggctgatggtgagtatagcggatacggtcttcacgacggcccagttcctctgaggccgctggtgaaaactcacccagaaacaggtcggaagtcgttgcttgctggaaggcatgcgcataacattccaggtctgagccaggaagcatcggattcgcttctcagggagttgatcgatttcgcctgccagcctcccagaatatatcaccatgattggacaccaggtgatgcagtggtctgggacaataggtgtttgatgcaccaggcgactccgtgggacatgacgcaaaggcgcataatgtggcatagccgcatcgcaggagaccccgcgtccgaatcatcattggcggcg

SEQ ID NO: 105 - protein, 288 aa, Artificial Sequence
  Synthetic polypeptide, QYD53, D53
MASTLSLQPLEGITFGAVATGFRIAELDEPTWQALHDAWLDHALLIFPGQFLTRDEQIAFARRFGPLEFEMAAISNVKPDGTLRLEKDNDDMMKVLKGNMGWHADSTYMPVQAKGAVFSAEVVPTIGGETGWADMRAAYEALDAATREKIDGLSAFHSLHYSQSKLGHQTQKSDGQYNGYGLHDGPVPLRPLVKTHPETGRKSLLIGRHAHNIPGMDKAESERFLDGLIAFACQPPRVYHHHWAPGDVVVWDNRCLLHQATPWDMTQPRVMWHSRIAGDPASEAGLAA

SEQ ID NO: 106 - DNA, 864 bp, Artificial Sequence
  Synthetic polynucleotide
atggctagcactctttctctccaacctctggaaggcattaccttcggtgccgtcgccacaggctttagaatcgctgaattggacgagcccacatggcaggctttgcatgatgcttggctcgatcatgcgcttcttattttccctggtcaatttctcacacgggacgaacaaatcgcatttgctagaagattcgggcccttggagttcgagatggcggcaatatccaatgtcaagcccgatgggaccttgcgcttggaaaaggataacgacgacatgatgaaagttctgaaaggaaacatgggatggcacgccgacagcacttacatgcccgtccaagctaaaggggcggtgttttctgcagaagtggtcccaacgataggaggagagacaggatgggctgatatgagggctgcttacgaagcactggatgcagctacgcgcgaaaagatcgatggcctttcagcgttccatagcttgcactactcacaatcaaaattgggccatcaaacgcaaaaatctgatggacagtacaatggttatggtctgcatgatggccccgtgcctctgcggccactcgttaaaacccacccggagaccggacgcaagtcgctgcttattggtcggcatgcccacaatatacctggtatggacaaggctgagtcagagcggtttctggacggtctcattgctttcgcctgtcaacccccaagggtctaccatcatcactgggcccccggtgatgttgtcgtttgggacaatagatgcctccttcaccaggccactccatgggacatgacccaaccgagggtgatgtggcattcccgcattgcaggtgacccagcttcagaggctgggctggcggcc

SEQ ID NO: 107 - protein, 285 aa, Artificial Sequence
  Synthetic polypeptide, QYD54, D54
MARMFDIAPLDATFGAVVTGVKVTRIDDATWRDLHAAWLEYALLIFPGQFLSRAEQIAFAKRFGPLEFEMAPISNVKADGTIRIEADNDDVVKVLKGNMGWHADSTYMPVQAKGAVFSAEVVPGEGGRTGWADMRAAYDALDAARRVRVESLTAYHSLYYSQAKLGHQPKRGSDYSGYGFHDGPVPRRPLVKVHPETGRKSLLIGRHAHNIPGMDVAESTQFLESLVDFACQPPRIYHHQWTAGDAVVWDNRCLLHRATPWDMTKPRVMWHSRIAGDPVSEASLG

SEQ ID NO: 108 - DNA, 855 bp, Artificial Sequence
  Synthetic polynucleotide
atggcaaggatgttcgatatagcaccacttgatgcgacttttggtgccgtcgtgaccggagttaaagttaccagaatagacgatgctacctggagagacctccatgcggcatggctcgaatatgccctccttatattccctggacaatttctgtctcgcgcagagcaaatcgcctttgcgaagcgcttcgggccactggagttcgagatggcgccaataagcaacgttaaggcagacggtacaattagaattgaagcagacaatgacgatgtcgttaaggttctcaagggaaacatgggatggcatgctgattctacttatatgcccgtccaagccaaaggcgctgttttttcagcggaggttgtcccgggtgaaggtgggagaacaggctgggccgatatgcgcgccgcatacgacgcgctggacgccgctagaagggttagggtcgaatcactgactgcatatcattctttgtattatagccaagccaaacttggtcatcaacccaagagggggtcagactactcaggctatggatttcatgacgggccagtgcctcgccggccactcgttaaggtgcacccggagaccggtagaaagtcactcctcatcgggcgccacgctcataacatacctggaatggacgtggcggaaagcacacagtttctggagtctctggtggattttgcctgtcaaccccccagaatctatcaccatcaatggactgcaggagatgcagtggtttgggacaataggtgcctgctgcatagggccacgccatgggatatgactaaaccgagggtcatgtggcactcgagaatcgcgggtgacccggtctcagaggcctcattgggc

SEQ ID NO: 109 - protein, 285 aa, Artificial Sequence
  Synthetic polypeptide, QYD55, D55
MARPFDIEPLDATFGAVVRGVKVAALDDVTWRALHDAWLQYALLIFPGQHLSREAQIAFAKRFGPLEFEMAPISNVRADGSVRAEAENDDVIKVLKGNMGWHCDSTYMPVQAKGAVFSAEVVPGAGGQTGWADMRAAYDALDGDLRARIENLSAYHSLYYSQAKLDHRPSKSSAYSGYGFHDGPVPLRPLVKVHPETGRKSLLIGRHAHNIPGMDADESTRLLQSLVDFACRPPRIYHHHWTAGDTVVWDNRCLLHQATPWDMTQPRVMWHSRIAGDPASEAALA

SEQ ID NO: 110 - DNA, 855 bp, Artificial Sequence
  Synthetic polynucleotide
atggcgcggccattcgatatcgagccacttgacgccacatttggcgcagttgttagaggggttaaagtcgccgctctggatgacgttacgtggagggcactccacgacgcatggttgcaatatgccttgctgattttccctggacagcacctgtctagagaagcgcaaatagcttttgcgaagaggttcggcccactcgagtttgaaatggccccgatctccaatgttagagcggatgggagcgtcagggccgaggcagagaacgacgatgtcattaaggtcctcaaaggtaatatgggctggcactgcgactctacctacatgcctgtccaagcgaagggcgcagttttttccgcagaggtggtccccggagccggtggccaaacaggatgggcagatatgcgcgccgcctatgatgcactcgacggggatttgagggcgagaattgaaaatctttccgcgtaccattctctttactattcccaagccaagctcgatcatcggccttcaaaaagcagcgcatactcgggctacggatttcacgacggaccggttcccttgcgcccgcttgttaaagtgcatcctgaaaccgggcgcaagagccttctgatcggacgccacgcccacaacattccaggaatggacgcagatgaaagcactaggctcctccagtcgctggtggacttcgcgtgtagacctcctcggatataccatcaccactggactgccggcgacacggttgtttgggataatcgctgccttttgcaccaggccaccccttgggatatgacacaacctcgggttatgtggcattcgcgcatagcgggggacccagcatctgaggcggcactggcg

SEQ ID NO: 111 - protein, 285 aa, Artificial Sequence
  Synthetic polypeptide, QYD56, D56
MARTFDVQPLDATFGAVISGVKLAALDDETWRELYATWLQYALLVFPGQHLSRDQQIAFARRFGPLEFEIIALSNVKSDGTIRSEVENDDVIKVLKGNMGWHADSTYMPVQAKGAVFTAEVVPSVGGQTGWADMRAAYDALDGATRQKVEQLSAYHSLHYSQSKLGHQHKPGSAYSGYGFHDGPVPLRPLVKTHPETGRKSLLIGRHAHNIPGMDKDESARFLEDLVASACQPPRIYHHTWAAGDAVVWDNRCLLHQATPWDMTQPRVMWHSRIAGDPLSEAALG

SEQ ID NO: 112 - DNA, 855 bp, Artificial Sequence
  Synthetic polynucleotide
atggcccgcacgtttgatgtgcagccccttgatgcgacctttggcgcagtcattagcggagttaaactggcggctcttgatgatgaaacgtggagagagttgtatgcgacgtggctgcagtatgcattgctcgtcttcccaggccaacacctttcgcgcgatcagcaaatagcatttgccaggaggtttggcccacttgagttcgagattatcgcactctcgaacgttaaatcggatggtaccattaggagcgaagtggaaaacgatgatgtcattaaggttctcaaagggaatatgggatggcacgcagactccacatacatgcctgtccaggccaagggagcagttttcacggcggaggtggttcccagcgtgggaggtcaaactggttgggctgatatgagagcagcttatgacgcattggacggtgcgactcgccagaaggttgaacaactgtctgcttatcattctcttcactacagccaatcgaagttggggcaccagcacaagccgggctccgcgtactcagggtacggttttcacgacggtcctgttcccctgaggccgttggtcaagactcatcccgaaacggggcgcaagagcctcttgataggtcgccatgcccacaacataccgggaatggataaagacgaatccgcgaggtttttggaggatctggttgcgtcggcgtgccaacctccacgcatctaccatcacacttgggccgcaggcgacgccgtggtttgggataatcgctgcctgttgcatcaggctacgccatgggacatgacccaacccagggtcatgtggcactcacgcattgctggcgaccccctgtctgaagctgctctgggt

SEQ ID NO: 113 - protein, 286 aa, Artificial Sequence
  Synthetic polypeptide, QYD57, D57
MPQHFDVAPVDATFGAVVTNLKLANLDEPAFRELYATWLEHALLIFPGQFLTREQQIAFARRFGALEFEMAAISNVKSDGTLRREADNDDVVKILKGNMGWHADSTYMPLQAKGAVFSAKVVPSARGETGWADMRAAYDALDAAVRDRVERLSAYHSLHYSQAKAGFQHKDKKSEYSGYGLHDGPVPLRPLVKTHPETGRKSLLIGRHAHNIPGMEPEESERLLDGLVEFACQAPRTYHHSWKPGEAVIWDNRCLLHRAMPWDMTEPRIMWHSRIAGDPASEAAIN

SEQ ID NO: 114 - DNA, 858 bp, Artificial Sequence
  Synthetic polynucleotide
atgcctcaacacttcgatgtggctcctgtggatgctacatttggtgctgttgtcactaatcttaagttggcgaatctggacgaacctgcctttagggaactgtatgcgacatggttggagcacgctttgttgatctttccaggacaattcctcacacgcgagcagcaaatagcttttgcccgcagatttggagctcttgaatttgaaatggcagcaattagcaacgtgaaatccgacggaactctcagacgggaagcagataatgatgacgtggtcaagatattgaaaggaaacatggggtggcatgcagacagcacgtacatgcccttgcaggcaaagggagcggttttttccgcaaaagttgttccctcagcacggggtgaaacgggatgggcggatatgcgggcagcgtatgacgcgcttgatgcggccgtgcgcgacagggtcgaaagactttccgcttatcactccctccactattcacaggctaaagctggtttccagcacaaagataagaaatctgagtatagcggttacggtctccatgacggtccggtcccactgaggccattggtgaagacgcatccagagacagggagaaagtcgttgcttattggtcggcacgcgcataacattccgggaatggagcctgaggagtccgagagactgttggatgggcttgtcgaatttgcgtgtcaggctccgcgcacttatcaccactcgtggaaaccaggggaggccgtcatatgggacaaccgctgccttctccatagagcaatgccttgggatatgacggagccgcggattatgtggcactcgagaatcgccggggatcccgcgtctgaggctgccattaac

SEQ ID NO: 115 - protein, 297 aa, Artificial Sequence; identical to SEQ ID NO: 97
  Synthetic polypeptide, QYD58, D58

SEQ ID NO: 116 - DNA, 891 bp, Artificial Sequence
  Synthetic polynucleotide
atggcccgcacgtttgacatcgagccccttgccggcaccaccttcggtgcagtggttcgggggatgaaggtcgccatacttgacgatgctggttggcgggacctttacgcggcatggcttaaatatgcgttgttggtgtttccaggccagcatctgaaacgggatgagcaaatagcgttcgctaaacgctttggcgcgcttgaattcgaaatggccgcgctctcgaatgttaaatctgatggaacgctgagacaagaaaaggacaacgatgatatgatgaaaatcttgaaggggaatatgggttggcatgcggattcgacttacatgccggtccaagcgaagggcgccgttttttccgctgagattgtgcctggagcaggtggacagacaggtttcgcagatatgcgggctgcatatgatgcccttgacgcggggagaaaggcaaaggttgagcggctggccgcgcaccattcgcttcactactctcagtcgaaactgggccaccagactaaaaaacaagacggtgaatactcgggttatggtcttcatgatggtccggtgccccttaggccccttgtgaaaactcaccccgagacagggcggaaatcgttgctgatcggaagacatgcacacaacatccccgggatggataaggccgaatcggaaagactgttgcaggaattggttgactttgcatgtcagccgccgagagtttatcaccacgactggacagccggtgacgcggtcgtttgggacaaccgctgcctgttgcatcaggcaactccttgggatatgacgcagcctcgcgtcatgtggcattcaaggatagctggagacccgagaagcgaagcagctttgggtgtcaccgccgtgtccgcagctgcatctgga

SEQ ID NO: 117 - protein, 284 aa, Artificial Sequence
  Synthetic polypeptide, QYD59, D59
MQTFDIHPFPGSVGAEIIGLDLSRPVNDQDFARIHRAHLDYHVIVFRDQRITPEQQIAFSRRFGVLQIHVLKQFLLANHPEILIVSNIIENGQSIGLGDAGKFWHSDLSYKELPSLGSMLHAQELPSEGGDTLFADMHKAWEHLPGHLRNAVEGRSAAHSYTARYSETKFEGNWRPTLTPEQLAQVAEVVHPVVRTHPENGRKALFVSEGFTTRIVGLPDDESRDLLAQLYAHSVLPENIYRHQWQPHDLVFWDNRSLIHLAAGCPSHLRRKLYRTTIQGDAPF

SEQ ID NO: 118 - DNA, 855 bp, Artificial Sequence
  Synthetic polynucleotide
atgcaaaccttcgacatccatccgtttcctggctcagtcggagccgaaatcatcggcttggacttgtccaggccggtcaatgatcaggacttcgcgagaatacatcgggcccatttggactatcacgtcatcgtgttcagagatcagcgcattacgccagaacaacaaattgcattttctcgcagatttggtgtccttcaaattcacgtccttaagcaatttctgctggcgaaccatcccgaaatccttatcgtttcgaatataatagagaacggccaatccatcggattgggagatgctggtaagttctggcatagcgatttgtcgtataaggagttgccgtcgttggggtctatgcttcatgcccaggaactcccaagcgagggtggcgacactcttttcgccgacatgcataaggcgtgggagcatctgccgggacacttgcgcaacgctgtggaaggccggtctgccgctcactcttatactgccagatattctgaaactaaatttgaggggaactggcgccctaccctcactccagagcaactggcgcaggttgcagaggtcgtccacccggtcgttcgcactcatccggagaatggccggaaagctctgttcgtttccgaaggattcactacaagaattgtcgggcttccagatgatgagtcccgcgacttgttggctcagttgtatgcccactcggtgcttcccgagaacatctaccggcatcaatggcaaccgcatgacttggtgttctgggacaacagatctcttattcacctcgccgcaggttgcccatctcatcttagacggaagctctaccggacaacgattcagggagatgcgccattctaa

SEQ ID NO: 119 - protein, 287 aa, Artificial Sequence; identical to SEQ ID NO: 5
  Synthetic polypeptide, QYD60, D60

SEQ ID NO: 120 - DNA, 864 bp, Artificial Sequence
  Synthetic polynucleotide
atgtctcccgcgttcgacatcgctccattggacgctacttttggcgctgtcgtgactggcgttaagctcgcagaccttgatgacgcggggtggttggaccttcaggctgcatggttggagtacgccctccttgtcttccccgaccaacatctgactcgggaacagcaaatcgctttcgcaaggagattcggcccacttgagttcgagatggctgccatctctaacgtcagacccgacggatcgcttcgcgtcgagtccgacaacgacgatatgatgaagatcttgaagggaaacatgggatggcatgcggactccacatacatgcctgttcaagcgaagggggcagtcttttcagcagaggtggtgcctagcgtggggggacagactggatttgctgatatgagggcggcctacgacgcgttggacgaagatctgaaagctagagtggagacgctgcaggctcggcattcattgcactattcccagtctaaactgggccatcagaccaaagcggctgacggtgaatactctgggtacggcttgcatgatgggccagtccccttgcggccccttgtgaagatacaccccgaaactgggaggaagtctttgcttataggaagacatgcccacgccattcccggattggagccggcggagtcagaaagacttctccagcagctcattgactttgcatgtcaaccgccgcgcatataccaccacgattgggcaccgggtgatgctgtgctgtgggacaaccgctgtcttctgcatcaggctacaccctgggacatgactcagaaacggataatgtggcattcgagaatcgctggcgaccctgcctctgagacggcgcttgctcattaa

SEQ ID NO: 121 - protein, 287 aa, Artificial Sequence
  Synthetic polypeptide, QYD61, D61
MSVVANPLHPLFAAGVEDIDLREALGSTEVREIERLMDEKSVLVFRGQPLSQDQQIAFARNFGPLEGGFIKVNQRPSRFKYAELADISNVSLDGKVAQRDAREVVGNFANQLWHSDSSFQQPAARYSMLSAVVVPPSGGDTEFCDMRAAYDALPRDLQSELEGLRAEHYALNSRFLLGDTDYSEAQRNAMPPVNWPLVRTHAGSGRKFLFIGAHASHVEGLPVAEGRMLLAELLEHATQREFVYRHRWNVGDLVMWDNRCVLHRGRRYDISARRELRRATTLDDAVV

SEQ ID NO: 122 - DNA, 864 bp, Artificial Sequence
  Synthetic polynucleotide
atgtctgtcgtggctaatcccctccatcctctttttgctgccggggtggaggacatagatcttcgggaagccctcggctcgaccgaagttcgggaaatagaaaggcttatggacgagaagtcagttcttgtttttagaggccaaccactgagccaagatcagcaaatcgcatttgctcgcaatttcggtcccctcgaaggaggatttataaaagttaatcaaagaccctccagattcaagtacgcagaacttgcggatatatctaacgtgtcacttgatggaaaagtcgcgcaacgcgatgcaagggaagtcgtgggtaatttcgccaatcaactgtggcacagcgattcatccttccagcagccagcagcaaggtactccatgctgtctgcggtggtcgtgccaccgtcgggcggtgataccgaattctgcgacatgagggccgcgtatgatgcccttccgagagacctgcagtctgaactcgaaggccttcgcgctgaacactacgcgttgaattctcgcttcctcctgggtgatactgactactcagaggcacagcggaacgcgatgcctcctgtcaattggccgcttgtccggactcatgccggctccggcaggaaattcttgtttatcggtgctcatgcgtctcacgttgaagggctgccagttgccgagggcagaatgttgttggctgaattgttggaacacgcaactcagcgggagttcgtctatcggcacaggtggaatgtgggcgatcttgtcatgtgggacaatcggtgcgtgctgcacagaggacggcggtatgacatctctgctcggcgcgaattgaggagagctaccacactggatgatgcagttgtttaa

SEQ ID NO: 123 - protein, 295 aa, Artificial Sequence
  Synthetic polypeptide, QYD62, D62
MTIAIRQLHKHFVGEVSGLDLRKPLTADEARDIEAAMDKYAVLVFHDQDITDEQQMAFALNFGQREDARGGTVTKAKDYRLQSGLNDVSNLGKDGKPLAKDSRTHLFNLGNCLWHSDSSFRPIPAKFSLLSARVVNPKGGNTEFADMRAAYDALDDETKTEIEDLVCEHSLMYSRGSLGFTEYTDEEKEMFKPVLQRLVRTHPVHRRKSLYLSSHAGKVVGMSVPEGRLLLRDLNEHATQPEFVYVHKWKLHDLVMWDNRQTMHRVRRYDQSQPRDMRRATVAGTEPTVQQQAAE

SEQ ID NO: 124 - DNA, 888 bp, Artificial Sequence
  Synthetic polynucleotide
atgactattgccataagacaactgcataaacacttcgtgggcgaggtctcgggattggacttgcggaaacctctgactgctgacgaggcacgggatattgaggccgcaatggacaagtatgcggtcctcgtgttccatgaccaggacataacagacgagcagcagatggcatttgccctcaactttggtcaacgcgaagatgcaagggggggaactgtcaccaaggcgaaggattaccgcctccagtcgggtttgaacgatgtgtcgaacctgggcaaggatgggaaacctttggccaaagattctaggacccacctctttaatctgggtaattgcctgtggcattcggattcctcatttcgccccatacctgccaaattctcattgctctcagcgcgggttgtcaatccaaaagggggtaatacagagttcgctgatatgcgggcagcttatgatgccctggacgacgaaacgaaaactgagatagaagatctggtctgtgagcactcccttatgtattccagagggtctcttgggttcactgaatatacggatgaagagaaggaaatgttcaaacctgttctccaaagactcgtgaggacacatcccgttcatcggagaaagtccttgtatctgtccagccatgctggcaaagtggtggggatgtcggttcccgaaggcagacttctgcttagggacctgaatgaacacgcaacgcaaccggaattcgtttatgtgcataaatggaaacttcacgacctggttatgtgggataaccgccaaaccatgcacagggtccggagatatgaccagtctcagccgagggacatgaggagggcgactgtcgcaggaacggagcctacggtccaacagcaggctgccgagtaa

SEQ ID NO: 125 - protein, 293 aa, Artificial Sequence
  Synthetic polypeptide, QYD63, D63
MSSLELTPLHPLFAAEARGIDIRQPLSPSEVKQINAAMNRYAVLVWRGQPLTGQQQIDFAKAFGPLDLGLKKVFKRPERLEDERLIDISNVDAEGKVARRDSPKNLSNFANQLWHSDSSFQNPRAAYSMLHALVLPSWGGNTEFAELRAAYDALPERTRSEIEGLRAEHYALHTRILLGDEAYTDDQKKAIPPAVWPLAQTHPGSGRKLLFVGVHAREIIGWPVAEGRMFLSDLLEHATRREFVYTHEWQVGDLVMWDNRSTLHRGRRYDIGERRELRRTTIEDVPEATLMAA

SEQ ID NO: 126 - DNA, 882 bp, Artificial Sequence
  Synthetic polynucleotide
atgtcgtctcttgagttgacacccctgcaccctttgtttgccgcagaagctcgcgggattgacatccggcagccattgagcccatctgaagtcaagcaaataaacgccgcgatgaacaggtacgccgtgctcgtgtggcgcggacagcccctcaccggacagcagcaaatcgacttcgccaaggctttcgggcccttggatcttggacttaaaaaagttttcaagcgcccggagcgcctggaagacgaaagactcatcgacatatctaacgttgacgcggagggaaaagtggcacggagggactcaccgaaaaatcttagcaattttgcaaatcaactttggcactccgattcctcgttccaaaatccgcgcgctgcgtactcaatgctccacgcccttgtgcttcctagctggggaggaaataccgagtttgcggagcttcgcgctgcttacgatgcactcccggagcggactaggtcagagattgaggggcttcgggccgaacactatgctcttcacacccgcatattgctcggagacgaagcctatactgacgaccagaagaaggcgatcccccctgccgtttggcctcttgcccaaacccatcctggctccgggcggaaattgctcttcgtgggagtccatgcacgggaaataatcgggtggcccgtggcggagggtagaatgtttttgagcgacctgctcgaacacgcaactaggagagagttcgtctatacccatgagtggcaagttggcgatctggtcatgtgggataatagatctactctccatcgggggaggagatacgacattggtgaaagacgcgaactgagaaggacgacgattgaagatgtgcccgaggccactcttatggctgcctaa

SEQ ID NO: 127 - protein, 292 aa, Artificial Sequence
  Synthetic polypeptide, QYD64, D64
MSLQLKPLHPVFGAEASGLDLTRPLSDADVRAVNAAMNEHAVLVFRGQPLTAQQQLDFAKHFGPLDIGLKRVFKRPERLEDERLIDISNVDAQGNIARRDSPKNLSNFANQLWHSDSSFMNPRAAYSMLHCVIKPSWGGNTEFADLRHAYDTLDERTKAEIQDLKAEHFALHTRILLGDDAYTDDQKKEIPPAVWPLADTHPGSGRKVLFVGVHAREIIGWPVAEARMYLSDLLEHATRRENVYVHEWTPGDLVIWDNRSTLHRGRRYDITERRELRRTTINDTPEAMLMAA

SEQ ID NO: 128 - DNA, 879 bp, Artificial Sequence
  Synthetic polynucleotide
atgagccttcagttgaaaccccttcaccctgttttcggggcggaagcgagcggactggatcttacacgccctctgtctgacgccgacgtgagggcggttaacgccgctatgaatgagcatgcagtgttggtgttccgggggcagccgcttacagctcaacaacaactcgactttgcgaagcattttgggccgcttgatattggactcaagcgcgtctttaagagaccagaacgccttgaggacgaaagattgattgatatttcgaatgttgatgcccagggaaacatcgctcggcgcgatagcccaaagaatctgagcaactttgccaaccagttgtggcatagcgattcatcttttatgaacccccgcgctgcttacagcatgttgcattgtgtgataaagccgtcatggggcggtaacacggaatttgccgacttgagacatgcttatgataccctggacgagcggaccaaggcggaaatacaggatcttaaagccgagcattttgctcttcatacacgcattctcctgggggacgatgcttataccgacgaccaaaagaaggaaataccaccagctgtgtggccgctggccgacacgcatccgggatcgggcagaaaggtccttttcgtcggagtgcatgccagggagattattggctggcccgtggcagaggcgagaatgtacctttccgatttgctggagcacgcaaccaggagggagaacgtctatgtgcatgagtggacaccgggtgatcttgtgatctgggataatcggtcaacactccataggggacggaggtacgacattacggagcgccgcgaactcagacgcactactatcaatgatacccctgaggccatgcttatggctgcataa

SEQ ID NO: 129 - protein, 295 aa, Artificial Sequence
  Synthetic polypeptide, QYD65, D65
MAIAIRQLHPHFVGEVSGVDLRHPLTRDEVAELQAAIDHYAVLVFHDQDIGDEQQLAFALNFGDREKARGGTVTKKEDYRLTTGLNDVSNLGKDGKPLPPDHRTHLFNLGNCLWHSDSSFRPIPAKYSILSARIVNPKGGNTEFADMRAAYDALDEQTKAEIDDLVSEHSLMYSRGSLGFLDYTDEEKKMFKPVRQRLVRTHPAHGRKSLYLSSHAGAVVGMSMPEGRLLLRDLTEHATQPEFVYVHKWRLHDLVMWDNRQTVHRVRRYDQSQPRDMRRATVAGTEPTVAQQAAE

SEQ ID NO: 130 - DNA, 888 bp, Artificial Sequence
  Synthetic polynucleotide
atggccattgccattagacagcttcatccacatttcgtgggtgaggtttcgggcgtcgaccttcggcaccccctgacgagagatgaagtcgcggaattgcaggctgccatagatcactatgcagttcttgtttttcatgatcaggatatcggggacgagcagcaactcgctttcgcattgaactttggagacagagaaaaggcaaggggaggaacagttactaagaaagaggattacaggcttacgacagggctgaacgatgttagcaaccttggaaaagacggtaagcctctgcctcccgatcatcgcacacacctcttcaatttgggcaactgtctgtggcactcagactcatcttttcgccccattcccgcgaaatactctatactctcggcaagaattgtcaatccgaaaggaggtaatacagaattcgcagatatgagggcagcctatgacgctctggacgaacagactaaagctgagatagatgatctggtctccgaacactcgcttatgtattcgagagggtcacttggttttttggattatacagacgaagagaaaaagatgttcaaaccagtgcgccagaggctcgttcggacgcaccctgcacatggacgcaaaagcttgtacttgtcgtcgcatgcgggcgcagtggtcggcatgtctatgcccgaaggtcggcttcttctgagggatctgaccgaacacgctacgcagccagagtttgtgtacgtccataagtggagattgcacgacctcgtcatgtgggacaaccggcagacagtgcatcgggtgaggcggtatgatcagtcacaacccagggatatgcgccgcgcgacggtggcaggtactgaacctacggttgcccaacaggcggcagagtaa

SEQ ID NO: 131 - protein, 298 aa, Artificial Sequence
  Synthetic polypeptide, QYD66, D66
MERHAMSNGKQIVRIEPLPGKTFGAVVTGVRLSELDEDTFSLLYREWLQYALLIFPAQYLTDSQQRDAASKFGCLVEGLEAVEISNLLPTGEVRAAPDDDMMKIIRGNMQWHQDNTYMPLQAKGALFSAKRVPSSGGETGFADMRAAWDALDTETQDRLANLSAYHSLAQSQKNLGEDVKSSDSEYIGYGLDVSTVPRRSLLKIHPETDRKTLAVGRHAFGVTGMAEQESTQFVSDLIDFAVADESRTYHHIWSEGDAILWDNRCLMHRACPWNFSQPRVMLHSRIAGDPSTEAALNS

SEQ ID NO: 132 - DNA, 897 bp, Artificial Sequence
  Synthetic polynucleotide
atggagagacacgctatgtccaatggtaagcagatcgtcagaattgagcctttgcctggtaagacattcggggcggttgttaccggtgtccggctgtcagaactggacgaggacacattctcccttttgtacagggagtggctccagtatgctctccttatctttcctgcgcagtatttgactgattcccagcaacgcgatgcagcttccaaattcggatgccttgtcgagggactggaggcggtcgaaatatccaaccttctcccgacgggcgaggttagagctgcccccgatgacgatatgatgaaaatcatcagggggaacatgcagtggcaccaggacaatacctacatgccgctccaagccaaaggcgccctcttttcggcaaagagagtcccctcttcgggaggagagacaggatttgcggatatgagagctgcatgggacgctctggatactgaaacacaggatcggctggctaatctcagcgcctaccactcgcttgcgcaatcccaaaagaaccttggggaggatgtcaaatcatcagatagcgagtacattggttatgggctcgacgtgtccaccgtcccgcggcgctctcttctgaagatacaccccgagactgaccggaagactctggctgtgggaagacatgcattcggtgtgaccggaatggctgagcaggaatccacccaattcgtttcagacctcatagattttgccgtggccgacgagtcacgcacttaccaccacatctggagcgaaggggacgctattctttgggacaataggtgcctcatgcacagagcttgtccgtggaacttctctcagccgcgggtcatgttgcactcacggatagcaggcgacccaagcactgaggccgcgctgaactcgtaa

SEQ ID NO: 133 - protein, 292 aa, Artificial Sequence
  Synthetic polypeptide, QYD67, D67
MSPAFDIAPLDATFGAVVTGVHLATLDDAGFAALHAAWLQHALLIFPGQHLSNDQQITFAKRFGAIERIGGGDIVAISNVKADGTVRQHSPAEWDDMMKVIVGNMAWHADSTYMPVMAQGAVFSAEVVPAVGGRTCFADMRAAYDALDEATRALVHQRSARHSLVYSQSKLGHVQQAGSAYIGYGMDTTATPLRPLVKVHPETGRPSLLIGRHAHAIPGMDAAESERFLEGLVDWACQAPRVHAHQWAAGDVVVWDNRCLLHRAEPWDFKLPRVMWHSRIAGDPASETALAH

SEQ ID NO: 134 - DNA, 879 bp, Artificial Sequence
  Synthetic polynucleotide
atgtcacccgctttcgatattgctcctctcgatgcaacatttggagcggttgttacgggcgttcacctcgctactcttgacgatgcgggctttgcagctttgcatgctgcatggctccaacatgcgctgttgatattcccgggtcagcacctttcgaatgaccagcaaatcacctttgctaagcggttcggtgcaatcgagaggatcggcgggggcgacatagtcgctatctctaatgtcaaggctgacggaactgttcgccagcattccccagcagaatgggacgatatgatgaaagttatagtgggtaacatggcttggcatgctgattctacttacatgccggtcatggcccagggcgcagtgttctcggcagaagtggttccagctgtgggaggtcggacttgcttcgcggatatgcgcgcggcttacgatgctctcgatgaagcaacgagggccttggtgcatcaacggagcgcgcgccattcattggtgtattcccaatctaagctcggacacgtgcaacaggctggcagcgcctacattggctacggcatggatacaactgccacgcccctgcgccccctcgttaaagttcaccccgaaaccggtcgcccctcattgttgattggccggcacgcccacgcaattccgggaatggatgccgccgagtctgaaagatttcttgagggtttggtggactgggcgtgccaagccccccgggttcatgcccaccaatgggcagccggtgatgtcgttgtgtgggacaatcggtgtcttcttcatagggctgaaccgtgggattttaaactcccgagggtgatgtggcactcccgcatagcgggcgatccggcttccgagaccgccctggcacattaa

SEQ ID NO: 135 - protein, 292 aa, Artificial Sequence
  Synthetic polypeptide, QYD68, D68
MSPAFDIAPLDATFGAVVTGVKLADLDDAGWLDLQAAWLQHALLIFPGQHLSNDQQITFAKRFGAIERIGGGDIVAISNVKADGTVRQHSPAEWDDMMKVIVGNMAWHADSTYMPVMAQGAVFSAEVVPAVGGRTCFADMRAAYDALDEATRALVHQRSARHSLVYSQSKLGHVQQAGSAYIGYGMDTTATPLRPLVKVHPETGRPSLLIGRHAHAIPGMDAAESERFLEGLVDWACQAPRVHAHQWAAGDVVVWDNRCLLHRAEPWDFKLPRVMWHSRLAGRPETEGAALV

SEQ ID NO: 136 - DNA, 879 bp, Artificial Sequence
  Synthetic polynucleotide
atgtcacctgcttttgatattgccccgcttgacgctacgttcggagctgttgtcacaggagtcaagctcgctgatttggatgatgcggggtggttggatctgcaggccgcctggcttcaacacgcccttcttatctttccaggacagcacctttcaaacgaccaacaaattacctttgctaaacggtttggagccatcgagaggattggagggggagatatcgttgctatatcaaacgtgaaagccgacgggacagttcggcagcactctcccgccgagtgggatgatatgatgaaggtgattgtggggaatatggcatggcacgcggactcgacttatatgccggttatggcgcaaggggctgtcttctcggccgaggtcgttcccgccgtgggagggcgcacgtgttttgcagatatgagagcggcctatgacgcactggacgaggcaactagagcactggtccatcagcgcagcgccaggcactcactggtgtactctcaaagcaaactggggcatgttcaacaggctggctctgcgtacatcggatatggtatggacaccacagcgacgccactccggcctcttgttaaagtccaccccgaaaccggtcggccatctttgctcatcgggagacatgcccatgccatccctggtatggacgccgctgaatccgaaagatttctggaagggttggtcgactgggcttgtcaggccccacgggtgcatgctcaccaatgggcggctggtgacgttgtggtctgggacaaccgctgccttctccacagagctgaaccatgggatttcaagctcccgagagtgatgtggcactcgagactcgcggggcgcccagagactgaaggcgcagcgctcgtctaa

SEQ ID NO: 137 - protein, 293 aa, Artificial Sequence
  Synthetic polypeptide, QYD69, D69
MAQTTLQITPTGATLGATVTGVHLATLDDAGFAALHAAWLQHALLIFPGQHLSNDQQITFAKRFGAIERIGGGDIVAISNVKADGTVRQHSPAEWDDMMKVIVGNMAWHADSTYMPVMAQGAVFSAEVVPAVGGRTCFADMRAAYDALDEATRALVHQRSARHSLVYSQSKLGHVQQAGSAYIGYGMDTTATPLRPLVKVHPETGRPSLLIGRHAHAIPGMDAAESERFLEGLVDWACQAPRVHAHQWAAGDVVVWDNRCLLHRAEPWDFKLPRVMWHSRLAGRPETEGAALV

SEQ ID NO: 138 - DNA, 882 bp, Artificial Sequence
  Synthetic polynucleotide
atggcgcagactaccttgcaaataacgccgactggtgcgaccctcggggctacggtgacgggagttcatcttgccacccttgacgatgctggcttcgctgctcttcatgctgcatggctgcagcacgcccttttgatctttccagggcagcatctttcaaacgaccagcagattacgtttgccaaaagatttggagcgatcgaacgcattggaggaggggatatagtcgcaatttcaaatgtcaaggcagatggaactgtcaggcaacattctccggcggagtgggatgacatgatgaaagtgatagtgggaaacatggcgtggcatgctgactccacttatatgccggtcatggcccagggtgcagtgttttccgctgaggtggttcctgcagttggtggccggacttgcttcgctgacatgagagctgcatacgacgctttggatgaagcgacgagggctttggtccatcagagatcagcgaggcattctcttgtctactctcaatcaaaactcgggcatgttcaacaggccgggtcagcctatatcggctatggtatggacaccaccgctacaccactgagaccgttggtgaaggttcatccggaaacgggaagaccgtcccttctgatcggaaggcacgctcatgcaatacctggcatggacgccgcagaatccgagaggttccttgaagggttggtcgattgggcatgtcaggcaccacgggtccacgcccatcaatgggctgccggcgacgtggtggtttgggacaaccgctgtcttttgcatagagccgaaccttgggactttaaactgcccagagtcatgtggcactctaggctcgccggtagaccagaaaccgaaggtgcggcattggtctaa

SEQ ID NO: 139 - protein, 295 aa, Artificial Sequence
  Synthetic polypeptide, QYD70, D70
MHAALTPLTNKYRHIDVQPLTGVLGAEITGVDLREPLDDSTWNEILDAFHTYQVIYFPGQAITNEQHIAFSRRFGPVDPVPILKSIEGYPEVQMIRREANESSRYIGDDWHADSTFLDAPPAAVVMRAIEVPEYGGDTGFLSMYSAWETLSPTMQATIEGLNVVHSATKVFGSLYQATNWRFSGTSVKVMDVDAGDRETVHPLVVTHPVTGRRALYCNQVYCQKIQGMTDAESKSLLQFLYEHATQFDFTCRVRWKKDQVLVWDNLCTMHRAVPDYAGKFRYLTRTTVAGDKPSR

SEQ ID NO: 140 - DNA, 888 bp, Artificial Sequence
  Synthetic polynucleotide
atgcatgccgctctcacacctcttactaacaaatatagacatatcgacgtgcagccccttacaggggtgttgggtgctgagatcactggtgtggatctcagggaacctttggacgattctacatggaacgaaatactcgacgcatttcatacctaccaggttatatactttccgggacaagcgataaccaatgagcagcacattgctttctctaggcgcttcggtcctgtggacccggtgcccattctgaaaagcatcgaaggttaccccgaggtccagatgataaggagggaggctaacgagtctagccggtacatcggtgatgactggcacgccgactcaacgtttttggacgctcctcctgctgcggttgtgatgagggcgatcgaagtccctgaatatggcggcgacacgggattcctctcaatgtactcagcctgggagacactgtccccgactatgcaagctaccatcgagggcttgaacgttgtccactccgcgacgaaagtgtttgggagcctctaccaggcgaccaactggcgcttttctggcacgtctgtgaaggttatggatgttgacgctggggacagagaaacagtccatccgttggtcgtgacacatccagtgactgggaggagggcgttgtactgtaatcaggtttattgccagaagatccaagggatgacagacgccgagagcaaatccctgttgcaatttctttatgaacatgcgacccaatttgattttacctgccgcgtcagatggaaaaaagatcaggtcctcgtttgggataatttgtgtaccatgcatcgcgcagtccccgattatgcgggcaagttcagatatctgacaagaactacagtggccggtgacaagccatctaggtaa

SEQ ID NO: 141 - protein, 296 aa, Artificial Sequence
  Synthetic polypeptide, QYD71, D71
MPFQVNQLHPIFAAEMFGCDILKPVTDETRNAVEDAMAKYAVLVIRDQAAASDEDQVRFAKAFGPLELPPDLGMSERTRPTRVHPKLYDVSNLDENGNLDKPDTLRRKFAKGNELFHTDSSFNDLPTKWSMLLAHVVTPTGGNTEFVDTRAAYDALSPSMKEQVEPLSVIHSLTYSRERGGLTGTTVFDRAFPPVTQPLVRTSASGRKALYIGAHAATVVGMDEAAGRTLLDKLISDASRPEYIYSHKWLPGDLVIWDNRCTMHRATEYAYMEDRRDLRRATINEFGEDRVGARPI

SEQ ID NO: 142 - DNA, 891 bp, Artificial Sequence
  Synthetic polynucleotide
atgcctttccaggtgaaccaacttcacccaatctttgcggctgaaatgttcggttgcgacattttgaaacccgttacagacgaaactcgcaacgctgttgaggacgcaatggccaagtatgccgtgcttgttatacgcgaccaagcagctgcgtccgatgaagaccaggtgcggttcgccaaagcattcggtccgcttgaactcccaccggatctggggatgagcgaacgcacacggccaactcgggtgcatccgaagctgtatgatgtgtcaaatttggatgaaaacggaaatcttgacaagcccgacacactgcgccggaagtttgcaaaagggaacgaacttttccataccgactcgtctttcaatgacctccccacgaaatggtccatgctcctggcccacgttgtcacgcctacagggggaaacacagagttcgttgacacgcgcgcggcatatgatgcactctcgccgtctatgaaagagcaggtcgagccactctcggttattcattctctgacttattctcgggagagaggaggactcaccggtactacagttttcgaccgggccttccctcccgttacgcaacctcttgttagaacaagcgcaagcggacggaaagccctctatataggcgcgcacgctgcaactgttgtggggatggacgaggctgctggtcggactctcttggataagttgatctcggacgcgagcagaccggagtacatctatagccacaagtggctgccaggagatctcgtcatatgggacaaccggtgcaccatgcatcgcgcaacggagtacgcgtatatggaggacagacgcgatctcaggcgcgcgaccattaatgaatttggcgaagacagggtcggagcccggcccatctaa

SEQ ID NO: 143 - protein, 288 aa, Artificial Sequence
  Synthetic polypeptide, QYD72, D72
MAADAPFVVKPLPASFGAVVTGLALADLDEAAFAALYRTWLDYALLIFPGQHLDRDQQIAFARRFGPLEFDIAPISNVRSDGSVRPDDDADDVVKVLKGNMGWHCDSTYMPVQAKGAVFTAHVVPEDGGETDWADMRAAYDALDPAMRRRIEGLSAYHSLRHSQGKLGHVHKPGSAYSGYGMTVEAPPLRPLVKVHPETSRKTLMVGRHAYGIPGFEDAESERLLDELADFACRPPRTWRHSWTPGDAVIWDNRALMHRACPWDMTRPRVMYHSRLAGDPVAEFAAPA

SEQ ID NO: 144 - DNA, 864 bp, Artificial Sequence
  Synthetic polynucleotide
atggcagctgacgcgccttttgtggtcaaacctcttccagcctcttttggtgctgtggtcactgggctcgctctggccgatttggatgaggcggcttttgcagcgttgtatcggacttggctcgactacgctctgctgattttcccaggccagcaccttgatcgggaccagcagattgccttcgctaggcgcttcgggcctctcgaatttgacatagcccccatcagcaatgttcgctcggatggttcagttaggcctgatgacgacgcggatgatgtcgtcaaagttctcaaagggaatatgggctggcattgtgactctacctacatgccggtccaagcgaagggggccgtcttcacggcgcatgtggtccctgaagacgggggagaaaccgactgggctgatatgagggcagcatatgacgctcttgacccggcaatgagaaggaggattgaaggtttgagcgcttaccactcactgagacacagccaaggcaagctgggacatgttcacaagccaggttccgcatattcggggtacggtatgacggttgaagccccaccactcagacccttggtgaaagtgcacccagaaacatcccggaagaccctcatggtcgggaggcatgcgtatggcatacccgggttcgaagacgcagagtcagaacggttgttggacgagttggcggattttgcctgtaggccaccccggacatggcggcattcttggacaccgggtgatgctgtgatatgggataatagagcgttgatgcaccgcgcgtgcccctgggatatgactaggcccagggttatgtatcattctcgcctggccggtgatccagtcgcggaattcgcggcaccagcc

SEQ ID NO: 145 - protein, 285 aa, Artificial Sequence; identical to SEQ ID NO: 111
  Synthetic polypeptide, QYD73, D73

SEQ ID NO: 146 - DNA, 855 bp, Artificial Sequence
  Synthetic polynucleotide
atggcaagaactttcgacgtgcaacctcttgatgcgacattcggggcggtgatttccggtgttaaactggcggctcttgacgatgaaacttggcgggaactgtacgcgacgtggcttcaatacgccctgctcgtgtttccgggtcagcacctgagcagggaccaacagattgctttcgcgagacgctttgggccactcgaatttgaaataatcgcactgagcaacgtcaaatcggacggtactattcggtctgaggtggagaatgacgatgttattaaggtgttgaaaggcaatatgggatggcacgcagactccacgtacatgcccgtccaagctaaaggtgctgtgtttactgccgaggtggttccctcggtggggggtcaaactggttgggcagatatgagggcggcatacgacgcacttgatggcgctaccaggcagaaggttgaacagctttcagcttatcactcattgcactattcacaatccaagctcggacatcagcacaagccagggagcgcctatagcggatatggatttcacgacggcccggtccccctgcgcccccttgtcaaaacccatccggagactggccgcaagagcttgttgattggcagacacgcgcataacattcctggcatggacaaggatgagtctgcacggttccttgaagatcttgtggcttccgcttgtcaaccaccccggatttaccatcatacttgggctgctggggacgctgttgtttgggataaccggtgtcttctccatcaggccactccctgggatatgacccaaccccgggtgatgtggcactcgaggatagctggcgatccactttccgaagccgcgctcggg

SEQ ID NO: 147 - protein, 284 aa, Artificial Sequence
  Synthetic polypeptide, QYD74, D74
MARAFDIEPLDATFGAIITGVKLAALDDAVWRDLYAAWLEYALLIFPSQNLGRDEQIAFAKRFGALEFELAPISNVNSDGTLITDPDNDILKVLKGNMGWHSDSTYMPIQAKGAVFSARVVPKSGGSTGWADMRAAYDALDDNLKRKVERLAAHHSLRYSQAKLGHFPKKHSAYSGYGFHDGPEPLRPLVKTHPETGRKSLLIGRHAHNIPGMSAEESERFLQELVDFACRPPRVYHHEWTAGDVAVWDNRCLLHQATPWDMSEPRVMWHSRIAGDPTTESALA

SEQ ID NO: 148 - DNA, 852 bp, Artificial Sequence
  Synthetic polynucleotide
atggcgagggccttcgatatagagcccttggatgcaacgttcggcgccataataactggggtgaaactcgccgctctcgatgacgcggtttggcgggatctctacgccgcctggctggagtacgcactgctgatcttcccttctcaaaatctcggcagagatgagcaaatagcattcgcaaaacggtttggtgcgttggagtttgaattggccccgattagcaacgtcaatagcgatggtactctcataacagatcccgataatgacatactcaaggtgctgaagggtaacatgggatggcattcggattcaacgtacatgcccatacaagcaaaaggagctgtgttctcggcaagggttgtgccaaagtcgggtggatcgacgggatgggcggacatgcgggcagcttatgatgcgctcgacgacaacttgaaacggaaggtcgaaagattggccgcgcaccactctctcagatactctcaggccaaactgggccatttcccaaaaaaacattcggcgtattctggatacggttttcacgatgggccagaacccctcagaccgctcgtgaagactcatcctgaaaccgggagaaaatccctcttgataggcaggcacgctcacaatattccaggaatgtcggcagaggaatcggaaagattcttgcaagaacttgtggatttcgcatgtcgcccacctagagtctatcatcacgaatggactgcgggtgatgttgcggtttgggacaatcgctgcctgttgcatcaggcaaccccctgggacatgtctgagccgagagttatgtggcactctcgcattgccggggatccaaccactgagtctgccctcgca

SEQ ID NO: 149 - protein, 284 aa, Artificial Sequence
  Synthetic polypeptide, QYD75, D75
MPSFNIAPIDASFGAVVTGIRLATVDDERWRELHQAWLEHALLIFPGQHLSRDEQISFARRFGPLEFDMAAISNVKSDGTLRIEADNDDVVKILKGNMGWHADSTYMSVQAKGAVFSAEVVPAAGGQTGFADMRAAYDALDDGLRTKVETLAAHHSLHYSQAKLGHQPEAGSAYSGYGFHAGPVPLRPLVKTHPETGRKSLLIGRHAHNIPGLDPDESERLLDELVDFACQAPRVYHHSWSAGDVVIWDNRCLLHRATPWDMTQPRVMWHSRIAGDPVAEASLN

SEQ ID NO: 150 - DNA, 852 bp, Artificial Sequence
  Synthetic polynucleotide
atgccctccttcaacatagctccgattgatgcgagcttcggtgcagttgtcaccggcatcaggcttgcaacagtcgacgacgagaggtggcgcgagctgcatcaggcttggctggaacatgcccttctcatcttccctggtcagcatctgtccagggacgaacagatttcattcgcaaggagattcggcccgctcgaattcgacatggcggccattagcaacgtgaagtccgacggaactcttcggattgaagccgacaacgatgatgtggttaagatccttaaaggtaatatgggatggcatgccgactctacatatatgtcagtccaagcgaagggtgccgttttctcggccgaagtggttcccgctgccgggggacagacaggttttgctgatatgcgggccgcgtacgacgcacttgatgacggcttgcggactaaagtggaaacacttgctgcgcatcactcgcttcactactcccaggcaaagttggggcaccaacctgaggcgggatcggcctactctggttatggtttccatgccggccctgttcctctgcggcctctcgtgaaaacccaccctgagactggccgcaaaagcttgcttatcggtagacatgcgcataacataccaggattggaccctgacgagtctgaaaggttgttggatgaattggttgatttcgcctgtcaggccccgagagtgtatcaccactcgtggtcagctggtgatgttgttatctgggataatcggtgtcttctccacagggccaccccctgggacatgacacagccaagggttatgtggcactccagaattgcaggggacccggttgccgaggcttcgctcaac

SEQ ID NO: 151 - protein, 285 aa, Artificial Sequence; identical to SEQ ID NO: 107
  Synthetic polypeptide, QYD76, D76

SEQ ID NO: 152 - DNA, 855 bp, Artificial Sequence
  Synthetic polynucleotide
atggccaggatgttcgatattgctccgttggacgctaccttcggagcggtcgttactggagtgaaggttacgagaatcgatgatgcaacgtggagggatctccacgcggcctggttggagtacgcgctcctgatatttccgggtcaatttctttctagagccgaacagattgcttttgccaaaagatttggtccgttggaatttgaaatggcccccatctctaacgttaaggctgatggcacaattagaatcgaagccgacaatgatgacgttgtcaaggtcctgaaaggtaatatgggatggcacgcagattcaacatacatgcccgttcaggcaaagggagcggtcttttctgcagaagtcgttcctggtgaaggtgggcggacgggatgggctgatatgagggcggcttatgatgcattggacgcggccaggagagtccgcgtggagtcactgacagcatatcactctctgtattattctcaagcaaaactcggtcaccagcctaaaaggggctcggattacagcggttatggtttccacgacgggcctgtgccaagacggccgttggtgaaggttcatccagaaacgggtagaaaatctttgctcattgggcggcacgcacataacattcccggtatggatgtggcggaatctacccaatttctcgaatcacttgtggactttgcctgtcagccgcctcggatctatcatcatcaatggacagcaggagacgctgttgtgtgggacaaccggtgtcttttgcatcgggcgactccttgggatatgactaagccacgcgtcatgtggcactctcgcatcgcaggagatcccgtcagcgaagcatctctcggt

(19) United States
(12) Patent Application Publication
(10) Pub. No.: US 2024/0043859 A1
(43) Pub. Date: Feb. 8, 2024

(54) HERBICIDE-RESISTANT PROTEIN, AND GENE AND USE THEREOF

(71) Applicant: QINGDAO KINGAGROOT CHEMICAL COMPOUND CO., LTD., Qingdao, Shandong (CN)

(72) Inventors: Huarong LI, Qingdao, Shandong (CN); Wei QI, Qingdao, Shandong (CN); Guizhi LIU, Qingdao, Shandong (CN); Qiqi HOU, Qingdao, Shandong (CN)

(21) Appl. No.: 18/019,498

(22) PCT Filed: Aug. 4, 2021
(86) PCT No.: PCT/CN2021/110411

§ 371 Date: Feb. 3, 2023

(30) Foreign Application Priority Data
Aug. 6, 2020 (CN) 202010784387.X

(51) Int. Cl. C12N 15/82; C12N 9/02

(52) U.S. Cl. CPC C12N 15/8274; C12N 9/0069

(57) ABSTRACT

The present invention belongs to the field of biotechnology, and in particular relates to a protein and gene that can confer resistance to hormone herbicides and ACCase inhibitor herbicides, and the use thereof, as well as a plant, seed, cell and plant part with herbicide tolerance and the method of application thereof.

### TECHNICAL FIELD

The present invention belongs to the field of biotechnology, and in particular relates to a herbicide-resistant protein, and gene and use thereof.

### BACKGROUND OF THE INVENTION

Weeds can quickly deplete moisture in the soil and nutrients needed by various crops, and compete with crops for growing space, severely affecting growth and development of crops. With the development of rural urbanization and the transfer of rural labor force, manual weeding is no longer economical or realistic. Changes in the cultivation system, such as the promotion and implementation of light and simplified cultivation methods including natural zero-tillage, crop direct seeding, etc., have caused more and more serious issues of weed damage, severely restraining the high and stable yield of crops, and solutions are urgently needed. In this context, the use of herbicides has become an inevitable choice, and weeding for arable land and bare place is increasingly dependent on the use of herbicides at the moment.

Herbicides can be classified into many types according to different mechanisms of weed control. However, according to their selectivity, herbicides can be simply divided into two categories, biocidal herbicides and selective herbicides. The exceptionally popular herbicides, such as glyphosate, 2,4-D, glufosinate, paraquat, etc. are all biocidal herbicides. These biocidal herbicides are mostly used for weed control in bare place, railways, highways, warehouses, forest firebreaks, etc. With the emergence and popularization of transgenic herbicide-resistant crops, some biocidal herbicides are also widely used for weed control in farmlands, for example, glyphosate, 2,4-D and glufosinate are used for weeding in fields of herbicide-resistant maize, soybean, cotton, sugar beet and the like without harming the crops.

Glyphosate has been widely used worldwide for more than 25 years, thus leading to over-reliance on glyphosate and glyphosate-tolerant crop technologies. The long-term use of a single herbicide glyphosate has exerted high selection pressure on wild weed populations, resulting in the occurrence of resistant weeds. Over 40 species of weeds have been reported to show resistance to glyphosate, Pest Manag Sci 2018; 74: 1089-1093, including broad-leaved and gramineous weeds, such as a variety of Amaranthus spp., Ambrosia spp., Conyzacanadensis, Eleusine indica, Salsola tragus, etc.

(2,4-dichlorophenoxy)acetic acid (2,4-D) is a representative of hormone herbicides which are another type of biocidal herbicides that can be used in combination with glyphosate or alone to control glyphosate-resistant weeds. 2,4-D has been used for wide spectrum broad-leaved weed control in arable land and bare place for more than 65 years, while still widely used worldwide. On the other hand, 2,4-D has particularly poor selectivity for dicotyledons (such as soybean or cotton), and is generally not used for weed control on sensitive dicotyledons; it is not very safe even for gramineous crops. These factors also limit the application of 2,4-D in farmland weed control.

2,4-D has an advantage that is a shorter half-life in the soil and has no significant impact on succession crops. Based on this clue, scientists have found many soil microorganisms capable of degrading compound 2,4-D. Ralstonia eutropha has been extensively studied for degrading 2,4-D with a degradation gene called tfdA. The tfdA encodes AADs, α-ketoglutarate dependent aryloxyalkanoate dioxygenases. The type of enzyme can degrade phenoxyacetic acid herbicides, including 2,4-D, MCPA, etc. and eliminate the aceticoceptor from 2,4-D to generate 2,4-dichlorophenol (DCP) with lower toxicity. The TfdA protein has a TauD domain responsible for degrading 2,4-D. The reported 2,4-D degradation is mainly through two metabolic pathways:

1. Under the action of the α-ketoglutarate dependent aryloxyalkanoate dioxygenases gene tfdA (derived from Alcaligenes eutrophus JMP 134) or CadABC (derived from Bradyrhizobium HW13), the aceticoceptor is eliminated from 2,4-D to generate 2,4-dichlorophenol (DCP) which is 100 times less toxic than 2,4-D under catalysis. Subsequently, 2,4-dichlorophenol is converted into 3,5-dichlorocatechol under the catalysis of hydroxylase TfdB, and then its ring is opened under the action of TfdC, TfdD, TfdE and TfdF, and finally degraded into β-ketoadipic acid, which enters the tricarboxylic acid cycle.

2. In Azotobacter chroococcum, 2,4-D is deprived of a chlorine atom first to generate p-chlorophenoxyacetic acid under the action of an unknown dechlorinase, and then oxidized to eliminate the aceticoceptor to generate p-chlorophenol, followed by further hydroxylation to produce 4-chloropyrocatechol, which is then degraded into β-ketoadipic acid following by ring opening, and finally enters the tricarboxylic acid cycle. Some AADs, for example, AAD-12 from Comamonas acidovorans can catalyze other herbicides, such as chlorofluoropyridyloxyacetic acids (Triclopyr and Fluroxypyr) of the pyridyloxyacetic acids and aryloxyphenoxypropionate (AOPP) herbicides (CN101688219B; US2019/0017066A1; U.S. Ser. No. 10/167,483B2; Anne Westendorf, Dirk Benndorf, Roland H. Müller, Wolfgang Babel. 2002. The two enantiospecific dichlorprop/α-ketoglutarate-dioxygenases from Delftia acidovorans MC1-protein and sequence data of RdpA and SdpA. Microbiol. Res. (2002) 157, 317-322, http://www.urbanfischer.de/journals/microbiolres; Jonathan R. Chekan, Chayanid Ongpipattanakul, Terry R. Wright, Bo Zhang, J. Martin Bollinger Jr., Lauren J. Rajakovich, Carsten Krebs, Robert M. Cicchillo, and Satish K. Nair. 2019. Molecular basis for enantioselective herbicide degradation imparted by aryloxyalkanoate dioxygenases in transgenic plants).

With the development of transgenic technology, genes encoding AAD enzymes have been transformed into sensitive plants such as cotton and tobacco, and the transgenic plants have acquired resistance to 2,4-D, which provides technical solutions for solving glyphosate-resistant weeds. Dow AgroSciences LLC (the U.S.) has launched transgenic maize, soybean, cotton and other crops, respectively expressing AAD1, AAD12 and AAD13 to degrade 2,4-D herbicides (T. R. Wright et al., Robust crop resistance to broadleaf and grass herbicides provided by aryloxyalkanoate dioxygenase transgenes. Proc. Natl. Acad. Sci. U.S.A. 107, 20240-20245 (2010)), which provides conditions for the application of hormone herbicides represented by 2,4-D in farmland weeding.

In summary, there are various types of herbicides whereas the number of degradation genes screened so far and their resistance spectrums are very limited, which cannot meet the demands for cultivating herbicide resistance traits in crops. It is urgently needed to screen more types of genes to provide genetic resources for the research and development of transgenic crops.

### BRIEF SUMMARY OF THE INVENTION

In order to solve the above problems existing in the prior art, the present invention provides a herbicide-resistant protein, and gene and use thereof.

The present invention provides a recombinant DNA molecule comprising a nucleic acid sequence selected from:
(1) a nucleic acid sequence encoding a protein or a bioactive fragment thereof that comprises an amino acid sequence having at least 90%, at least 95%, at least 96%, at least 97%, at least 98%, at least 99% or 100% sequence identity with at least one amino acid sequence selected from the following group: SEQ ID NO: 41, SEQ ID NO: 51, SEQ ID NO: 59, SEQ ID NO: 63, SEQ ID NO: 65, SEQ ID NO: 67, SEQ ID NO: 69, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 123, SEQ ID NO: 143, SEQ ID NO: 145, SEQ ID NO: 147, SEQ ID NO: 149 and SEQ ID NO: 151, or a complementary sequence thereof
(2) a nucleic acid sequence as set forth in: SEQ ID NO: 42, SEQ ID NO: 52, SEQ ID NO: 60, SEQ ID NO: 64, SEQ ID NO: 66, SEQ ID NO: 68, SEQ ID NO: 70, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 124, SEQ ID NO:144, SEQ ID NO: 146, SEQ ID NO: 148, SEQ ID NO: 150 or SEQ ID NO: 152, or a complementary sequence thereof
(3) a nucleic acid sequence that hybridizes to the sequence as shown in (1) or (2) under stringent conditions; or
(4) a nucleic acid sequence encoding the same amino acid sequence as the sequence shown in (1) or (2) due to degeneracy of genetic code, or a complementary sequence thereof.

In a specific embodiment, the recombinant DNA molecule is operably linked to a heterologous promoter functional in a plant cell.

The present invention provides a DNA construct comprising a heterologous promoter that is functional in a plant cell and operably linked to the recombinant DNA molecule.

In another specific embodiment, the DNA construct exists in the genome of a transgenic plant.

The present invention provides a protein or a bioactive fragment thereof encoded by the recombinant DNA molecule.

The present invention provides another protein or a bioactive fragment thereof, of which the amino acid sequence has at least 90%, at least 95%, at least 96%, at least 97%, at least 98%, at least 99% or 100% sequence identity with at least one amino acid sequence selected from the following group: SEQ ID NO: 41, SEQ ID NO: 51, SEQ ID NO: 59, SEQ ID NO: 63, SEQ ID NO: 65, SEQ ID NO: 67, SEQ ID NO: 69, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 123, SEQ ID NO: 143, SEQ ID NO: 145, SEQ ID NO: 147, SEQ ID NO: 149 and SEQ ID NO: 151.

In a specific embodiment, the protein or a bioactive fragment thereof has oxygenase activities on at least one of the following types of herbicides: hormone herbicides, ACCase inhibitor herbicides.

The present invention provides a plant, seed, cell or plant part comprising the recombinant DNA molecule, the DNA construct, or the protein or a bioactive fragment thereof.

In a specific embodiment, the plant, seed, cell or plant part has tolerance to at least one of the following types of herbicides: hormone herbicides, ACCase inhibitor herbicides.

The present invention provides an isolated polynucleotide comprising the recombinant DNA molecule, the DNA construct, or a nucleic acid sequence encoding the protein or a bioactive fragment thereof, or the complementary sequence of the nucleic acid sequence.

The present invention provides a plant genome comprising the polynucleotide.

The present invention provides a vector comprising the polynucleotide and a homologous promoter operably linked thereto.

In a specific embodiment, the promoter is an inducible promoter or a promoter of the gene itself in the plant genome.

The present invention provides a host cell comprising the polynucleotide or the vector.

The present invention provides a method for producing or improving a plant or seed with herbicide tolerance, comprising transforming a plant cell or tissue with the recombinant DNA molecule or the DNA construct, and regenerating a herbicide-tolerant plant from the transformed plant cell or tissue.

The present invention provides a plant or seed produced through the above-mentioned method.

The present invention provides a method for conferring herbicide tolerance on a plant, seed, cell or plant part, comprising expressing the protein or a bioactive fragment thereof in the plant, seed, cell or plant part;

or, comprising crossing a plant that expresses the protein or a bioactive fragment thereof with another plant, and screening for a plant, seed, cell or plant part capable of producing or improving herbicide tolerance;

or, comprising gene editing plant, seed, cell or plant part to achieve expression of the protein or a bioactive fragment thereof.

In a specific embodiment, the plant, seed, cell or plant part comprises the DNA construct.

The present invention provides a use of the recombinant DNA molecule, the DNA construct, the protein or a bioactive fragment thereof, the polynucleotide, the plant genome, the vector or the host cell for producing or improving herbicide tolerance of a plant, seed, cell or plant part.

The present invention provides a method for controlling weeds in a plant growing area, comprising exposing a plant growing area, where a herbicide-tolerant plant or seed grows, to the herbicide. The plant or seed comprises the above-mentioned plant or seed, a plant or seed prepared by the above-mentioned method, or a plant or seed comprising the recombinant DNA molecule.

### BRIEF DESCRIPTION OF THE SEVERAL VIEWS OF THE DRAWING(S)

FIG. 1 shows AAD-12 expressing bacteria Escherichia coli degrading 2,4-D in bacteria to generate phenolic substances which appear red after adding a chromogenic solution.

FIG. 2-1 shows the degradation spectrum testing in bacteria of 16 screened new AAD-like gene expressing bacteria (No. D42, D43, D44, D45, D46, D47, D48, D49, D50, D30, D72, D73, D74, D75, D76 and D58, D=QYD) capable of degrading 2,4-D. The tested compounds include 2,4-D, MCPA, 2,4-DB, dicamba, fluroxypyr, triclopyr and quizalofop-P-ethyl which is a ACCase inhibitor. The results show that the AAD-like enzymes encoded by these genes also have strong ability to degrade MCPA in addition to 2,4-D; among which D42, D43, D46, D47, D48, D30, D72, D73, D74, D76 and D58 (D=QYD) show slight degradation to triclopyr; do not degrade the others. The blank vector pET15b serves as a negative control; AAD12 is a positive control.

FIG. 2-2 shows the degradation spectrum testing in bacteria of the other 9 screened new AAD-like gene expressing bacteria (No. D21, D51, D52, D53, D54, D55, D56, D57, D62, D=QYD) capable of degrading 2,4-D. The tested compounds include 2,4-D, MCPA, 2,4-DB, dicamba, fluroxypyr, triclopyr and quizalofop-P-ethyl which is a ACCase inhibitor. The results show that the AAD-like enzymes also have strong ability to degrade MCPA in addition to 2,4-D; but do not degrade the others. The blank vector pET15b serves as a negative control; AAD12 is a positive control.

FIG. 3 shows a representative vector for overexpression of AAD-like genes in Arabidopsis thaliana. The expression of AAD-like genes is regulated by the pAtUBQ10 (=AtUbi10 promoter) Arabidopsis thaliana ubiquitin promoter and the mannopine synthase (MAS) terminator. Hygromycin resistance gene (HygR=hpt) serves as the selection marker.

FIG. 4-1 shows a representative 2,4-D resistance screening testing of the T1 generation seedlings of the transgenic Arabidopsis thaliana transformed with an AAD-like gene. The plate medium comprises compound 2,4-D (0.3 μmol). Col-0: wild-type Arabidopsis thaliana Columbia-0 variety, whose seminal roots are inhibited under the action of 2,4-D and not capable of growing. pQYxxxx represents the T1 generation seedlings transformed by flower dipping with various overexpression vectors: The seminal roots of the plants that are not normally transformed (i.e., wild-type), or that are normally transformed but whose transgenes are not resistant to 2,4-D, also fail to grow. Those plants with seminal roots growing normally are indicated as transgenic and resistant to 2,4-D.

FIG. 4-2 shows a 2,4-DB sodium resistance screening testing of the T1 generation seedlings of the transgenic Arabidopsis thaliana transformed with an AAD-like gene. The plate medium comprises compound 2,4-DB sodium (0.7 μmol). Col-0: wild-type Arabidopsis thaliana Columbia-0 variety, whose seminal roots are inhibited under the action of 2,4-DB sodium and not capable of growing. pQYxxxx represents the T1 generation seedlings transformed by flower dipping with various overexpression vectors: The seminal roots of the plants that are not normally transformed (i.e., wild-type), or that are normally transformed but whose transgenes are not resistant to 2,4-DB sodium, also fail to grow. Those plants with seminal roots growing normally are indicated as transgenic and resistant to 2,4-DB sodium.

FIG. 4-3 shows a MCPA resistance screening testing of the T1 generation seedlings of the transgenic Arabidopsis thaliana transformed with an AAD-like gene. The plate medium comprises compound MCPA (0.1 μmol). Col-0: wild-type Arabidopsis thaliana Columbia-0 variety, whose seminal roots are inhibited under the action of MCPA and not capable of growing. pQYxxxx represents the T1 generation seedlings transformed by flower dipping with various overexpression vectors: The seminal roots of the plants that are not normally transformed (i.e., wild-type), or that are normally transformed but whose transgenes are not resistant to MCPA, also fail to grow. Those plants with seminal roots growing normally are indicated as transgenic and resistant to MCPA.

FIG. 5-1 shows a 2,4-D resistance testing of the T2 generation seedlings of the transgenic Arabidopsis thaliana transformed with an AAD-like gene. The plate medium comprises compound 2,4-D (0.3 μmol). Col-0: wild-type Arabidopsis thaliana Columbia-0 variety, whose seminal roots are not able to grow. pQYxxxx represents the T2 generation seedlings transformed by flower dipping with various overexpression vectors and two lines are tested for each vector: The seminal roots of some transgenic Arabidopsis thaliana grow normally, and some do not, which indicates there are differences in 2,4-D resistance of the gene per se, or there may also be differences in expression quantity (different transformation events).

FIG. 5-2 shows a representative MCPA resistance testing of the T2 generation seedlings of the transgenic Arabidopsis thaliana transformed with an AAD-like gene. The plate medium comprises compound MCPA (0.1 μmol). Col-0: wild-type Arabidopsis thaliana Columbia-0 variety, whose seminal roots are not able to grow. pQYxxxx represents the T2 generation seedlings transformed by flower dipping with various overexpression vectors and two lines are tested for each vector: The seminal roots of some transgenic Arabidopsis thaliana grow normally, and some do not, which indicates there are differences in MCPA resistance of the gene per se, or there may also be differences in expression quantity (i.e. different transformation events).

FIG. 5-3 shows a representative 2,4-DB sodium resistance testing of the T2 generation seedlings of the transgenic Arabidopsis thaliana transformed with an AAD-like gene. The plate medium comprises compound 2,4-DB sodium (0.7 μmol). Col-0: wild-type Arabidopsis thaliana Columbia-0 variety, whose seminal roots are not able to grow. pQYxxxx represents the T2 generation seedlings transformed by flower dipping with various overexpression vectors and two lines are tested for each vector: The seminal roots of some transgenic Arabidopsis thaliana grow normally and some do not, which indicates there are differences in 2,4-DB sodium resistance of the gene per se, or there may also be differences in expression quantity (i.e. different transformation events).

FIG. 6-1 shows the resistance of the representative T2 generation seedlings of the transgenic Arabidopsis thaliana to 2,4-D (spray). Col-0: wild-type Arabidopsis thaliana Columbia-0 varieties all die after 10 days of spraying 2,4-D (100 g/ha), while all transgenic Arabidopsis thaliana lines show no apparent damage.

FIG. 6-2 shows the resistance of the representative T2 generation seedlings of the transgenic Arabidopsis thaliana to MCPA (spray). Col-0: wild-type Arabidopsis thaliana Columbia-0 varieties all die after 10 days of spraying MCPA (100 g/ha), while all transgenic Arabidopsis thaliana lines show no apparent damage.

FIG. 6-3 shows the resistance of the representative T2 generation seedlings of the transgenic Arabidopsis thaliana to 2,4-DB sodium (spray). Col-0: wild-type Arabidopsis thaliana Columbia-0 varieties are severely injured after 10 days of spraying 2,4-DB sodium (100 g/ha), while all transgenic Arabidopsis thaliana lines show no apparent damage.

FIG. 7-1 shows a pQY2329 maize overexpression vector inserted with the AAD-like gene QYD42 (SEQ ID NO. 83). The expression of QYD42 is regulated by the maize ubiquitin promoter and the Nos terminator; the expression of the screening gene BlpR (glufosinate resistance gene) is regulated by the CaMV35S promoter and its terminator.

FIG. 7-2 shows the resistance of the T1 generation seedlings of the representative transgenic maize event to 2,4-D (spray). WT (wild-type): the stem bases of maize B104 are swollen after 14 days of spraying 2,4-D (4.48 kg/ha), while the stem base of the pQY2329 transgenic event shows no apparent deformity.

FIG. 8-1 shows a pQY2330 soybean overexpression vector inserted with the AAD-like gene QYD42 (SEQ ID NO. 83). The expression of QYD42 is regulated by the double CaMV35S promoter and the Nos terminator; the expression of the screening gene BlpR (glufosinate resistance gene) is regulated by the MAS (mannopine synthase) promoter and its terminator.

FIG. 8-2 shows the resistance of the T1 generation seedlings of the representative transgenic soybean events to 2,4-D (spray). WT (wild-type): the soybeans William82 die after 10 days of spraying 2,4-D (4.48 kg/ha), while the pQY2330 transgenic event remains normal, and no differences show between the water spraying controls.

### DETAILED DESCRIPTION OF THE INVENTION

The following definitions and methods are provided to better define the invention and to guide those of ordinary skill in the art in the practice of the invention. Unless otherwise noted, terms are to be understood according to conventional usage by those of ordinary skill in the relevant art.

In the present invention, “hormone herbicide” refers to a substance that has herbicidal activity per se or a substance that is used in combination with other herbicides and/or additives which can change its effect, and belongs to plant hormone-disrupting herbicides. Plant hormone-disrupting herbicides are well known in the art, for example, comprising at least one of the following effective ingredients or derivatives thereof:
(1) Pyridine carboxylic acids: 4-amino-3,5,6-trichloropicolinic acid (picloram), 2-[(4-amino-3,5-dichloro-6-fluoro-2-pyridinyl)oxy]acetic acid (fluroxypyr), fluroxypyr-meptyl, aminopyralid, clopyralid, [(3,5,6-trichloro-2-pyridinyl)oxy]acetic acid (triclopyr), Florpyrauxifen-benzyl, Halauxifen-methyl, fluchloraminopyr-tefuryl, etc.;
(2) Benzoic acids: dicamba, chloramben, 2,3,6-TBA, naptalam, etc.;
(3) Phenoxycarboxylic acids: (2,4-dichlorophenoxy)acetic acid (2,4-D), 4-(2,4-dichlorophenoxy)butanoic acid (2,4-DB), 2,4-D isopropionic acid, clomeprop, (4-chloro-2-methylphenoxy)acetic acid (MCPA), mecoprop, 4-(4-chloro-2-methylphenoxy) butanoic acid (MCPB), etc.;
(4) Quinoline carboxylic acids: quinclorac, quinmerac, etc.;
(5) Others: benazolin-ethyl, etc.

“ACCase inhibitor herbicide” refers to a herbicide that targets acetyl-CoA carboxylase, which is well known in the art, for example, comprising at least one of the following effective ingredients or derivatives thereof:
(1) Aryloxy phenoxy propionates: quizalofop-P-ethyl, clodinafop-propargyl, cyhalofop-butyl, diclofop-methyl, fenoxaprop-P, fluazifop-P, haloxyfop-P, metamifop, propaquizafop, quizalofop-P-tefuryl, etc.;
(2) Cyclohexenones: alloxydim, clethodim, profoxydim, butroxydim, cycloxydim, sethoxydim, tepraloxydim, tralkoxydim, etc.;
(3) Benzo pyridines: pinoxaden, etc.

In the context of the present description, if an abbreviation of a generic name of active compound is used, it includes in each case all conventional derivatives thereof, such as esters and salts as well as isomers, in particular optical isomers, in particular one or more commercially available forms thereof. If the generic name denotes an ester or a salt, it also includes in each case all other conventional derivatives, such as other esters and salts, free acids and neutral compounds, as well as isomers, in particular optical isomers, in particular one or more commercially available forms thereof. The chemical name given to a compound means at least one compound encompassed by the generic name, and generally the preferred compound. For example, 2,4-D or 2,4-DB derivatives include, but are not limited to, salts of 2,4-D or 2,4-DB, such as sodium salt, potassium salt, dimethylammonium salt, triethanol ammonium salt, isopropylamine salt, choline salt, etc., and esters of 2,4-D or 2,4-DB, such as methyl ester, ethyl ester, butyl ester, isooctyl ester, etc.; MCPA derivatives include, but are not limited to, MCPA sodium salt, potassium salt, dimethylammonium salt, isopropylamine salt, etc., and MCPA methyl ester, ethyl ester, isooctyl ester, ethyl thioester and the like.

In the present invention, the terms “herbicide tolerance” and “herbicide resistance” are used interchangeably, both referring to herbicide tolerance and herbicide resistance, meaning a plant, seed, cell or plant part's ability to resist the toxic effects of one or more herbicide(s). The herbicide tolerance of a plant, seed, cell or plant part may be measured by comparing the plant, seed, cell or plant part to a suitable control. For example, the herbicide tolerance may be measured or assessed by applying an herbicide to a plant comprising a recombinant DNA molecule encoding a protein capable of conferring herbicide tolerance (the test plant) and a plant not comprising the recombinant DNA molecule encoding the protein capable of conferring herbicide tolerance (the control plant) and then comparing the plant injury of the two plants, where herbicide tolerance of the test plant is indicated by a reduced injury rate as compared to the injury rate of the control plant. An herbicide-tolerant plant, seed, cell or plant part exhibits a decreased response to the toxic effects of an herbicide when compared to a control plant, seed, cell or plant part. As used herein, an “herbicide tolerance trait” is a transgenic trait imparting improved herbicide tolerance to a plant as compared to a wild-type plant or control plant.

In the present invention, “wild-type” means a naturally occurring, which is relative to mutation.

The terms “protein”, “polypeptide” and “peptide” can be used interchangeably in the present invention and refer to a polymer of amino acid residues, including polymers of chemical analogs in which one or more amino acid residues are natural amino acid residues. The proteins and polypeptides of the present invention may be recombinantly produced or chemically synthesized.

As is well known in the art, one or more amino acid residues can be deleted from the N- and/or C-terminus of a protein, and the protein still retains the function and activity. In the present invention, “bioactive fragment” means a portion of the protein of the present invention which retains the biological activity of the protein. For example, a bioactive fragment of a protein may be a portion of the protein that lacks one or more (for example, 1-50, 1-25, 1-10 or 1-5, e.g., 1, 2, 3, 4 or 5) amino acid residues at the N- and/or C-terminus of the protein, but still retains the biological activity of the full-length protein.

In the present invention, the “stringent conditions” may refer to conditions of 6 M urea, 0.4% SDS and 0.5×SSC or hybridization conditions equivalent thereto, and may also refer to conditions with higher stringency, such as 6 M urea, 0.4% SDS, 0.1×SSC or hybridization conditions equivalent thereto. In various conditions, the temperature may be above about 40° C., for example, and if more stringent conditions are required, the temperature may be, such as, about 50° C., and further may be about 65° C.

It will be apparent for a person skilled in the art that a variety of different nucleic acid sequences can encode the amino acid sequences disclosed herein due to the degeneracy of genetic codes. It is within the ability of one of ordinary skill in the art to generate other nucleic acid sequences encoding a same protein, and thus the present invention encompasses nucleic acid sequences encoding the same amino acid sequence due to the degeneracy of genetic codes. For example, in order to achieve high expression of a heterologous gene in a target host organism, such as a plant, the gene can be optimized using host-preferred codons for better expression.

In the present invention, the “host organism” should be understood as any mono- or multicellular organism into which a nucleic acid encoding a mutant protein can be introduced, including, for example, bacteria such as Escherichia coli, fungi such as yeast (e.g., Saccharomyces cerevisiae), molds (e.g., Aspergillus), plant cells, plants and the like.

The terms “polynucleotide” and “nucleic acid” can be used interchangeably in the present invention and include DNA, RNA or RNA/DNA hybrid, which may be double-stranded or single-stranded. The term “isolated”, when referring to a nucleic acid, means a nucleic acid that is apart from a substantial portion of the genome in which it naturally occurs and/or is substantially separated from other cellular components which naturally accompany the nucleic acid. For example, any nucleic acid that has been produced synthetically (e.g., by serial base condensation) is considered to be isolated. Likewise, nucleic acids that are recombinantly expressed, cloned, produced by a primer extension reaction (e.g., PCR), or otherwise excised from a genome are also considered to be isolated.

The term “control” means an experimental control designed for comparison purposes. For example, a control plant in a transgenic plant analysis is a plant of the same type as the experimental plant (i.e., the plant to be tested) but does not contain the transgenic insert, recombinant DNA molecule, or DNA construct of the experimental plant.

The term “recombinant” refers to a non-natural DNA, polypeptide or protein that is the result of genetic engineering and as such would not normally be found in nature and is created by human intervention. A “recombinant DNA molecule” is a DNA molecule comprising a DNA sequence that does not naturally occur and as such is the result of human intervention, for example, a DNA molecule that encodes an engineered protein. Another example is a DNA molecule comprised of a combination of at least two DNA molecules heterologous to each other, such as a protein-coding DNA molecule and an operably linked heterologous promoter. A “recombinant polypeptide” or “recombinant protein” is a polypeptide or protein comprising an amino acid sequence that does not naturally occur and as such is the result of human intervention, for example, an engineered protein.

The term “transgene” refers to a DNA molecule artificially incorporated into the genome of an organism as a result of human intervention, such as by plant transformation methods. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. Transgenic plants of the present invention comprise the recombinant DNA molecules and the engineered proteins provided by the invention.

The term “heterologous” refers to the relationship between two or more things derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell or organism into which it is inserted when it would not naturally occur in that particular cell or organism.

The term “protein-coding DNA molecule” or “polypeptide-coding DNA molecule” refers to a DNA molecule comprising a nucleotide sequence that encodes a protein or polypeptide. A “protein-coding sequence” or “polypeptide-coding sequence” means a DNA sequence that encodes a protein or polypeptide. A “sequence” means a sequential arrangement of nucleotides or amino acids. The boundaries of a protein-coding sequence or polypeptide-coding sequence are usually determined by a translation start codon at the 5′-terminus and a translation stop codon at the 3′-terminus. A protein-coding molecule or polypeptide-coding molecule may comprise a DNA sequence encoding a protein or polypeptide sequence. As used herein, “transgene expression”, “expressing a transgene”, “protein expression”, “polypeptide expression”, “expressing a protein”, and “expressing a polypeptide” mean the production of a protein or polypeptide through the process of transcribing a DNA molecule into messenger RNA (mRNA) and translating the mRNA into polypeptide chains, which may be ultimately folded into proteins. A protein-coding DNA molecule or polypeptide-coding DNA molecule may be operably linked to a heterologous promoter in a DNA construct for use in expressing the protein or polypeptide in a cell transformed with the recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a protein-coding DNA molecule or polypeptide-coding DNA molecule in a DNA construct where the two DNA molecules are so arranged that the promoter may affect the expression of the transgene.

The term “DNA construct” is a recombinant DNA molecule comprising two or more heterologous DNA sequences. DNA constructs are useful for transgene expression and may be comprised in vectors and plasmids. DNA constructs may be used in vectors for the purpose of transformation, that is the introduction of heterologous DNA into a host cell, in order to produce transgenic plants and cells, and as such may also be contained in the plasmid DNA or genomic DNA of a transgenic plant, seed, cell or plant part. As used herein, a “vector” means any recombinant DNA molecule that may be used for the purpose of plant transformation. Recombinant DNA molecules as set forth in the sequence listing can, for example, be inserted into a vector as part of a construct having the recombinant DNA molecule operably linked to a promoter that functions in a plant to drive expression of the engineered protein encoded by the recombinant DNA molecule. Methods for constructing DNA constructs and vectors are well known in the art. The components for a DNA construct, or a vector comprising a DNA construct, generally include, but are not limited to, one or more of the following: a suitable promoter for the expression of an operably linked DNA, an operably linked non-human protein-coding DNA molecule, and a 3′ untranslated region (3′-UTR). Promoters useful in practicing the present invention include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional optional components include, but are not limited to, one or more of the following elements: 5′-UTR, enhancer, leader sequence, cis-acting element, intron, chloroplast transit peptides (CTP), and one or more selectable marker transgenes.

The DNA constructs of the invention may include a CTP molecule operably linked to the protein-coding DNA molecules provided by the invention. A CTP useful in practicing the present invention includes those that function to facilitate localization of the engineered protein molecule within the cell. By facilitating protein localization within the cell, the CTP may increase the accumulation of engineered protein, protect it from proteolytic degradation, enhance the level of herbicide tolerance, and thereby reduce levels of injury after herbicide application. CTP molecules used in the present invention are known in the art and include, but are not limited to the Arabidopsis thaliana EPSPS CTP (Klee et al., 1987), the Petunia hybrida EPSPS CTP (della-Cioppa et al., 1986), the maize cab-m7 signal sequence (Becker et al., 1992; PCT WO 97/41228) and the pea glutathione reductase signal sequence (Creissen et al., 1991; PCT WO 97/41228).

Recombinant DNA molecules of the present invention may be synthesized and modified by methods known in the art, either completely or in part, especially where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Wisconsin (Accelrys Inc., San Diego, CA), MEGAlign (DNAStar, Inc., 1228S. Park St., Madison, Wis.53715), and MUSCLE (version 3.6) (RCEdgar, Nucleic Acids Research (2004)32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

In the present invention, “plant” should be understood as any differentiated multicellular organism capable of performing photosynthesis, in particular monocotyledonous or dicotyledonous plants, for example, (1) food crops: Oryza spp., like Oryza sativa, Oryza latifolia, Oryza sativa, Oryza glaberrima; Triticum spp., like Triticum aestivum, T. Turgidum ssp. durum; Hordeum spp., like Hordeum vulgare, Hordeum arizonicum; Secale cereale; Avena spp., like Avena sativa, Avena fatua, Avena byzantine, Avena fatua var. sativa, Avena hybrida; Echinochloa spp., like Pennisetum glaucum, Sorghum, Sorghum bicolor, Sorghum vulgare, Triticale, Zea mays or Maize, Millet, Rice, Foxtail millet, Proso millet, Sorghum bicolor, Panicum, Fagopyrum spp., Panicum miliaceum, Setaria italica, Zizania palustris, Eragrostis tef, Panicum miliaceum, Eleusine coracana; (2) legume crops: Glycine spp. like Glycine max, Soja hispida, Soja max, Vicia spp., Vigna spp., Pisum spp., field bean, Lupinus spp., Vicia, Tamarindus indica, Lens culinaris, Lathyrus spp., Lablab, broad bean, mung bean, red bean, chickpea; (3) oil crops: Arachis hypogaea, Arachis spp, Sesamum spp., Helianthus spp. like Helianthus annuus, Elaeis like Eiaeis guineensis, Elaeis oleifera, soybean, Brassicanapus, Brassica oleracea, Sesamum orientale, Brassica juncea, Oilseed rape, Camellia oleifera, oil palm, olive, castor-oil plant, Brassica napus L., canola; (4) fiber crops: Agave sisalana, Gossypium spp. like Gossypium, Gossypium barbadense, Gossypium hirsutum, Hibiscus cannabinus, Agave sisalana, Musa textilis Nee, Linum usitatissimum, Corchorus capsularis L, Boehmeria nivea (L.), Cannabis sativa, Cannabis sativa; (5) fruit crops: Ziziphus spp., Cucumis spp., Passiflora edulis, Vitis spp., Vaccinium spp., Pyrus communis, Prunus spp., Psidium spp., Punica granatum, Malus spp., Citrullus lanatus, Citrus spp., Ficus carica, Fortunella spp., Fragaria spp., Crataegus spp., Diospyros spp., Eugenia unifora, Eriobotrya japonica, Dimocarpus longan, Carica papaya, Cocos spp., Averrhoa carambola, Actinidia spp., Prunus amygdalus, Musa spp. (Musa acuminate), Persea spp. (Persea Americana), Psidium guajava, Mammea Americana, Mangifera indica, Canarium album (Olea europaea), Caricapapaya, Cocos nucifera, Malpighia emarginata, Manilkara zapota, Ananas comosus, Annona spp., Citrus reticulate (Citrus spp.), Artocarpus spp., Litchi chinensis, Ribes spp., Rubus spp., pear, peach, apricot, plum, red bayberry, lemon, kumquat, durian, orange, strawberry, blueberry, hami melon, muskmelon, date palm, walnut tree, cherry tree; (6) rhizome crops: Manihot spp., Ipomoea batatas, Colocasia esculenta, tuber mustard, Allium cepa (onion), Eleocharis tuberose (water chestnut), Cyperus rotundus, Rhizoma dioscoreae; (7) vegetable crops: Spinacia spp., Phaseolus spp., Lactuca sativa, Momordica spp, Petroselinum crispum, Capsicum spp., Solanum spp. (such as Solanum tuberosum, Solanum integrifolium, Solanum lycopersicum), Lycopersicon spp. (such as Lycopersicon esculentum, Lycopersicon lycopersicum, Lycopersicon pyriforme), Macrotyloma spp., Kale, Luffa acutangula, lentil, okra, onion, potato, artichoke, asparagus, broccoli, Brussels sprouts, cabbage, carrot, cauliflower, celery, collard greens, squash, Benincasa hispida, Asparagus officinalis, Apium graveolens, Amaranthus spp., Allium spp., Abelmoschus spp., Cichorium endivia, Cucurbita spp., Coriandrum sativum, B. carinata, Rapbanus sativus, Brassica spp. (such as Brassica napus, Brassica rapa ssp., canola, oilseed rape, turnip rape, turnip rape, leaf mustard, cabbage, black mustard, canola (rapeseed), Brussels sprout, Solanaceae (eggplant), Capsicum annuum (sweet pepper), cucumber, luffa, Chinese cabbage, rape, cabbage, calabash, Chinese chives, lotus, lotus root, lettuce; (8) flower crops: Tropaeolum minus, Tropaeolum majus, Canna indica, Opuntia spp., Tagetes spp., Cymbidium (orchid), Crinum asiaticum L., Clivia, Hippeastrum rutilum, Rosa rugosa, Rosa Chinensis, Jasminum sambac, Tulipa gesneriana L., Cerasus sp., Pharbitis nil (L.) Choisy, Calendula officinalis L., Nelumbo sp., Bellis perennis L., Dianthus caryophyllus, Petunia hybrida, Tulipa gesneriana L., Lilium brownie, Prunus mume, Narcissus tazetta L., Jasminum nudiflorum LindL., Primula malacoides, Daphne odora, Camellia japonica, Michelia alba, Magnolia liliiflora, Viburnum macrocephalum, Clivia miniata, Malus spectabilis, Paeonia suffruticosa, Paeonia lactiflora, Syzygium aromaticum, Rhododendron simsii, Rhododendron hybridum, Michelia figo (Lour.) Spreng., Cercis chinensis, Kerria japonica, Weigela florida, Fructus forsythiae, Jasminum mesnyi, Parochetus communis, Cyclamen persicum MilL., Phalaenophsis hybrid, Dendrobium nobile, Hyacinthus orientalis, Iris tectorum Maxim, Zantedeschia aethiopica, Calendula officinalis, Hippeastrum rutilum, Begonia semperflorenshybr, Fuchsia hybrida, Begonia maculataRaddi, Geranium, Epipremnum aureum; (9) medicinal crops: Carthamus tinctorius, Mentha spp., Rheum rhabarbarum, Crocus sativus, Lycium chinense, Polygonatum odoratum, Polygonatum Kingianum, Anemarrhena asphodeloides Bunge, Radix ophiopogonis, Fritillaria cirrhosa, Curcuma aromatica, Amomum villosum Lour., Polygonum multiflorum, Rheum officinale, Glycyrrhiza uralensis Fisch, Astragalus membranaceus, Panax ginseng, Panax notoginseng, Acanthopanax gracilistylus, Angelica sinensis, Ligusticum wallichii, Bupleurum sinenses DC., Datura stramonium Linn., Datura metel L., Mentha haplocalyx, Leonurus sibiricus L., Agastache rugosus, Scutellaria baicalensis, Prunella vulgaris L., Pyrethrum carneum, Ginkgo biloba L., Cinchona ledgeriana, Hevea brasiliensis (wild), Medicago sativa Linn, Piper Nigrum L., Radix Isatidis, Atractylodes macrocephala Koidz; (10) raw material crops: Hevea brasiliensis, Ricinus communis, Vernicia fordii, Moms alba L., Hops Humulus lupulus, Betula, Alnus cremastogyne Burk., Rhus verniciflua stokes; (11) pasture crops: Agropyron spp., Trifolium spp., Miscanthus sinensis, Pennisetum sp., Phalaris arundinacea, Panicum virgatum, prairiegrasses, Indiangrass, Big bluestem grass, Phleum pratense, turf, cyperaceae (Kobresia pygmaea, Carex pediformis, Carex humilis), Medicago sativa Linn, Phleum pratense L., Medicago sativa, Melilotus suavcolen, Astragalus sinicus, Crotalaria juncea, Sesbania cannabina, Azolla imbircata, Eichhornia crassipes, Amorpha fruticosa, Lupinus micranthus, Trifolium, Astragalus adsurgens pall, Pistia stratiotes Linn, Alternanthera philoxeroides, Lolium; (12) sugar crops: Saccharum spp., Beta vulgaris; (13) beverage crops: Camellia sinensis, Camellia Sinensis, tea, Coffee (Coffea spp.), Theobroma cacao, Humulus lupulus Linn.; (14) lawn plants: Ammophila arenaria, Poa spp. (Poa pratensis (bluegrass)), Agrostis spp. (Agrostis matsumurae, Agrostis palustris), Lolium spp. (Lolium), Festuca spp. (Festuca ovina L.), Zoysia spp. (Zoysiajaponica), Cynodon spp. (Cynodon dactylon/bermudagrass), Stenotaphrum secunda tum (Stenotaphrum secundatum), Paspalum spp., Eremochloa ophiuroides (centipedegrass), Axonopus spp. (carpetweed), Bouteloua dactyloides (buffalograss), Bouteloua var. spp. (Bouteloua gracilis), Digitaria sanguinalis, Cyperusrotundus, Kyllingabrevifolia, Cyperusamuricus, Erigeron canadensis, Hydrocotylesibthorpioides, Kummerowiastriata, Euphorbia humifusa, Viola arvensis, Carex rigescens, Carex heterostachya, turf; (15) tree crops: Pinus spp., Salix spp., Acer spp., Hibiscus spp., Eucalyptus spp., Ginkgo biloba, Bambusa sp., Populus spp., Prosopis spp., Quercus spp., Phoenix spp., Fagus spp., Ceiba pentandra, Cinnamomum spp., Corchorus spp., Phragmites australis, Physalis spp., Desmodium spp., Populus, Hedera helix, Populus tomentosa Carr, Viburnum odoratissinum, Ginkgo biloba L., Quercus, Ailanthus altissima, Schima superba, ilex purpurea, Platanus acerifolia, ligustrum lucidum, Buxus megistophylla LevL., Dahurian larch, Acacia mearnsii, Pinus massoniana, Pinus khasys, Pinus yunnanensis, Pinus finlaysoniana, Pinus tabuliformis, Pinus koraiensis, Juglans nigra, Citrus limon, Platanus acerifolia, Syzygium jambos, Davidia involucrate, Bombax malabarica L., Ceiba pentandra (L.), Bauhinia blakeana, Albizia saman, Albizzia julibrissin, Erythrina corallodendron, Erythrina indica, Magnolia gradiflora, Cycas revolute, Lagerstroemia indica, coniferous, macrophanerophytes, Frutex; (16) nut crops: Bertholletia excelsea, Castanea spp., Corylus spp., Carya spp., Juglans spp., Pistacia vera, Anacardium occidentale, Macadamia (Macadamia integrifolia), Carya illinoensis Koch, Macadamia, Pistachio, Badam, other plants that produce nuts; (17) others: Arabidopsis thaliana, Bra chiaria eruciformis, Cenchrus echinatus, Setaria faberi, Eleusine indica, Cadaba farinose, algae, Carex elata, ornamental plants, Carissa macrocarpa, Cynara spp., Daucus carota, Dioscorea spp., Erianthus sp., Festuca arundinacea, Hemerocallis fulva, Lotus spp., Luzula sylvatica, Medicago sativa, Melilotus spp., Moms nigra, Nicotiana spp., Olea spp., Ornithopus spp., Pastinaca sativa, Sambucus spp., Sinapis sp., Syzygium spp., Tripsacum dactyloides, Triticosecale rimpaui, Viola odorata, and the like.

In the present invention, the term “plant tissue” or “plant part” includes a plant cell, a protoplast, a plant tissue culture, a plant callus, a plant piece, a plant embryo, a pollen, an ovule, a leaf, a stem, a flower, a branch, a seedling, a fruit, a nucleus, a spike, a root, a root tip, an anther, and the like.

In the present invention, “plant cell” should be understood as any cell derived or found in a plant, which is capable of forming, for example, undifferentiated tissues such as calli, differentiated tissues such as embryos, constituent parts of a plant, plants, or seeds.

The transgenic plants, progeny, seeds, plant cells, and plant parts of the invention may also contain one or more additional transgenic traits. Additional transgenic traits may be introduced by crossing a plant containing a transgene comprising the recombinant DNA molecules provided by the invention with another plant containing an additional transgenic trait(s). As used herein, “crossing” means breeding two individual plants to produce a progeny plant. Two transgenic plants may thus be crossed to produce a progeny that contains the transgenic traits. As used herein, “progeny” means the offspring of any generation of a parent plant, and transgenic progeny comprises a DNA construct provided by the invention and inherited from at least one parent plant. Alternatively, additional transgenic trait(s) may be introduced by co-transforming a DNA construct for that additional transgenic trait(s) with a DNA construct comprising the recombinant DNA molecules provided by the invention (for example, with all the DNA constructs present as part of the same vector used for plant transformation) or by inserting the additional trait(s) into a transgenic plant comprising a DNA construct provided by the invention or vice versa (for example, by using any of the methods of plant transformation on a transgenic plant or plant cell). Such additional transgenic traits include, but are not limited to, increased insect resistance, increased water use efficiency, increased yield performance, increased drought resistance, increased seed quality, improved nutritional quality, hybrid seed production, and herbicide tolerance, in which the trait is measured with respect to a wild-type plant or control plant. Such additional transgenic traits are known to one skilled in the art; for example, a list of such traits is provided by Animal and Plant Health Inspection Service (APHIS), the United States Department of Agriculture (USDA) and can be found on their website at www.aphis.usda.gov.

Transgenic plants and progeny that contain a transgenic trait provided by the invention may be used with any breeding methods that are commonly known in the art. In plant lines comprising two or more transgenic traits, the transgenic traits may be independently segregating, linked, or a combination of both in plant lines comprising three or more transgenic traits. Back-crossing to a parental plant and out-crossing with a non-transgenic plant are also contemplated, as is vegetative propagation. Descriptions of breeding methods that are commonly used for different traits and crops are well known to those of skill in the art. To confirm the presence of the transgene(s) in a particular plant or seed, a variety of assays may be performed. Such assays include, for example, molecular biology assays, such as Southern and Northern blotting, PCR, and DNA sequencing; biochemical assays, such as detecting the presence of a protein product, for example, by immunological means (ELISAs and Western blotting) or by enzymatic function; plant part assays, such as leaf or root assays; and also, by analyzing the phenotype of a whole plant.

Introgression of a transgenic trait into a plant genotype is achieved as the result of the process of backcross conversion. A plant genotype into which a transgenic trait has been introgressed may be referred to as a backcross converted genotype, line, inbred, or hybrid. Similarly, a plant genotype lacking the desired transgenic trait may be referred to as an unconverted genotype, line, inbred, or hybrid.

As used herein, the term “comprising” means “including but not limited to”.

Unless specifically stated or implied, as used herein, the terms “a”, “an/an”, and “the” mean “at least one”. All patents, patent applications, and publications mentioned or cited herein are incorporated herein by reference in their entirety, with the same degree of citation as if they were individually cited.

The present invention is further described in detail below with reference to the specific embodiments, and the given examples are only to illustrate the present invention, not to limit the scope of the present invention. The experimental methods in the following examples are conventional methods unless otherwise specified. The materials, reagents, instruments and so on used in the following examples are commercially available unless otherwise specified.

### Example 1

Sources, sequences and synthesis of candidate genes for degrading hormone herbicides

1. Sources of Candidate Genes

The tfdA gene family (α-ketoglutaric acid-dependent taurine dioxygenase) derived from Ralstonia eutropha degrades 2,4-D through the contained TauD (Taurine catabolism dioxygenase) domain. So far, the gene family Database (Pfam Database) has 14,425 genes containing TauD domains, constituting 223 different organizational structures, which are vast resources of candidate genes. And 76 candidate genes were selected from the Pfam Database and other published literature for testing.

2. Sequences of Candidate Genes

The amino acid sequences of the 76 candidate enzymes for hormone herbicide degradation were SEQ ID NO. 1, SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31, SEQ ID NO. 33, SEQ ID NO. 35, SEQ ID NO. 37, SEQ ID NO. 39, SEQ ID NO. 41, SEQ ID NO. 43, SEQ ID NO. 45, SEQ ID NO. 47, SEQ ID NO. 49, SEQ ID NO. 51, SEQ ID NO. 53, SEQ ID NO. 55, SEQ ID NO. 57, SEQ ID NO. 59, SEQ ID NO. 61, SEQ ID NO. 63, SEQ ID NO. 65, SEQ ID NO. 67, SEQ ID NO. 69, SEQ ID NO. 71, SEQ ID NO. 73, SEQ ID NO. 75, SEQ ID NO. 77, SEQ ID NO. 79, SEQ ID NO. 81, SEQ ID NO. 83, SEQ ID NO. 85, SEQ ID NO. 87, SEQ ID NO. 89, SEQ ID NO. 91, SEQ ID NO. 93, SEQ ID NO. 95, SEQ ID NO. 97, SEQ ID NO. 99, SEQ ID NO. 101, SEQ ID NO. 103, SEQ ID NO. 105, SEQ ID NO. 107, SEQ ID NO. 109, SEQ ID NO. 111, SEQ ID NO. 113, SEQ ID NO. 115, SEQ ID NO. 117, SEQ ID NO. 119, SEQ ID NO. 121, SEQ ID NO. 123, SEQ ID NO. 125, SEQ ID NO. 127, SEQ ID NO. 129, SEQ ID NO. 131, SEQ ID NO. 133, SEQ ID NO. 135, SEQ ID NO. 137, SEQ ID NO. 139, SEQ ID NO. 141, SEQ ID NO. 143, SEQ ID NO. 145, SEQ ID NO. 147, SEQ ID NO. 149, SEQ ID NO. 151.

The encoding gene sequences thereof after plant codon preference optimization were SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, SEQ ID NO. 40, SEQ ID NO. 42, SEQ ID NO. 44, SEQ ID NO. 46, SEQ ID NO. 48, SEQ ID NO. 50, SEQ ID NO. 52, SEQ ID NO. 54, SEQ ID NO. 56, SEQ ID NO. 58, SEQ ID NO. 60, SEQ ID NO. 62, SEQ ID NO. 64, SEQ ID NO. 66, SEQ ID NO. 68, SEQ ID NO. 70, SEQ ID NO. 72, SEQ ID NO. 74, SEQ ID NO. 76, SEQ ID NO. 78, SEQ ID NO. 80, SEQ ID NO. 82, SEQ ID NO. 84, SEQ ID NO. 86, SEQ ID NO. 88, SEQ ID NO. 90, SEQ ID NO. 92, SEQ ID NO. 94, SEQ ID NO. 96, SEQ ID NO. 98, SEQ ID NO. 100, SEQ ID NO. 102, SEQ ID NO. 104, SEQ ID NO. 106, SEQ ID NO. 108, SEQ ID NO. 110, SEQ ID NO. 112, SEQ ID NO. 114, SEQ ID NO. 116, SEQ ID NO. 118, SEQ ID NO. 120, SEQ ID NO. 122, SEQ ID NO. 124, SEQ ID NO. 126, SEQ ID NO. 128, SEQ ID NO. 130, SEQ ID NO. 132, SEQ ID NO. 134, SEQ ID NO. 136, SEQ ID NO. 138, SEQ ID NO. 140, SEQ ID NO. 142, SEQ ID NO. 144, SEQ ID NO. 146, SEQ ID NO. 148, SEQ ID NO. 150, SEQ ID NO. 152. 3. Synthesis of candidate genes

It was planned to use pET15b as the backbone vector for expression in E. coli followed by screening. Clone was performed using infusion technology after enzyme digestion and ring opening at the pET15b NdeI/BamH1 site. Therefore, 5′-GCCGCGCGGCAGCCAT-3′ and 5′-TGTTAGCAGCCGGATCC-3′ linker sequences were respectively added to the 5′-termini and 3′-termini of the above-mentioned candidate gene sequences and sent to GenScript (Nanjing) Co., Ltd. for synthesis.

### Example 2

Construction of Overexpression, Purification and Chromogenic Reaction System of AAD12 Gene in Escherichia coli

AAD12 is a degradation gene for hormone herbicides discovered by Dow AgroSciences LLC (the U.S.), and has been transformed into maize, soybean and other crops for commercial applications along with hormone herbicides (T. R. Wright et al., Robust crop resistance to broadleaf and grass herbicides provided by aryloxyalkanoate dioxygenase transgenes. Proc. Natl. Acad. Sci. U.S.A. 107, 20240-20245 (2010)). AAD12 is capable of degrading 2,4-D and 5-chiral pyridinecarboxylic acid herbicides to produce 2,4-dichlorophenol and the like, which appear red. A chromogenic reaction system was established with AAD12 as the model gene to create a screening tool for large-scale screening of hormone herbicide genes.

1. Vector Construction

AAD12 has a full length of 293 amino acids, with a molecular weight of 31.7 kDa and an isoelectric point of 6.45. Its sequence was from the patent (U.S. Ser. No. 10/167,483-0004) published by Dow AgroSciences LLC. After plant codon optimization, 5′-GCCGCGCGGCAGCCAT-3′ and 5′-TGTTAGCAGCCGGATCC-3′ linker sequences were added to the 5′-terminus and 3′-terminus of AAD12 and sent to GenScript (Nanjing) Co., Ltd. for synthesis. The synthetic DNA was cloned into pET15b backbone at the NdeI/BamH1 site using the InFusion cloning technique to form an E. coli expression vector pET15b-AAD12.

2. Protein Expression and Purification

As reported in literature (Fukomori and Flausinger, Purification and characterization of 2,4-dichlorophenoxyacetate/alpha-ketoglutarate dioxygenase Journal of Biological Chemistry (1993) 268(32): 24311-24317), the constructed expression vector pET15b-AAD12 was transformed into E. coli BL21(DE3) and induced by IPTG for expression. After cells were collected and lysed, Ni-NTA column was used for purification and the purity of the protein was analyzed by SDS-PAGE.

3. Enzyme Activity Assay

Since phenolic products were produced by AAD-12-substrate catalytic reaction, 4-aminoantipyrine and potassium ferricyanide could be used to develop color with the products in an alkaline environment to generate red products with absorption at 510 nm, and the relevant parameters were directly read with a microplate reader.

4. Construction of a Chromogenic Screening System Using 2,4-D and Other Hormone Herbicides as Substrates

On the basis of testing the AAD12 protein activity with 2,4-D as the substrate, the conversion method of 2,4-D in bacteria was further tested to construct a high-throughput chromogenic reaction screening system for screening genes with degradation activities on 2,4-D and other hormone herbicides.

To transform the plasmids to be tested, an AAD12-expressing plasmid and a blank control plasmid pET15b were respectively transformed into the protein expression E. coli BL21(DE3) strains. A monoclone was selected and inoculated in a 10 ml centrifuge tube with 3 ml LB medium, and cultivated at 37° C., 200 rpm for 6-8 hours. The culture medium was added with 0.1 mM IPTG and 2 mM α-ketoglutaric acid, and then 0.5 mM 2,4-D. The temperature was switched to 28° C. and the cultivation continued overnight. In the next morning, 200 μL of each bacteria solution was taken and centrifuged to precipitate thallus, 180 μL of the supernatant was taken and added in a 96-well plate, and 20 μL of borate buffer of pH 10 was added. Then the mixture was added with 2 μL of 8% potassium ferricyanide and mixed well, followed by adding 2 μL of 2% 4-aminoantipyrine, and placed at room temperature for 5 min to observe the color and detect the A510 value.

The result of enzyme activity assay showed that compared to the control group with blank vectors, the reaction solution of the experimental group with AAD12 expression turned red when 2,4-D was used. It indicated that phenolic products were generated in the reaction solution of the experimental group, and the AAD12 expressed by E. coli had catalytic activity. Using a microplate reader to measure A510, it could also be detected that the absorption values of the experimental group were significantly higher than those of the control group.

In addition to 2,4-D, tests were also performed on another phenoxyacetic acid herbicide, MCPA, and 2,4-D isooctyl ester and 2,4-DB, and the results showed that the expressed AAD-12 enzyme had degradation activities on all these substrates, as shown in Table 1. The contents of herbicide substrates in each experimental and blank control group were measured by HPLC, and it turned out that the content of each substrate was significantly reduced after adding enzymes to catalyze reactions, proving each substrate was degraded.

TABLE 1
Determination of degradation activities of ADD-12 on phenoxy
acetic acid herbicides by spectrophotometry

| | | | | A510 of reaction group | | | |
|---|---|---|---|---|---|---|---|
| | A510 of blank group | | | Reaction | Reaction | Reaction | Increased |
| Substrate | Blank 1 | Blank 2 | Blank 3 | 1 | 2 | 3 | A510 |
| 2,4-D | 0.1548 | 0.1513 | 0.1467 | 0.2865 | 0.2766 | 0.2652 | +0.130 |
| 2,4-D | | | | | | | |
| isooctyl | 0.1318 | 0.1369 | 0.1384 | 0.1570 | 0.1578 | 0.1497 | +0.019 |
| ester | | | | | | | |
| 2,4-DB | 0.1208 | N/A | 0.1152 | 0.1373 | 0.1326 | 0.1345 | +0.017 |
| MCPA | 0.1722 | 0.1659 | N/A | 0.1998 | 0.2076 | N/A | +0.035 |

The bacteria solution also appeared red when 2,4-D was directly added into the medium of AAD12-expressing bacteria, as shown in FIG. 1. It indicated that the culture method of adding substrates into medium could be used to realize degradation and color development in bacteria, so as to determine whether a candidate gene product degrades 2,4-D. The chromogenic screening system for degradation in bacteria can improve measurement throughput and screen multiple AAD gene-expressing bacteria rapidly.

### Example 3

Screening of AAD Genes Using the Constructed Chromogenic Screening System for Degradation in Bacteria

The 76 ADD genes were screened using the chromogenic reaction screening method of AAD12 converting 2,4-D into phenols in bacteria. 2,4-D was added into these bacteria solutions of AAD gene-expressing recombinant E. coli and cultivation was continued for color development. And 44 recombinant E. coli solutions were detected to appear red, indicating the AAD-like enzymes encoded by the 44 genes had catalytic activities on 2,4-D (see Table 2).

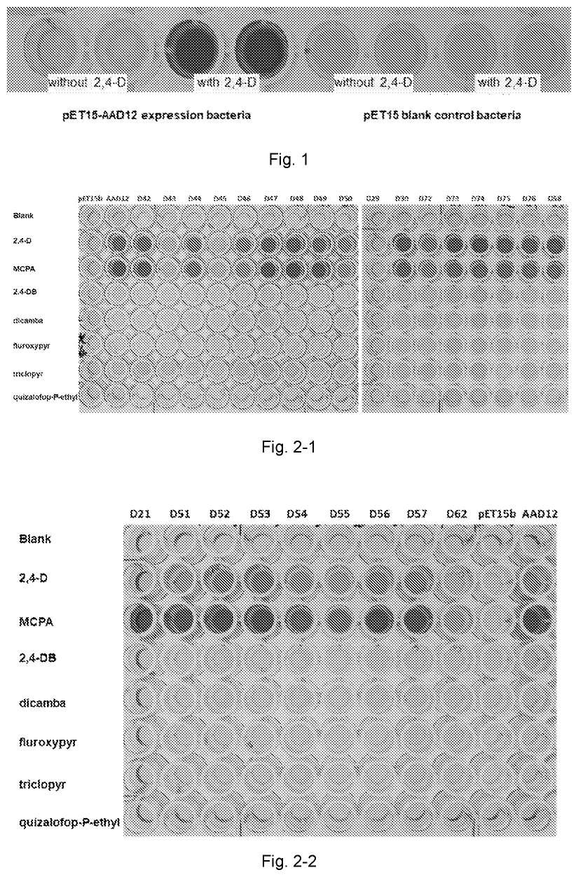

And 30 of the AAD gene-expressing bacteria (see FIG. 2-1 for No. D42, D43, D44, D45, D46, D47, D48, D49, D50, D30, D72, D73, D74, D75, D76 and D58; see FIG. 2-2 for No. D21, D51, D52, D53, D54, D55, D56, D57, D62, D=QYD) that could degrade 2,4-D were further tested for degrading other hormone herbicide compounds (MCPA, 2,4-DB, dicamba, fluroxypyr, triclopyr) and quizalofop-P-ethyl which belongs to ACCase inhibitor herbicide in bacteria. In addition, D26, D32, D33, D34 and D35 also appeared red reactions. The results showed that the enzymes also degraded MCPA in addition to 2,4-D; among which D42, D43, D46, D47, D48, D30, D72, D73, D74, D76 and D58 (D=QYD) showed slight degradation to triclopyr; did not degrade the others.

TABLE 2
Summary of identification for hormone herbicide resistance of 76 AAD-like genes

| | | | % | | | | | | | |
|---|---|---|---|---|---|---|---|---|---|---|
| | | | sequence | | | | | | | |
| | | | identity | | | | | | | |
| | | | aligned | | | | | | | |
| | | | with the | | | | | | | |
| | | | most | | | | | | Resistance of | |
| | | No. of the most | similar | | Chromogenic reaction of | | | Expression | transgenic | |
| SEQ | Test | similar amino | sequence | | degrading herbicides in bacteria | | | vector No. in | Arabidopsis | |
| ID | material | acid sequence | in GQ | Novelty | | | Tri- | Arabidopsis | thaliana | |
| NO | No. | in GQ search | search | of use | 2,4-D | MCPA | clopyr | thaliana | 2,4-D | MCPA |
| 1 | QYD1 | US20170022515- | 100.00 | NO | Yes | NT | NT | pQY759 | Yes | NT |
| | | 0011 (AAD1) | | | | | | | | |
| 3 | QYD2 | US20170009214- | 100.00 | NO | Yes | NT | NT | pQY760 | Yes | NT |
| | | 0019 (AAD2) | | | | | | | | |
| 5 | QYD3 | US20160002662- | 100.00 | NO | Yes | NT | NT | PQY761 | Yes | NT |
| | | 0002 (AAD13) | | | | | | | | |
| 7 | QYD4 | US20100251432-0002 | 99.65 | NO | Yes | NT | NT | pQY762 | Yes | NT |
| | | (AAD13 mutant) | | | | | | | | |
| 9 | QYD5 | NT | NT | NT | NO | NT | NT | PQY763 | Yes | NT |
| 11 | QYD6 | NT | NT | NT | NO | NT | NT | PQY764 | NO | NT |
| 13 | QYD7 | NT | NT | NT | NO | NT | NT | pQY765 | NO | NT |
| 15 | QYD8 | NT | NT | NT | NO | NT | NT | pQY766 | NO | NT |
| 17 | QYD9 | NT | NT | NT | NO | NT | NT | pQY767 | NO | NT |
| 19 | QYD10 | NT | NT | NT | NO | NT | NT | PQY768 | NO | NT |
| 21 | QYD11 | NT | NT | NT | NO | NT | NT | pQY769 | NO | NT |
| 23 | QYD12 | NT | NT | NT | NO | NT | NT | PQY770 | NO | NT |
| 25 | QYD13 | NT | NT | NT | NO | NT | NT | pQY771 | NO | NT |
| 27 | QYD14 | NT | NT | NT | NO | NT | NT | PQY772 | Yes | NT |
| 29 | QYD15 | NT | NT | NT | NO | NT | NT | PQY773 | NO | NT |
| 31 | QYD16 | NT | NT | NT | NO | NT | NT | PQY774 | NO | NT |
| 33 | QYD17 | NT | NT | NT | NO | NT | NT | PQY775 | NO | NT |
| 35 | QYD18 | NT | NT | NT | NO | NT | NT | PQY776 | NO | NT |
| 37 | QYD19 | NT | NT | NT | NO | NT | NT | PQY777 | NO | NT |
| 39 | QYD20 | NT | NT | NT | NO | NT | NT | PQY778 | NO | NT |
| 41 | QYD21 | US20070042383-1597 | 39.79 | Yes | Yes | Yes | NO | PQY779 | Yes | NT |
| 43 | QYD22 | NT | NT | NT | NO | NT | NT | pQY780 | NO | NT |
| 45 | QYD23 | NT | NT | NT | NO | NT | NT | pQY781 | NO | NT |
| 47 | QYD24 | NT | NT | NT | NO | NT | NT | pQY782 | NO | NT |
| 49 | QYD25 | NT | NT | NT | NO | NT | NT | pQY783 | NO | NT |
| 51 | QYD26 | US20190017066-0022 | 38.81 | Yes | Yes | Yes | NO | pQY784 | Yes | NT |
| 53 | QYD27 | NT | NT | NT | NO | NT | NT | pQY785 | NO | NT |
| 55 | QYD28 | NT | NT | NT | NO | NT | NT | pQY786 | NO | NT |
| 57 | QYD29 | NT | NT | NT | NO | NT | NT | PQY795 | NO | NT |
| 59 | QYD30 | WO2019023192-0277 | 65.00 | Yes | Yes | Yes | Yes | pQY796 | Yes | NT |
| 61 | QYD31 | NT | NT | NT | NO | NT | NT | PQY797 | Yes | NT |
| 63 | QYD32 | US20160002662-0002 | 68.68 | Yes | Yes | NO | NO | pQY798 | Yes | NT |
| 65 | QYD33 | US20160002662-0002 | 61.19 | Yes | Yes | NO | NO | pQY799 | NO | NT |
| 67 | QYD34 | US20160002662-0002 | 63.70 | Yes | Yes | NO | NO | pQY2301 | Yes | NT |
| 69 | QYD35 | US20160002662-0002 | 75.79 | Yes | Yes | NO | NO | pQY2302 | NO | NT |
| 71 | QYD36 | NT | NT | NT | NO | NT | NT | pQY2303 | NO | NT |
| 73 | QYD37 | NT | NT | NT | NO | NT | NT | pQY2304 | NO | NT |
| 75 | QYD38 | NT | NT | NT | NO | NT | NT | pQY2305 | NO | NT |
| 77 | QYD39 | NT | NT | NT | NO | NT | NT | pQY2306 | NO | NT |
| 79 | QYD40 | NT | NT | NT | NO | NT | NT | pQY2307 | NO | NT |
| 81 | QYD41 | NT | NT | NT | NO | NT | NT | pQY2308 | NO | NT |
| 83 | QYD42 | EP3241430-0002 | 87.89 | Yes | Yes | Yes | Yes | pQY2309 | Yes | Yes |
| 85 | QYD43 | US20170009214-0002 | 80.70 | Yes | Yes | Yes | Yes | pQY2310 | Yes | Yes |
| 87 | QYD44 | US20160002662-0002 | 94.06 | Yes | Yes | Yes | NO | pQY2311 | Yes | Yes |
| 89 | QYD45 | US20160002662-0004 | 88.54 | Yes | Yes | Yes | NO | pQY2312 | Yes | Yes |
| 91 | QYD46 | US20160002662-0002 | 86.41 | Yes | Yes | Yes | Yes | pQY2313 | Yes | Yes |
| 93 | QYD47 | US20160002662-0002 | 79.37 | Yes | Yes | Yes | Yes | pQY2314 | Yes | Yes |
| 95 | QYD48 | US20160002662-0002 | 80.77 | Yes | Yes | Yes | Yes | pQY2315 | Yes | Yes |
| 97 | QYD49 | US20160002662-0002 | 81.47 | Yes | Yes | Yes | NO | pQY2316 | Yes | Yes |
| 99 | QYD50 | US20160002662-0002 | 80.77 | Yes | Yes | Yes | NO | pQY2317 | Yes | Yes |
| 101 | QYD51 | US20160002662-0002 | 78.52 | Yes | Yes | Yes | NO | pQY2318 | Yes | Yes |
| 103 | QYD52 | US20160002662-0002 | 78.32 | Yes | Yes | Yes | NO | pQY2319 | Yes | NO |
| 105 | QYD53 | US20160002662-0002 | 75.35 | Yes | Yes | Yes | NO | pQY2320 | Yes | Yes |
| 107 | QYD54 | US20160002662-0002 | 75.09 | Yes | Yes | Yes | NO | pQY2321 | NT | NT |
| 109 | QYD55 | US20160002662-0002 | 76.66 | Yes | Yes | Yes | NO | pQY2322 | Yes | NT |
| 111 | QYD56 | US20160002662-0002 | 75.44 | Yes | NO | Yes | NO | pQY2323 | Yes | NO |
| 113 | QYD57 | US20160002662-0002 | 75.44 | Yes | Yes | Yes | NO | pQY2324 | Yes | Yes |

| | | | | | | | |
|---|---|---|---|---|---|---|---|
| 115 | QYD58 | US20160002662-0002 | 81.47 | Yes | Yes | Yes | Yes |
| 117 | QYD59 | NT | NT | NT | NO | NT | NT |
| 119 | QYD60 | US20160002662-0002 | 100.00 | NO | Yes | NT | NT |
| 121 | QYD61 | US20190024109-0292 | 100.00 | NO | Yes | NT | NT |
| 123 | QYD62 | US20140141044-13793 | 100.00 | Yes | Yes | Yes | NO |
| | | (for medical field) | | | | | |
| 125 | QYD63 | US20140179534-0002 | 100.00 | NO | Yes | NT | NT |
| 127 | QYD64 | US20180066275-0002 | 100.00 | NO | Yes | NT | NT |
| 129 | QYD65 | CN103013939-0002 | 100.00 | NO | Yes | NT | NT |
| 131 | QYD66 | US20140194292-0002 | 100.00 | NO | Yes | NT | NT |
| 133 | QYD67 | US20180244732-0002 | 100.00 | NO | Yes | NT | NT |
| 135 | QYD68 | JP2018506305-0002 | 100.00 | NO | Yes | NT | NT |
| 137 | QYD69 | US20170211087-0004 | 100.00 | NO | Yes | NT | NT |
| 139 | QYD70 | US20190017066-0048 | 100.00 | NO | Yes | NT | NT |
| 141 | QYD71 | CN109456986-0002 | 100.00 | NO | Yes | NT | NT |
| 143 | QYD72 | US20160002662-0002 | 65.14 | No | Yes | Yes | Yes |
| 145 | QYD73 | US20160002662-0002 | 75.79 | Yes | Yes | Yes | Yes |
| 147 | QYD74 | US20160002662-0002 | 72.38 | Yes | Yes | Yes | Yes |
| 149 | QYD75 | US20160002662-0002 | 75.65 | Yes | Yes | Yes | NO |
| 151 | QYD76 | US20160002662-0002 | 75.09 | Yes | Yes | Yes | Yes |

Note:
1. NT represented not tested;
2. Genes marked with background colors represented the value of its herbicide resistance was discovered in this study for the first time;
3. GQ represented GenomeQuest

### Example 4

Creation and Resistance Testing of a Transgenic Arabidopsis thaliana Transformed with ADD Genes

1. Construction of a Transgenic Arabidopsis thaliana Overexpression Vector

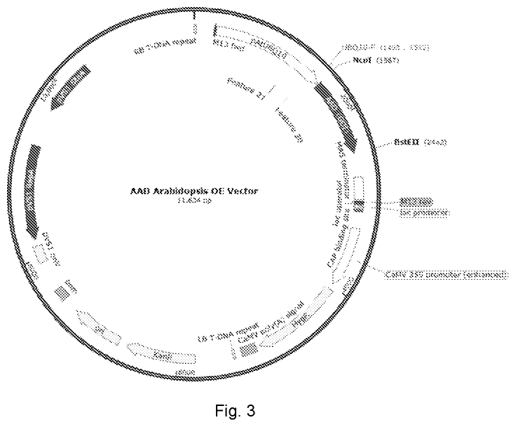

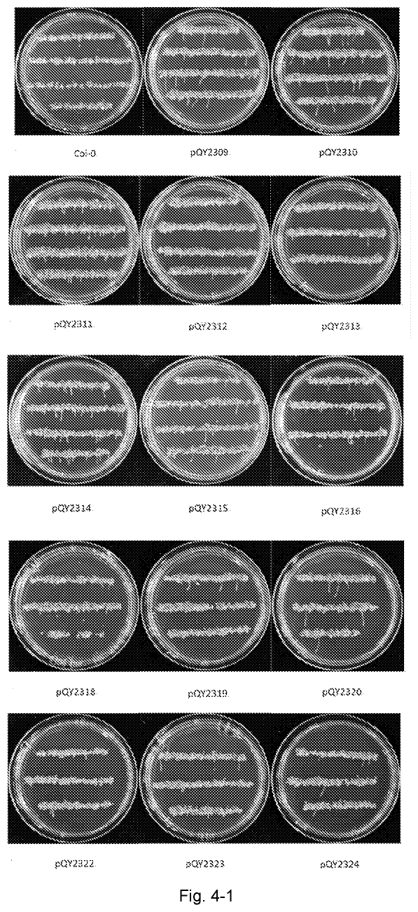

As shown in FIG. 3, using pQY259 as the backbone vector and NcoI/BstEII as the cloning site, these AAD genes that can degrade 2,4-D in bacteria overexpressed in E. coli were cloned into the backbone vector pQY259 to produce a series of Arabidopsis thaliana overexpression vectors. Expression in Arabidopsis thaliana was regulated by its AtUbi10 Arabidopsis thaliana ubiquitin promoter and the MAS (mannopine synthase) terminator. Hygromycin resistance gene (HygR=hpt) served as the selection marker.

2. Genetic Transformation of Arabidopsis thaliana

Preparation of Agrobacterium infection solution: An activated Agrobacterium (GV3101) monoclone was picked and inoculated in 30 ml YEP liquid medium [yeast powder 10 g/L, tryptone 10 g/L, NaCl 5 g/L, 25 mg/L Rifampicin and 50 mg/L Kanamycin], shaking-cultured at 200 rmp/min, 28° C. overnight until the OD600 was about 1.0-1.5, then centrifuged at 6000 rmp/min for 10 min to collect the bacteria, and the supernatant was discarded. Arabidopsis thaliana infection solution (sucrose 50 g/L, Silwet L-77 300 μL/L, no need to adjust pH) was used to resuspend the cells to OD600=0.8 for later use.

Genetic transformation of Arabidopsis thaliana by flower dipping: Before transformation of Arabidopsis thaliana plants, attentions were paid to whether the plants were growing well, the inflorescences were abundant, and no stress response occurred. The first transformation was performed when plants were about 20 cm high. Plants could be properly watered if the soil was dry. The grown siliques were cut off with scissors the day before transformation. The inflorescences of the plants to be transformed were soaked in the above-mentioned Agrobacterium solution for 30 seconds to 1 minute, which was stirred gently during the period. And a layer of liquid film should appear on the infiltrated plant. The transformed plants were cultured in a dark environment for 24 hours, and then taken out and grown in a normal light environment. A second transformation could be performed in the same way a week later.

The seeds could be harvested when becoming mature. After harvesting, the seeds were dried at 30° C. in an oven for about a week.

3. Screening of T1 Generation Transgenic Plants and Drug-Resistant Plants

Seeds were treated with disinfectant for 5 min, washed with sterile water for 5 times and then one part was spread on MS transgenic screening medium (containing 30 μg/mL hygromycin, 100 μg/mL carbenicillin) to screen transgenic seedlings, and the other part was spread on MS medicated medium [containing 250 μg/mL Cefotaxime sodium and 0.3 μM 2,4-D] to screen herbicide-resistant seedlings at mean time. The petri dish spread with seeds was placed at 4° C. for vernalization for 3 days, and taken out to a illumination incubator for cultivation (temperature of 22° C., 16 hours of light, 8 hours of darkness, light intensity of 100-150 μmol/m²/s, humidity of 75%). After one week, it was observed that the roots of the wild-type control seedlings were not elongated and their growth was inhibited, while the transgenic seedlings had normal root growth, showing drug resistance. The transgenic positive seedlings and herbicide-resistant seedlings were photographed and transplanted into small pots filled with soil for cultivation.

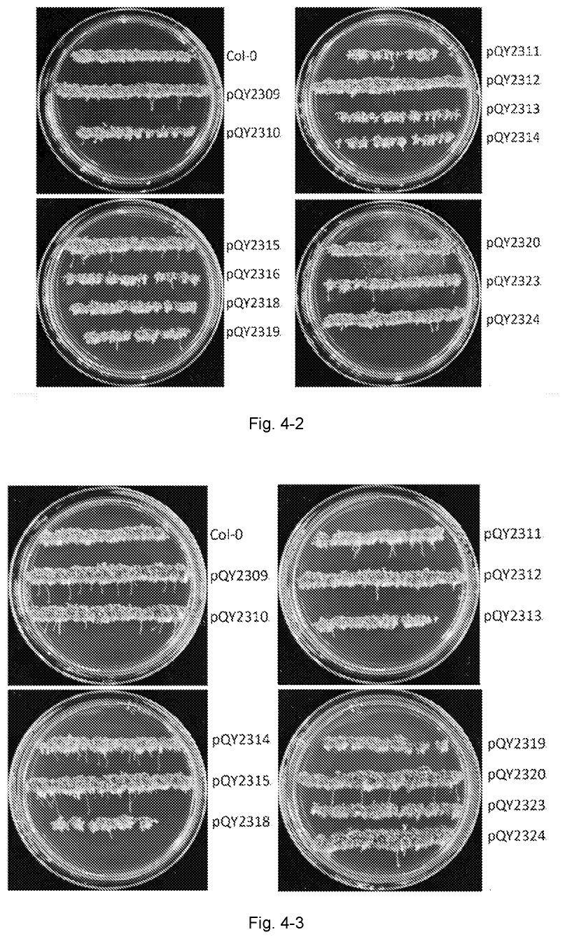

The results (FIG. 4-1) showed that, the wild-type Arabidopsis thaliana Col-0 (Columbia-0) was uncapable of growing roots on plates containing 2,4-D (0.3 μmol). Among the transgenic Arabidopsis thaliana, a few individuals of the Arabidopsis thaliana T1 seedlings transformed with pQY2312 (expressing QYD45, SEQ ID NO. 89); pQY2309 (expressing QYD42, SEQ ID NO. 83); pQY2310 (expressing QYD43, SEQ ID NO. 85); pQY2311 (expressing QYD44, SEQ ID NO. 87); pQY2313 (expressing QYD46, SEQ ID NO. 91); pQY2314 (expressing QYD47, SEQ ID NO. 93); pQY2315 (expressing QYD48, SEQ ID NO. 95); pQY2316 (expressing QYD49, SEQ ID NO. 97); pQY2318 (expressing QYD51, SEQ ID NO. 101); pQY2319 (expressing QYD52, SEQ ID NO. 103); pQY2320 (expressing QYD53, SEQ ID NO. 105); pQY2322 (expressing QYD55, SEQ ID NO. 109); pQY2323 (expressing QYD56, SEQ ID NO. 111); pQY2324 (expressing QYD57, SEQ ID NO. 113) grew seminal roots, showing tolerance to 2,4-D. In addition, the transgenic T1 seedlings were screened for drug-resistance on plates containing 2,4-DB (0.7 μmol) and MCPA (0.1 μmol) (FIGS. 4-2 and 4-3), respectively. The results were similar to those of 2,4-D screening. Among them, the T1 seedlings transformed with pQY2312 (expressing QYD45, SEQ ID NO. 89) had lower resistance to 2,4-D, but obvious resistance to MCPA, and some plants showed apparent root growth.

The transgenic Arabidopsis thaliana plants with normal root growth were transplanted into pots filled with soil for further cultivation and the T2 seeds were harvested for further identification.

4. Drug Resistance Testing of the T2 Generation Transgenic Plants

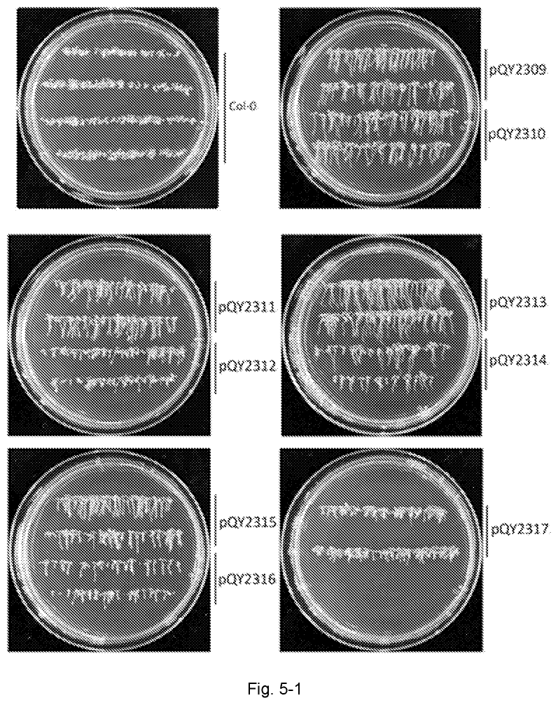

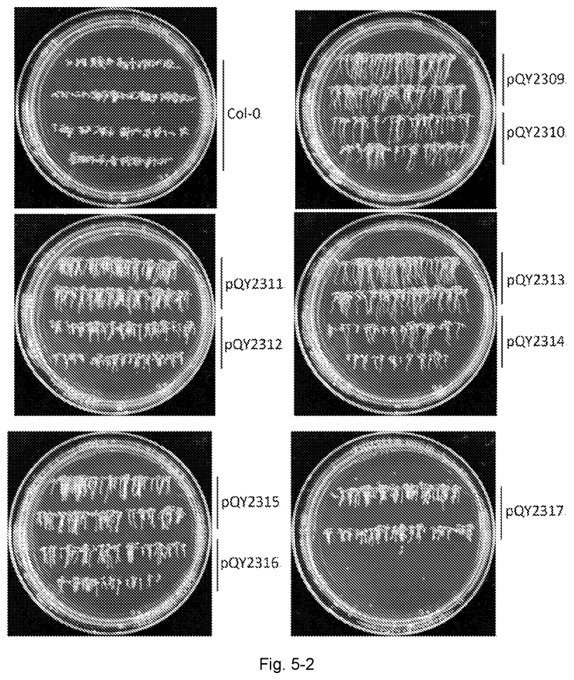

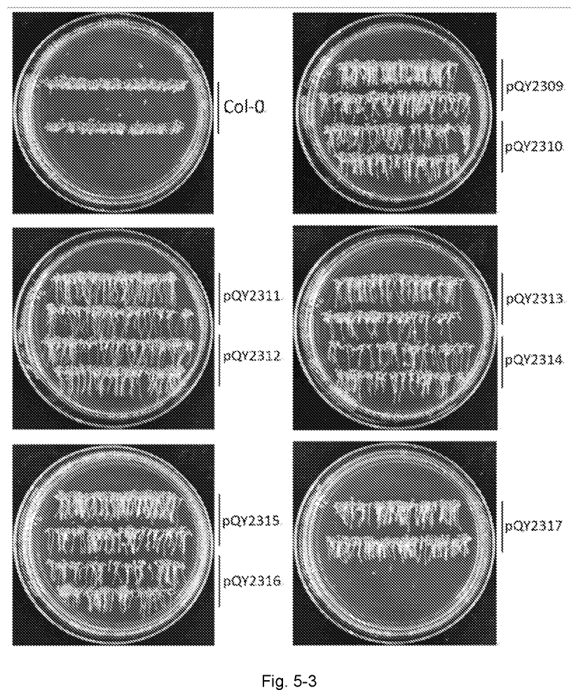

The transgenic lines with the nine vectors, pQY2309, pQY2310, pQY2311, pQY2312, pQY2313, pQY2314, pQY2315, pQY2316 and pQY2317, overexpressing AAD gene in the T2 generation were further tested on plate medium for their resistance to 2,4-D, 2,4-DB sodium and MCPA herbicide. Two events were tested for each transformant. Due to the resistance screening of T1 generation, most plants of the T2 generation showed resistance, in particular, pQY2312 showed obvious resistance to 2,4-DB sodium and MCPA, as shown in FIG. 5-1, FIG. 5-2 and FIG. 5-3.

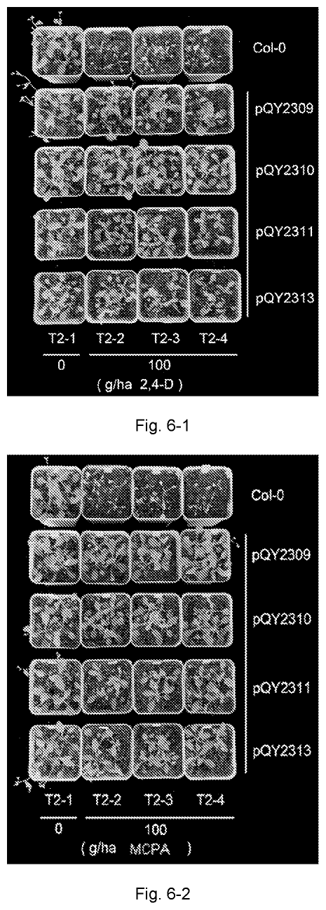

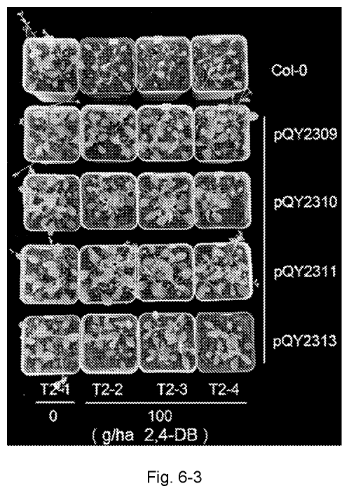

The T2 generation seedlings of four representative transformants, pQY2309, pQY2310, pQY2311 and pQY2313, were selected for further spray testing. Four lines were randomly selected from the T2 generation generated by transformation of each vector, and respectively foliage-sprayed with 2,4-D (100 g/ha), MCPA (100 g/ha) and 2,4-DB sodium (100 g/ha) when Arabidopsis thaliana seeds germinated, transplanted and the seedlings grew to 5-7 leaf. Non-transgenic wild-type Col-0 served as a negative control. The level of resistance was assessed 10 days after spraying. The results showed that all T2 seedlings of transgenic Arabidopsis thaliana had no apparent phytotoxicity, and all or almost all non-transgenic wild-type Arabidopsis thaliana died, as shown in FIG. 6-1, FIG. 6-2 and FIG. 6-3.

### Example 5

Construction and Genetic Transformation of a Maize Overexpression Vector

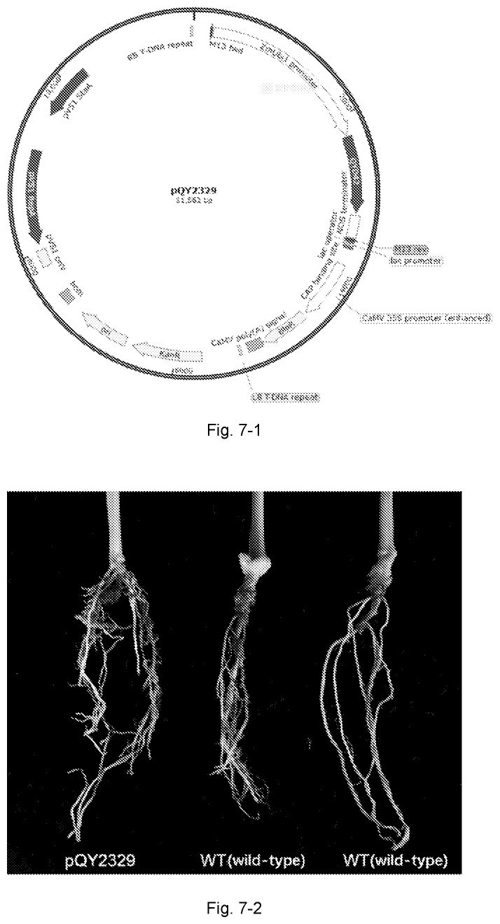

1. Vector construction: the overexpression vector was altered from pCambia3300, containing a Bar resistance gene that can be used as a selection marker for bialaphos (glufosinate). The AAD-like gene, QYD42, SEQ ID NO. 83, was linked to the pCambia3300 at the BamH I restriction site by seamless cloning to construct the transgenic vector pQY2329, of which the expression cassette consisted of a maize ubiquitin-1 promoter, the AAD gene and a Nos terminator, as shown in FIG. 7-1.

2. Genetic transformation of maizes: Maize transformation was carried out by the Agrobacterium-mediated immature embryo transformation method (Jones T, Lowe K, Hoerster G, et al. Maize Transformation Using the Morphogenic Genes Baby Boom and Wuschel2. Methods Mol Biol. 2019; 1864:81-93. Doi: 10.1007/978-1-4939-8778-8 6). The Agrobacterium (EHA105) comprising the pQY2329 overexpression vector was inoculated into YEP liquid medium (containing 25 mg/L Rifampicin and 50 mg/L Kanamycin), and shaking-cultured a t 28° C., 220 rmp/min overnight until the OD600 was about 1.0-1.5, then centrifuged at 6000 rmp/min for 10 min to collect the cells into a 50 ml centrifuge tube (sterilized). After discarding the supernatant, the cells were resuspended using MS infect culture medium (containing 68.5 g/L sucrose, 36 g/L glucose, pH 5.8) to OD550=0.8. Young panicles of the maize were taken 9-12 days after pollination to remove the bracts, inserted with a long handle gun forcep at the bottom, sterilized in the disinfectant of 1.6% sodium hypochlorite and 0.1% Tween 20 for 20 min, and then washed 3 times with sterile water, 5 min each time. The top 2-3 mm of the maize kernel was cut off using a scalpel and the immature embryo was picked and put into a 50 ml centrifuge tube. The Agrobacterium suspension was added and let stand for 5 min. The Agrobacterium solution was discarded and the immature embryo was transferred to MS co-culture medium (containing 20 g/L sucrose, 10 g/L glucose, 100 μM AS, 10 mg/L VC, 50 mg/L thymidine, 8 g/L agar, 0.5 g/L IVIES buffer, pH 5.8), and cultured in dark overnight at 22° C. The immature embryo was transferred to recovery medium after co-culture (containing MS salts, a large amount of salt of 0.6×N6, 1.68 g/L KNO₃, 0.6×B5 trace salt, 0.4×Eriksson's vitamins, 1×Murashige & Skoog vitamins, 0.2 mg/L VB1, 0.3 g/L casein hydrolysate, 20 g/L sucrose, 2 g/L proline, 0.6 g/L glucose, 100 mg/L cephalosporin, 150 mg/L Timentin, 8 g/L agar, pH 5.8) and cultured for one week, and then subcultured with fresh recovery media (containing 2 mg/L bialaphos) for every two weeks. After 4-6 weeks, the rapidly growing embryogenic callus was transferred to differentiation medium (containing 60 g/L sucrose, 0.5 mg/L zeatin, 0.1 mg/L thidiazuron, 1 mg/L 6-BA, 100 mg/L carbenicillin, 8 g/L agar, 2 mg/L bialaphos, pH 5.8) and cultured in dark for 2-3 weeks to induce buds formation. When the buds were 1-2 cm long, the callus was transferred to MS rooting medium (containing 40 g/L sucrose, 2 mg/L bialaphos, pH 5.8) and cultured under low level light (10-30 μmol/m²/s) for germinating and rooting, and finally transplanted to the greenhouse.

### Example 6

Herbicide-Resistance Capacity Testing of Transgenic Maizes

The T1 seedlings of the transgenic maize line obtained with the overexpression vector pQY2329 were planted, and first smeared with glufosinate (200 mg/L) on the leaves to confirm as transgenic, and when the seedling heights were about 20 cm, then foliage-sprayed with 2,4-D (4.48 kg/ha), along with the non-transgenic parent plant B104. The non-transgenic recipient parent plant served as a negative control. The level of resistance was assessed 15 days after spraying and the results showed that, the non-transgenic recipient parent had apparent phytotoxicity, root nodules, reduced root system and easily-lodging plant, while the root system of the transgenic maize grew normally without any growth inhibition, as shown in FIG. 7-2.

### Example 7

Construction and genetic transformation of a soybean overexpression vector

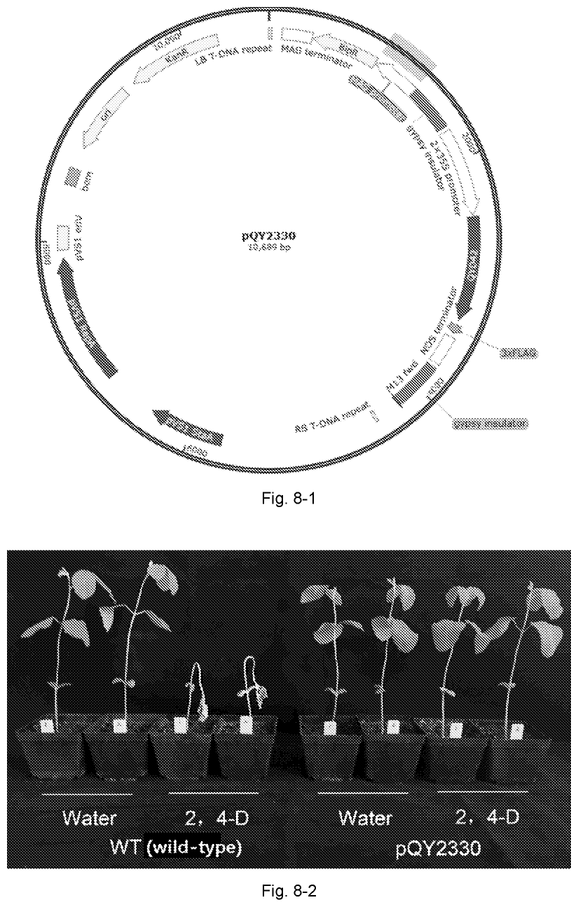

1. Vector construction: the pQY2330 vector (see FIG. 8-1) was used as the overexpression vector, containing a Bar resistance gene that can be used as a selection marker for glufosinate. The AAD-like gene, QYD42, SEQ ID NO. 83, was linked to the Xho I restriction site by seamless cloning to construct the transgenic expression vector pQY2330, of which the expression cassette consisted of a double CaMV35S promoter, the AAD-like gene QYD42 and a CaMV35S Nos terminator.

2. Genetic transformation of soybeans: Soybean transformation was carried out using the Agrobacterium-mediated cotyledon nodes transformation method (Pareddy D, Chennareddy S, Anthony G, et al. Improved soybean transformation for efficient and high throughput transgenic production[J]. Transgenic Research, 2020, 29(968)). A monoclone of the Agrobacterium (EHA105) comprising the overexpression vector pQY2330 was inoculated into 5 ml YEP liquid medium (containing 25 mg/L Rifampicin and 50 mg/L Kanamycin) and shaking-cultured at 220 rmp/min, 28° C. for 8 hours. And then 0.2-0.3 ml bacteria solution was transferred to 200 ml YEP liquid medium (containing 25 mg/L Rifampicin and 50 mg/L Kanamycin) and shaking-cultured at 220 rmp/min, 28° C. overnight until the bacteria reached the exponential growth stage, then centrifuged at 4000 rmp/min for 20 min to collect the cells into a 50 ml centrifuge tube (sterilized). After discarding the supernatant, the cell precipitates were resuspended using infect culture medium (B5 salts, B5 vitamins, 30 g/L sucrose, 1.67 mg/L BAP, 0.25 ml/L gibberellin, 3.9 g/L MES and 200 μM acetosyringone, pH 5.0) to OD600=0.6. Soybean seeds were disinfected with chlorine gas and stored in an uncovered container in a laminar flow unit to dissipate excess chlorine gas. The disinfected seeds were soaked in sterile water in petri dishes at 24° C. for 16 hours in the dark. The soybean seed was cut in half longitudinally along the seed hilum using a No. 10 scalpel, each half containing half of the embryo and cotyledon. The distal part of the embryonic axis was excised, and approximately half to one third of the embryonic axis was still attached to the end of the cotyledon node. The seed explant with partial embryonic axis was immersed in an infection liquid for 30 minutes, then transferred to co-culture medium containing B5 salts and vitamins and covered with filter paper. The explant was cultured for 5 days at 24° C. under a photoperiod of 18-hour light and 6-hour dark, with a light intensity of 80-90 μmol/m²/s. Five days later, the explant was rinsed with liquid induction culture medium (containing B5 salts, B5 vitamins, 1.11 mg/L BAP, 30 g/L sucrose, 28 mg/L ferrous sulfate, 38 mg/L Na₂EDTA, 0.6 g/L IVIES, 50 mg/L Kanamycin, 200 mg/L cefotaxime and 100 mg/L Timentin, pH 5.7). The explant was transferred to induction culture medium (containing 7 g/L agar), with the curved surface of cotyledon facing downward and the end of cotyledon node embedded in the medium. After 2 weeks of cultivation, the explant was transferred to screening medium (containing 6 mg/L glufosinate) for another 2 weeks. The cotyledons were excised from the base portion of the explant, and the horizontal “bud pad” containing the embryonic axis was transferred to subculture medium (containing MS salts, 30 g/L sucrose, 28 mg/L ferrous sulfate, 0.6 g/L IVIES, 38 mg/L Na₂EDTA, 0.1 mg/L IAA, 1 mg/L zeatin riboside, 0.5 mg/L GA3, 50 mg/L asparagine, 100 mg/L pyroglutamic acid, 50 mg/L Kanamycin, 200 mg/L cefotaxime, 50 mg/L Timentin, 6 mg/L glufosinate, 7 g/L agar, pH 5.7) and subcultured every 2 weeks. The bud base was excised off and soaked in 1 mg/L indole-3-butyric acid for 1-3 minutes to promote rooting. Then, the bud base was transferred to rooting medium (containing MS salts, B5 vitamins, 20 g/L sucrose, 28 mg/L ferrous sulfate, 0.59 g/L IVIES, 38 mg/L Na₂EDTA, 100 mg/L pyroglutamic acid, 50 mg/L asparagine, 7 g/L agar, pH 5.6) for 1-2 weeks, and then the rooted shoot was transplanted into soil to acclimate the seedling, and finally transplanted to the greenhouse. During the cultivation of TO generation seedlings, glufosinate was smeared (150 mg/L) on the leaves to further identify whether they were transgenic.

### Example 8

After obtaining the T1 generation seeds and confirming the transgene through genotype identification and glufosinate smear identification, the T1 seedlings were spray-tested. 2,4-D (4.48 kg/ha) were foliage-sprayed on soybean seedlings when the seedlings were about 20 cm high and the non-transgenic recipient parents served as negative controls. The level of resistance was assessed 10 days after spraying. The results showed that all non-transgenic recipient parents died, while the transgenic soybeans (pQY2330) had no phytotoxicity, and no growth inhibition was observed, as shown in FIG. 8-2.

All publications and patent applications mentioned in the specification are hereby incorporated herein by reference, just as each publication or patent application is separately or particularly incorporated by reference into herein.

Although the foregoing invention has been described in detail through illustrations and examples for clear understanding, obviously some changes and modifications can be implemented within the scope of claims attached. Such changes and modifications are within the scope of the present invention.

1. A recombinant DNA molecule comprising a nucleic acid sequence selected from:
(1) a nucleic acid sequence encoding a protein or a bioactive fragment thereof that comprises an amino acid sequence having at least 90%, at least 95%, at least 96%, at least 97%, at least 98%, at least 99% or 100% sequence identity with at least one amino acid sequence selected from the following group: SEQ ID NO: 41, SEQ ID NO: 51, SEQ ID NO: 59, SEQ ID NO: 63, SEQ ID NO: 65, SEQ ID NO: 67, SEQ ID NO: 69, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 123, SEQ ID NO: 143, SEQ ID NO: 145, SEQ ID NO: 147, SEQ ID NO: 149 and SEQ ID NO: 151, or a complementary sequence thereof;
(2) a nucleic acid sequence as set forth in: SEQ ID NO: 42, SEQ ID NO: 52, SEQ ID NO: 60, SEQ ID NO: 64, SEQ ID NO: 66, SEQ ID NO: 68, SEQ ID NO: 70, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 124, SEQ ID NO:144, SEQ ID NO: 146, SEQ ID NO: 148, SEQ ID NO: 150 or SEQ ID NO: 152, or a complementary sequence thereof.
(3) a nucleic acid sequence that hybridizes to the sequence as shown in (1) or (2) under stringent conditions; or
(4) a nucleic acid sequence encoding the same amino acid sequence as the sequence shown in (1) or (2) due to degeneracy of genetic code, or a complementary sequence thereof.

2. The recombinant DNA molecule according to claim 1, wherein it is characterized in that the recombinant DNA molecule is operably linked to a heterologous promoter functional in a plant cell.

3. A DNA construct comprising a heterologous promoter functional in a plant cell that is operably linked to the recombinant DNA molecule according to claim 1 or 2.

4. The DNA construct according to claim 3, wherein it exists in the genome of a transgenic plant.

5. A protein or a bioactive fragment thereof encoded by the recombinant DNA molecule according to claim 1 or 2.

6. A protein or a bioactive fragment thereof, wherein the amino acid sequence thereof has at least 90%, at least 95%, at least 96%, at least 97%, at least 98%, at least 99% or 100% sequence identity with at least one amino acid sequence selected from the following group: SEQ ID NO: 41, SEQ ID NO: 51, SEQ ID NO: 59, SEQ ID NO: 63, SEQ ID NO: 65, SEQ ID NO: 67, SEQ ID NO: 69, SEQ ID NO: 83, SEQ ID NO: 85, SEQ ID NO: 87, SEQ ID NO:89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 123, SEQ ID NO: 143, SEQ ID NO: 145, SEQ ID NO: 147, SEQ ID NO: 149 and SEQ ID NO: 151.

7. The protein or a bioactive fragment thereof according to claim 5 or 6, wherein it has oxygenase activity on at least one of the following types of herbicides: hormone herbicides, ACCase inhibitor herbicides.

8. A plant, seed, cell or plant part comprising the recombinant DNA molecule according to claim 1 or 2, the DNA construct according to claim 3 or 4, or the protein or a bioactive fragment thereof according to any one of claims 5 to 7.

9. The plant, seed, cell or plant part according to claim 8, wherein it has tolerance to at least one of the following types of herbicides: hormone herbicides, ACCase inhibitor herbicides.

10. An isolated polynucleotide comprising the recombinant DNA molecule according to claim 1 or 2, the DNA construct according to claim 3 or 4, or a nucleic acid sequence encoding the protein or a bioactive fragment thereof according to any one of claims 5 to 7, or a complementary sequence thereof.

11. A plant genome comprising the polynucleotide according to claim 10.

12. A vector comprising the polynucleotide according to 10 and a homologous promoter operably linked thereto.

13. The vector according to claim 12, wherein it is characterized in that the promoter is an inducible promoter or a promoter of the gene itself in a plant genome.

14. A host cell comprising the polynucleotide according to claim 10 or the vector according to claim 12 or 13.

15. A method for producing or improving a plant or seed with herbicide tolerance, comprising transforming a plant cell or tissue with the recombinant DNA molecule according to 1 or 2 or the DNA construct according to 3 or 4, and regenerating a herbicide-tolerant plant from the transformed plant cell or tissue.

16. A plant seed produced by the method according to claim 15.

17. A method for conferring herbicide tolerance on a plant, seed, cell or plant part, comprising expressing the protein or a bioactive fragment thereof according to any one of claims 5 to 7, in the plant, seed, cell or plant part;
or, comprising crossing a plant that expresses the protein or a bioactive fragment thereof according to any one of claims 5 to 7 with another plant, and screening for a plant, seed, cell or plant part capable of producing or improving herbicide tolerance;
or, comprising gene editing the plant, seed, cell or plant part so as to express the protein or a bioactive fragment according to any one of claims 5 to 7 therein.

18. The method according to claim 17, wherein it is characterized in that the plant, seed, cell or plant part comprises the DNA construct according to claim 3 or 4.

19. Use of the recombinant DNA molecule according to claim 1 or 2, the DNA construct according to claim 3 or 4, the protein or a bioactive fragment thereof according to any one of claims 5 to 7, the polynucleotide according to claim 10, the plant genome according to claim 11, the vector according to claim 12 or 13, or the host cell according to claim 14 for producing or improving herbicide tolerance of a plant, seed, cell or plant part.

20. A method for controlling weeds in a plant growing area, comprising exposing a plant growing area, where a herbicide-tolerant plant or seed grows, to the herbicide, wherein the plant or seed comprises the plant or seed according to claim 16, a plant or seed prepared by the method according to claim 15, 17 or 18, or a plant or seed comprising the recombinant DNA molecule according to claim 1 or 2.

21. The method according to claim 15, 17, 18 or 20 or the use according to claim 19, wherein it is characterized in that the herbicide is selected from at least one of the following types: hormone herbicides, ACCase inhibitor herbicides.

22. The protein or a bioactive fragment thereof according to claim 7, the plant, seed, cell or plant part according to claim 9, the method according to claim 15, 17, 18 or 20, or the use according to claim 19, wherein it is characterized in that the herbicide is selected from at least one of the following types: pyridinecarboxylic acids, benzoic acids, phenoxycarboxylic acids and aryloxy phenoxy propionates; preferably, the herbicide is selected from at least one of the following chemical compounds: fluroxypyr, triclopyr, dicamba, 2,4-D, 2,4-DB, 2,4-DB sodium, 2,4-D isooctyl ester, MCPA and quizalofop-P-ethyl.
